(19) United States
(12) Patent Application Publication
(10) Pub. No.: US 2024/0300882 A1
(43) Pub. Date: Sep. 12, 2024

(54) CRYSTALLINE FORMS OF A DIFFUSION ENHANCING COMPOUND

(71) Applicant: DIFFUSION PHARMACEUTICALS LLC, Charlottesville, VA (US)

(72) Inventors: Beth HAAS, Bishop, GA (US); David JONAITIS, Brookston, IN (US); Cassie LINDENBERGER, Lafayette, IN (US); Dirk ELEND, Aarau (CH)

(21) Appl. No.: 18/654,846

(22) Filed: May 3, 2024

Related U.S. Application Data
(63) Continuation of application No. PCT/US2022/079247, filed on Nov. 3, 2022.
(60) Provisional application No. 63/275,444, filed on Nov. 3, 2021.

(51) Int. Cl. C07C 57/13; A61K 31/202

(52) U.S. Cl. CPC C07C 57/13; A61K 31/202; C07B 2200/13

(57) ABSTRACT

Provided are crystals comprising trans sodium crocetinate, compositions comprising the same, and methods of making and using such crystals.

This application claims priority to U.S. Provisional Application No. 63/275,444 filed Nov. 3, 2021, which is hereby incorporated by reference in its entirety.

### FIELD

Provided are crystals comprising trans sodium crocetinate, compositions comprising the same, and methods of making and using such crystals.

### BACKGROUND

Trans sodium crocetinate (TSC) is discussed in U.S. Pat. Nos. 7,351,844, 7,759,506, 8,030,350, 8,293,804, 8,974,822, and 10,130,689, U.S. Patent Publication Nos. 2019/0083439 and 2021/0244698, and International Publication No. WO 2021/202735. The compound is effective in improving oxygen diffusivity between red blood cells and body tissues and may be used to treat a variety of conditions.

Active pharmaceutical ingredients can exist in different physical forms (e.g., liquid or solid in different crystalline, amorphous, hydrate, or solvate forms), which can vary the processability, stability, solubility, bioavailability, or pharmacokinetics (absorption, distribution, metabolism, excretion, or the like) and/or bioequivalency of the active pharmaceutical ingredient and pharmaceutical compositions comprising it.

In addition, isolation of a crystalline solid form may have advantages over an amorphous solid in terms of purification, stability, and solid handling. Discovery of a crystalline solid may provide a manufacturing route that allows for more control over a synthetic step than if an amorphous solid is produced.

Thus, there is a need to identify active pharmaceutical ingredients having an advantageous physical form (e.g., in solid, liquid, crystalline, hydrate, solvate, or amorphous forms).

### SUMMARY

Trans sodium crocetinate (TSC) is shown as Formula I below

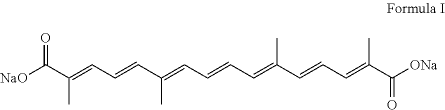

It has been found that trans sodium crocetinate (Formula I) exists as multiple polymorphic forms (Crystalline Forms A-G and J are described herein). The forms differ from each other with respect to their physical properties, spectral data, stability, and methods of preparation. The forms can be used as active pharmaceutical ingredients or as intermediates to a final formulation of trans sodium crocetinate that are useful to remove impurities and ensure consistency and control in the formulation process. The forms may be isolated in high purity and may exhibit stability even under stressed conditions. Competitive slurry experiments involving Crystalline Forms A-F indicate that Crystalline Form A is the most stable form under non-aqueous conditions and Crystalline Form B is the most stable form under aqueous conditions. Specifically, Crystalline Form A is more stable than Forms B through E between 2 and 50° C. in methanol. Crystalline Form A is more stable than Forms B through F between 2 and 55° C. in absolute ethanol and absolute ethanol containing either 10% methanol or toluene. Crystalline Form B is more stable than Forms A, C, D, E, and F between 2 and 55° C. in 1:1 ethanol:water (v/v).

Crystalline Form A remains unchanged under various heating and humidity conditions. For instance, Crystalline Form A remains unchanged after DVS analysis where the sample is exposed up to 95% relative humidity (RH) and down to 5% RH, remains unchanged when heated to 100° C. for one minute, and remains unchanged for up to 4 weeks at 25° C./60% relative humidity (RH) and 40° C./75% RH. Upon heating, Crystalline Form B converts to Crystalline Form F.

Crystalline Form F is easy to filter. In addition, in the absence of Crystalline Form A, Crystalline Form F will remain Crystalline Form F (see examples). Crystalline Form F also remains unchanged under various heating and humidity conditions. For instance, Crystalline Form F remains unchanged after DVS analysis where the sample is exposed up to 95% relative humidity (RH) and down to 5% RH and remains unchanged for up to 4 weeks at 25° C./60% relative humidity (RH) and 40° C./75% RH.

Form H, upon exposure to different relative humidities, changes form (see Example 26).

Provided herein is trans sodium crocetinate (Formula I) in crystalline form (as described below), which may be used in the pharmaceutical compositions, methods, and kits described in any of U.S. Pat. Nos. 7,351,844, 7,759,506, 8,030,350, 8,293,804, 8,974,822, and 10,130,689, U.S. Patent Publication Nos. 2019/0083439 and 2021/0244698, and International Publication Nos. WO 2021/202735 and WO 2022/150716, each of which is hereby incorporated by reference in its entirety. For instance, provided is trans sodium crocetinate (Formula I) in hydrate (e.g., hemi-hydrate or monohydrate) crystalline form. For instance, also provided is trans sodium crocetinate (Formula I) in non-solvate non-hydrate crystalline form.

Accordingly, provided is Crystalline Form A (also called Form A herein) of trans sodium crocetinate (Formula I above). Further provided is Crystalline Form A as follows:
A.1 Crystalline Form A, wherein the Crystalline Form A exhibits an XRPD pattern comprising 2-theta (°) values 4.2, 8.4, 15.8, and 17.3, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. Or, for instance, Crystalline Form A, wherein the Crystalline Form A exhibits an XRPD pattern comprising d-spacing (Å) values of 20.9, 10.5, 5.6, and 5.1.
A.2 Crystalline Form A or A.1, wherein the Crystalline Form A exhibits an XRPD pattern comprising 2-theta (°) values of 4.2, 8.4, 15.8, 17.3, and 19.5, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. Or, for instance, Crystalline Form A or A.1, wherein the Crystalline Form A exhibits an XRPD pattern comprising d-spacing (Å) values of 20.9, 10.5, 5.6, 5.1, and 4.6.
A.3 Any of Crystalline Form A, A.1, or A.2, wherein the Crystalline Form A exhibits an XRPD pattern comprising at least three, e.g., at least four, e.g., at least five, e.g., at least ten, e.g., at least fifteen, e.g., at least twenty, e.g., at least twenty-five, e.g., at least thirty, 2-theta (°) values selected from the group consisting of 4.2, 5.2, 6.1, 8.4, 10.4, 11.7, 15.8, 16.8, 17.3, 18.4, 19.5, 20.4, 21.0, 22.6, 22.9, 23.6, 23.9, 24.4, 25.2, 25.9, 26.9, 27.4, 28.6, 29.3, 29.6, 29.9, 30.9, 31.2, 31.9, 32.2, 33.5, 33.9, 35.1, and 38.3, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. For instance, any of Crystalline Form A,
A.1, or A.2, wherein the Crystalline Form A exhibits an XRPD pattern comprising at least four, e.g., at least five, e.g., at least ten, e.g., at least fifteen, e.g., at least twenty, 2-theta (°) values selected from the group consisting of 4.2, 8.4, 15.8, 16.8, 17.3, 18.4, 19.5, 21.0, 22.6, 23.9, 25.2, 25.9, 26.9, 28.6, 29.3, 29.9, 30.9, 33.5, 33.9, 35.1, and 38.3, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
A.4 Any of Crystalline Form A or A.1-A.3, wherein the Crystalline Form A exhibits an XRPD pattern comprising 2-theta (°) values of 4.2, 8.4, 15.8, 16.8, 17.3, 18.4, 19.5, 21.0, 22.6, 23.9, 25.2, 25.9, 26.9, 28.6, 29.3, 29.9, 30.9, 33.5, 33.9, 35.1, and 38.3, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. For instance, any of Crystalline Form A or A.1-A.3, wherein the Crystalline Form A exhibits an XRPD pattern comprising 2-theta (°) values of 4.2, 5.2, 6.1, 8.4, 10.4, 11.7, 15.8, 16.8, 17.3, 18.4, 19.5, 20.4, 21.0, 22.6, 22.9, 23.6, 23.9, 24.4, 25.2, 25.9, 26.9, 27.4, 28.6, 29.3, 29.6, 29.9, 30.9, 31.2, 31.9, 32.2, 33.5, 33.9, 35.1, and 38.3, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
A.5 Any of Crystalline Form A or A.1-A.4, wherein the Crystalline Form A exhibits an XRPD pattern comprising at least three, e.g., at least five, e.g., at least ten, e.g., at least fifteen, e.g., at least twenty, e.g., at least twenty-five, e.g., at least thirty, 2-theta (°) values selected from the group consisting of 4.22, 5.17, 6.06, 8.38, 10.35, 11.68, 15.80, 16.75, 17.34, 18.43, 19.47, 20.40, 20.97, 22.62, 22.94, 23.60, 23.93, 24.35, 25.24, 25.91, 26.91, 27.35, 28.57, 29.30, 29.56, 29.92, 30.89, 31.15, 31.92, 32.16, 33.47, 33.90, 35.06, and 38.29, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
A.6 Any of Crystalline Form A or A.1-A.5, wherein the Crystalline Form A exhibits an XRPD pattern comprising 2-theta (°) values of 4.22, 5.17, 6.06, 8.38, 10.35, 11.68, 15.80, 16.75, 17.34, 18.43, 19.47, 20.40, 20.97, 22.62, 22.94, 23.60, 23.93, 24.35, 25.24, 25.91, 26.91, 27.35, 28.57, 29.30, 29.56, 29.92, 30.89, 31.15, 31.92, 32.16, 33.47, 33.90, 35.06, and 38.29, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
A.7 Any of Crystalline Form A or A.1-A.6, wherein the Crystalline Form A exhibits an XRPD pattern comprising at least three, e.g., at least five, e.g., at least ten, e.g., at least fifteen, e.g., at least twenty, e.g., at least twenty-five, d-spacing (Å) values selected from the group consisting of 20.9, 17.1, 14.6, 10.5, 8.5, 7.6, 5.6, 5.3, 5.1, 4.8, 4.6, 4.3, 4.2, 3.9, 3.8, 3.7, 3.5, 3.4, 3.3, 3.1, 3.0, 2.9, 2.8, 2.7, 2.6, and 2.3. For instance, any of Crystalline Form A or A.1-A.6, wherein the Crystalline Form A exhibits an XRPD pattern comprising at least four, e.g., at least five, e.g., at least ten, e.g., at least fifteen, d-spacing (Å) values selected from the group consisting of 20.9, 10.5, 5.6, 5.3, 5.1, 4.8, 4.6, 4.2, 3.9, 3.7, 3.5, 3.4, 3.3, 3.1, 3.0, 2.9, 2.7, 2.6, and 2.3.
A.8 Any of Crystalline Form A or A.1-A.7, wherein the Crystalline Form A exhibits an XRPD pattern comprising d-spacing (Å) values of 20.9, 10.5, 5.6, 5.3, 5.1, 4.8, 4.6, 4.2, 3.9, 3.7, 3.5, 3.4, 3.3, 3.1, 3.0, 2.9, 2.7, 2.6, and 2.3. For instance, any of Crystalline Form A or A.1-A.7, wherein the Crystalline Form A exhibits an XRPD pattern comprising d-spacing (Å) values of 20.9, 17.1, 14.6, 10.5, 8.5, 7.6, 5.6, 5.3, 5.1, 4.8, 4.6, 4.3, 4.2, 3.9, 3.8, 3.7, 3.5, 3.4, 3.3, 3.1, 3.0, 2.9, 2.8, 2.7, 2.6, and 2.3.
A.9 Any of Crystalline Form A or A.1-A.8, wherein the Crystalline Form A exhibits an XRPD pattern comprising at least three, e.g., at least five, e.g., at least ten, e.g., at least fifteen, e.g., at least twenty, e.g., at least twenty-five, e.g., at least thirty, d-spacing (Å) values selected from the group consisting of 20.94, 17.07, 14.58, 10.55, 8.54, 7.57, 5.61, 5.29, 5.11, 4.81, 4.56, 4.35, 4.23, 3.93, 3.87, 3.77, 3.72, 3.65, 3.53, 3.44, 3.31, 3.26, 3.12, 3.05, 3.02, 2.98, 2.89, 2.87, 2.80, 2.78, 2.68, 2.64, 2.56, and 2.35. For instance, any of Crystalline Form A or A.1-A.8, wherein the Crystalline Form A exhibits an XRPD pattern comprising at least four, e.g., at least five, e.g., at least ten, e.g., at least fifteen, e.g., at least twenty, d-spacing (Å) values selected from the group consisting of 20.94, 10.55, 5.61, 5.29, 5.11, 4.81, 4.56, 4.23, 3.93, 3.72, 3.53, 3.44, 3.31, 3.12, 3.05, 2.98, 2.89, 2.68, 2.64, 2.56, and 2.35.
A.10 Any of Crystalline Form A or A.1-A.9, wherein the Crystalline Form A exhibits an XRPD pattern comprising d-spacing (Å) values of 20.94, 10.55, 5.61, 5.29, 5.11, 4.81, 4.56, 4.23, 3.93, 3.72, 3.53, 3.44, 3.31, 3.12, 3.05, 2.98, 2.89, 2.68, 2.64, 2.56, and 2.35. For instance, any of Crystalline Form A or A.1-A.9, wherein the Crystalline Form A exhibits an XRPD pattern comprising d-spacing (Å) values of 20.94, 17.07, 14.58, 10.55, 8.54, 7.57, 5.61, 5.29, 5.11, 4.81, 4.56, 4.35, 4.23, 3.93, 3.87, 3.77, 3.72, 3.65, 3.53, 3.44, 3.31, 3.26, 3.12, 3.05, 3.02, 2.98, 2.89, 2.87, 2.80, 2.78, 2.68, 2.64, 2.56, and 2.35.
A.11 Any of Crystalline Form A or A.1-A.10, wherein the Crystalline Form A exhibits an XRPD pattern comprising at least three, e.g., at least five, e.g., at least ten, e.g., at least fifteen, e.g., at least twenty, e.g., at least twenty-five, e.g., at least thirty, d-spacing (Å) values selected from the group consisting of 20.943, 17.065, 14.575, 10.548, 8.540, 7.573, 5.606, 5.289, 5.110, 4.811, 4.557, 4.349, 4.234, 3.928, 3.874, 3.766, 3.715, 3.652, 3.526, 3.436, 3.311, 3.258, 3.122, 3.045, 3.020, 2.984, 2.892, 2.869, 2.801, 2.781, 2.675, 2.642, 2.558, and 2.349. For instance, any of Crystalline Form A or A.1-A.10, wherein the Crystalline Form A exhibits an XRPD pattern comprising at least four, e.g., at least five, e.g., at least ten, e.g., at least fifteen, e.g., at least twenty, d-spacing (Å) values selected from the group consisting of 20.943, 10.548, 5.606, 5.289, 5.110, 4.811, 4.557, 4.234, 3.928, 3.715, 3.526, 3.436, 3.311, 3.122, 3.045, 2.984, 2.892, 2.675, 2.642, 2.558, and 2.349.
A.12 Any of Crystalline Form A or A.1-A.11, wherein the Crystalline Form A exhibits an XRPD pattern comprising d-spacing (Å) values of 20.943, 10.548, 5.606, 5.289, 5.110, 4.811, 4.557, 4.234, 3.928, 3.715, 3.526, 3.436, 3.311, 3.122, 3.045, 2.984, 2.892, 2.675, 2.642, 2.558, and 2.349. For instance, any of Crystalline Form A or A.1-A.11, wherein the Crystalline Form A exhibits an XRPD pattern comprising d-spacing (Å) values of 20.943, 17.065, 14.575, 10.548, 8.540, 7.573, 5.606, 5.289, 5.110, 4.811, 4.557, 4.349, 4.234, 3.928, 3.874, 3.766, 3.715, 3.652, 3.526, 3.436, 3.311, 3.258, 3.122, 3.045, 3.020, 2.984, 2.892, 2.869, 2.801, 2.781, 2.675, 2.642, 2.558, and 2.349.
A.13 Any of Crystalline Form A or A.1-A.12, wherein the Crystalline Form A exhibits an XRPD pattern comprising at least three, e.g., at least five, e.g., at least ten, e.g., at least fifteen, e.g., at least twenty, e.g., at least twenty-five, e.g., at least thirty, 2-theta (°) values selected from those set forth in Table A below:

TABLE A
Form A

| Peak Position | Relative | d-spacing |
|---|---|---|
| (°2θ)^{a} | Intensity | value (Å) |
| 4.22 | 53.99 | 20.9427 |
| 5.17 | 7.17 | 17.0650 |
| 6.06 | 1.57 | 14.5751 |
| 8.38 | 26.21 | 10.5480 |
| 10.35 | 4.49 | 8.5400 |
| 11.68 | 1.63 | 7.5732 |
| 15.80 | 12.53 | 5.6057 |
| 16.75 | 1.58 | 5.2894 |
| 17.34 | 63.17 | 5.1096 |
| 18.43 | 2.40 | 4.8112 |
| 19.47 | 10.03 | 4.5565 |
| 20.40 | 2.30 | 4.3494 |
| 20.97 | 1.51 | 4.2336 |
| 22.62 | 42.12 | 3.9281 |
| 22.94 | 20.33 | 3.8742 |
| 23.60 | 3.98 | 3.7661 |
| 23.93 | 3.77 | 3.7153 |
| 24.35 | 1.40 | 3.6520 |
| 25.24 | 2.88 | 3.5257 |
| 25.91 | 100.00 | 3.4358 |
| 26.91 | 8.00 | 3.3110 |
| 27.35 | 1.91 | 3.2578 |
| 28.57 | 1.89 | 3.1216 |
| 29.30 | 4.04 | 3.0454 |
| 29.56 | 1.35 | 3.0197 |
| 29.92 | 6.36 | 2.9836 |
| 30.89 | 15.02 | 2.8922 |
| 31.15 | 23.21 | 2.8688 |
| 31.92 | 2.58 | 2.8013 |
| 32.16 | 5.06 | 2.7812 |
| 33.47 | 2.67 | 2.6751 |
| 33.90 | 4.30 | 2.6421 |
| 35.06 | 3.39 | 2.5577 |
| 38.29 | 10.88 | 2.3489, |

^{a}+/−0.2°2θ per USP guidelines

e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
A.14 Any of Crystalline Form A or A.1-A.13, wherein the Crystalline Form A exhibits an XRPD pattern comprising the 2-theta (°) values set forth in Table A in Crystalline Form A.13, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
A.15 Any of Crystalline Form A or A.1-A.14, wherein the Crystalline Form A exhibits an XRPD pattern comprising at least three, e.g., at least five, e.g., at least ten, e.g., at least fifteen, e.g., at least twenty, e.g., at least twenty-five, e.g., at least thirty, d-spacing (Å) values selected from those set forth in Table A in Crystalline Form A.13.
A.16 Any of Crystalline Form A or A.1-A.15, wherein the Crystalline Form A exhibits an XRPD pattern comprising the d-spacing (Å) values set forth in Table A in Crystalline Form A.13.
A.17 Any of Crystalline Form A or A.1-A.16, wherein the Crystalline Form A exhibits an XRPD pattern comprising at least four, e.g., at least five, e.g., at least ten, e.g., at least fifteen, e.g., at least twenty, 2-theta (°) values selected from those set forth in Table AA below:

TABLE AA

| | Relative | d-spacing |
|---|---|---|
| °2θ | Intensity | value (Å) |
| 4.2 | 54 | 20.9427 |
| 8.4 | 26 | 10.5480 |
| 15.8 | 13 | 5.6057 |
| 16.8 | 2 | 5.2894 |
| 17.3 | 63 | 5.1096 |
| 18.4 | 2 | 4.8112 |
| 19.5 | 10 | 4.5565 |
| 21.0 | 2 | 4.2336 |
| 22.6 | 42 | 3.9281 |
| 23.9 | 4 | 3.7153 |
| 25.2 | 3 | 3.5257 |
| 25.9 | 100 | 3.4358 |
| 26.9 | 8 | 3.3110 |
| 28.6 | 2 | 3.1216 |
| 29.3 | 4 | 3.0454 |
| 29.9 | 6 | 2.9836 |
| 30.9 | 15 | 2.8922 |
| 33.5 | 3 | 2.6751 |
| 33.9 | 4 | 2.6421 |
| 35.1 | 3 | 2.5577 |
| 38.3 | 11 | 2.3489 |
| — | — | —, |

e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
A.18 Any of Crystalline Form A or A.1-A.17, wherein the Crystalline Form A exhibits an XRPD pattern comprising the 2-theta (°) values set forth in Table AA in Crystalline Form A.17, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
A.19 Any of Crystalline Form A or A.1-A.18, wherein the Crystalline Form A exhibits an XRPD pattern comprising at least four, e.g., at least five, e.g., at least ten, e.g., at least fifteen, e.g., at least twenty, d-spacing (Å) values selected from those set forth in Table AA in Crystalline Form A.17.
A.20 Any of Crystalline Form A or A.1-A.19, wherein the Crystalline Form A exhibits an XRPD pattern comprising the d-spacing (Å) values set forth in Table AA in Crystalline Form A.17.
A.21 Any of Crystalline Form A or A.1-A.20, wherein the Crystalline Form A exhibits an XRPD pattern comprising characteristic 2-theta (°) values selected from any of the 2-theta (°) values set forth in any of Crystalline Form A or A.1-A.20 above, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
A.22 Any of Crystalline Form A or A.1-A.21, wherein the Crystalline Form A exhibits an XRPD pattern comprising representative 2-theta (°) values selected from any of the 2-theta (°) values set forth in any of Crystalline Form A or A.1-A.20 above, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
A.23 Any of Crystalline Form A or A.1-A.22, wherein the Crystalline Form A exhibits an XRPD pattern comprising characteristic d-spacing (Å) values selected from any of the d-spacing (Å) values set forth in any of Crystalline Form A or A.1-A.20 above.
A.24 Any of Crystalline Form A or A.1-A.23, wherein the Crystalline Form A exhibits an XRPD pattern comprising representative d-spacing (Å) values selected from any of the d-spacing (Å) values set forth in any of Crystalline Form A or A.1-A.20 above.
A.25 Any of Crystalline Form A or A.1-A.24, wherein the Crystalline Form A exhibits an XRPD pattern comprising at least three, e.g., at least five, e.g., at least ten, e.g., at least fifteen, e.g., at least twenty, e.g., at least twenty-five, e.g., at least thirty, e.g., all, of the peaks of the XRPD shown in FIG. 1, FIG. 2, and/or FIG. 12, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
A.26 Any of Crystalline Form A or A.1-A.25, wherein the Crystalline Form A exhibits an XRPD pattern comprising the characteristic peaks of the XRPD shown in FIG. 1, FIG. 2, and/or FIG. 12, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
A.27 Any of Crystalline Form A or A.1-A.26, wherein the Crystalline Form A exhibits an XRPD pattern comprising the representative peaks of the XRPD shown in FIG. 1, FIG. 2, and/or FIG. 12, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
A.28 Any of Crystalline Form A or A.1-A.27, wherein the Crystalline Form A exhibits an XRPD corresponding to FIG. 1, FIG. 2, and/or FIG. 12, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
A.29 Any of Crystalline Form A or A.1-A.28, wherein the Crystalline Form A exhibits a differential scanning calorimetry (DSC) thermogram comprising an endotherm between 100-110° C., e.g., between 101-106° C., e.g., between 102-106° C. For instance, any of Crystalline Form A or A.1-A.28, wherein the Crystalline Form A exhibits a differential scanning calorimetry (DSC) thermogram comprising an endotherm at 102° C. (e.g., 101.9° C.). Or, for instance, any of Crystalline Form A or A.1-A.28, wherein the Crystalline Form A exhibits a differential scanning calorimetry (DSC) thermogram comprising an endotherm at 106° C. (e.g., 105.7° C.).
A.30 Any of Crystalline Form A or A.1-A.29, wherein the Crystalline Form A exhibits a differential scanning calorimetry (DSC) thermogram comprising an endotherm between 195-205° C., e.g., between 195-200° C. e.g., 200° C. (e.g., 199.6° C.).
A.31 Any of Crystalline Form A or A.1-A.30, wherein the Crystalline Form A exhibits a differential scanning calorimetry (DSC) thermogram as shown in FIG. 3 and/or FIG. 5.
A.32 Any of Crystalline Form A or A.1-A.31, wherein the Crystalline Form A exhibits a thermogravimetric analysis (TGA) thermogram comprising weight loss (e.g., a loss of water) between start (e.g., 25° C.) and 80° C., e.g., between start (e.g., 25° C.) and 75° C., e.g., a weight loss of 2-3 weight %, e.g., a weight loss of 2.4-2.5 weight %.
A.33 Any of Crystalline Form A or A.1-A.32, wherein the Crystalline Form A exhibits a thermogravimetric analysis (TGA) thermogram comprising a weight loss corresponding to 0.5 moles of water.
A.34 Any of Crystalline Form A or A.1-A.33, wherein the Crystalline Form A exhibits a thermogravimetric analysis (TGA) thermogram as shown in FIG. 4 and/or FIG. 5.
A.35 Any of Crystalline Form A or A.1-A.34, wherein the Crystalline Form A exhibits a dynamic (water) vapor sorption (DVS) isotherm comprising 3% or less weight gain upon increasing the relative humidity from 5% to 95%, e.g., 2.5% or less weight gain upon increasing the relative humidity from 5% to 95%.
A.36 Any of Crystalline Form A or A.1-A.35, wherein the Crystalline Form A exhibits a dynamic (water) vapor sorption (DVS) isotherm as shown in FIG. 6. For instance, any of Crystalline Form A or A.1-A.35, wherein the Crystalline Form A remains unchanged by XRPD after a dynamic (water) vapor sorption (DVS) experiment.
A.37 Any of Crystalline Form A or A.1-A.36, wherein the Crystalline Form A exhibits an FT-Raman spectrum comprising peaks (cm⁻¹) of 1618, 1539, 1529, and 1417. For instance, any of Crystalline Form A or A.1-A.36, wherein the Crystalline Form A exhibits an FT-Raman spectrum comprising peaks (cm⁻¹) of 2992, 1618, 1539, 1529, 1417, 1388, 1331, 1311, 1267, 1203, 1159, 1114, 1021, 983, and 965. For instance, any of Crystalline Form A or A.1-A.36, wherein the Crystalline Form A exhibits an FT-Raman spectrum comprising peaks (cm⁻¹) of 2992, 2920, 1618, 1539, 1529, 1417, 1388, 1331, 1311, 1267, 1203, 1159, 1114, 1021, 983, and 965.
A.38 Any of Crystalline Form A or A.1-A.37, wherein the Crystalline Form A exhibits characteristic Raman peaks (cm⁻¹) selected from those set forth in Crystalline Form A.37.
A.39 Any of Crystalline Form A or A.1-A.38, wherein the Crystalline Form A is a hydrate. For instance, any of Crystalline Form A or A.1-A.38, wherein the Crystalline Form A comprises about 0.5 moles of water (e.g., hemi-hydrate).
A.40 Any of Crystalline Form A or A.1-A.39, wherein the Crystalline Form A does not exhibit a melting point based on DSC data.
A.41 Any of Crystalline Form A or A.1-A.40, wherein the Crystalline Form A is made as described in any of Process 1 or 1.1 et seq. vide infra or as described in any of Process 3 or 3.1 et seq. vide infra.
A.42 Any of Crystalline Form A or A.1-A.41, wherein the Crystalline Form A is made as described in any of the examples that produce Crystalline Form A.
A.43 Any of Crystalline Form A or A.1-A.42, wherein any of the listed 2-theta (°) values of an XRPD pattern have an acceptable deviation of ±0.2°.
A.44 Any of Crystalline Form A or A.1-A.43, wherein the 2-theta (°) values of an XRPD pattern have an acceptable deviation of ±0.2°.
A.45 Any of Crystalline Form A or A.1-A.44, wherein the Crystalline Form A is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of the amorphous form.
A.46 Any of Crystalline Form A or A.1-A.45, wherein the Crystalline Form A is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of any other crystalline form.
A.47 Any of Crystalline Form A or A.1-A.46, wherein the Crystalline Form A is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of the amorphous form and any other crystalline form.
A.48 Any of Crystalline Form A or A.1-A.47, wherein the Crystalline Form A is formulated as described in any of Process 2 or 2.1 et seq. vide infra.
A.49 Any of Crystalline Form A, A.1-A.44, or A.48, wherein the Crystalline Form A is in a mixture with another crystalline form and/or amorphous form of trans sodium crocetinate. For instance, any of Crystalline Form A, A.1-A.44, or A.48, wherein the Crystalline Form A is in a mixture with any of Crystalline Form F or F.1 et seq. vide infra.
A.50 Any of Crystalline Form A or A.1-A.49, wherein the Crystalline Form A remains unchanged after DVS analysis where the sample is exposed up to 95% relative humidity (RH) and down to 5% RH and/or remains unchanged when heated to 100° C. for one minute and/or and remains unchanged for up to 4 weeks at 25° C./60% relative humidity (RH) and/or up to 4 weeks at 40° C./75% RH.
A.51 Any of Crystalline Form A or A.1-A.50, wherein the Crystalline Form A is ≥96% w/w pure by HPLC, e.g., ≥97% w/w pure by HPLC, e.g., ≥98% w/w pure by HPLC, e.g., ≥99% w/w pure by HPLC.

Also provided is Crystalline Form B (also called Form B herein) of trans sodium crocetinate (Formula I above). Further provided is Crystalline Form B as follows:
B.1 Crystalline Form B, wherein the Crystalline Form B exhibits an XRPD pattern comprising 2-theta (°) values 4.1, 8.0, and 16.6, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. Or, for instance, Crystalline Form B, wherein the Crystalline Form B exhibits an XRPD pattern comprising d-spacing (Å) values of 21.7, 11.0, and 5.3.
B.2 Crystalline Form B or B.1, wherein the Crystalline Form B exhibits an XRPD pattern comprising 2-theta (°) values of 4.1, 8.0, 16.6, 17.3, and 18.9, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. Or, for instance, Crystalline Form B or B.1, wherein the Crystalline Form B exhibits an XRPD pattern comprising d-spacing (Å) values of 21.7, 11.0, 5.3, 5.1, and 4.7.
B.3 Any of Crystalline Form B, B.1, or B.2, wherein the Crystalline Form B exhibits an XRPD pattern comprising at least three, e.g., at least five, e.g., at least ten, e.g., at least fifteen, e.g., at least twenty, e.g., at least twenty-five, 2-theta (°) values selected from the group consisting of 4.1, 8.0, 16.6, 17.3, 18.9, 20.0, 21.9, 22.5, 23.2, 23.9, 24.1, 24.3, 25.0, 26.0, 27.8, 28.0, 28.8, 29.2, 30.2, 30.4, 30.8, 31.6, 31.9, 33.6, 34.1, 35.1, 36.4, 37.7, and 38.2, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. For instance, Crystalline Form B, B.1, or B.2, wherein the Crystalline Form B exhibits an XRPD pattern comprising at least three, e.g., at least five, e.g., at least ten, e.g., at least fifteen, 2-theta (°) values selected from the group consisting of 4.1, 8.0, 16.6, 17.3, 18.9, 23.2, 23.9, 24.1, 24.3, 25.0, 27.8, 28.0, 28.8, 31.6, 31.9, 33.6, 34.1, 35.1, and 36.4, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
B.4 Any of Crystalline Form B or B.1-B.3, wherein the Crystalline Form B exhibits an XRPD pattern comprising 2-theta (°) values of 4.1, 8.0, 16.6, 17.3, 18.9, 23.2, 23.9, 24.1, 24.3, 25.0, 27.8, 28.0, 28.8, 31.6, 31.9, 33.6, 34.1, 35.1, and 36.4, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. For instance any Crystalline Form B or B.1-B.3, wherein the Crystalline Form B exhibits an XRPD pattern comprising 2-theta (°) values of 4.1, 8.0, 16.6, 17.3, 18.9, 20.0, 21.9, 22.5, 23.2, 23.9, 24.1, 24.3, 25.0, 26.0, 27.8, 28.0, 28.8, 29.2, 30.2, 30.4, 30.8, 31.6, 31.9, 33.6, 34.1, 35.1, 36.4, 37.7, and 38.2, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
B.5 Any of Crystalline Form B or B.1-B.4, wherein the Crystalline Form B exhibits an XRPD pattern comprising at least three, e.g., at least five, e.g., at least ten, e.g., at least fifteen, e.g., at least twenty, e.g., at least twenty-five, 2-theta (°) values selected from the group consisting of 4.07, 8.04, 16.56, 17.26, 18.91, 19.95, 21.92, 22.49, 23.21, 23.88, 24.09, 24.34, 24.97, 26.02, 27.83, 28.01, 28.84, 29.15, 30.17, 30.40, 30.83, 31.56, 31.94, 33.56, 34.08, 35.05, 36.37, 37.65, and 38.17, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
B.6 Any of Crystalline Form B or B.1-B.5, wherein the Crystalline Form B exhibits an XRPD pattern comprising 2-theta (°) values of 4.07, 8.04, 16.56, 17.26, 18.91, 19.95, 21.92, 22.49, 23.21, 23.88, 24.09, 24.34, 24.97, 26.02, 27.83, 28.01, 28.84, 29.15, 30.17, 30.40, 30.83, 31.56, 31.94, 33.56, 34.08, 35.05, 36.37, 37.65, and 38.17, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
B.7 Any of Crystalline Form B or B.1-B.6, wherein the Crystalline Form B exhibits an XRPD pattern comprising at least three, e.g., at least five, e.g., at least ten, e.g., at least fifteen, e.g., at least twenty, d-spacing (Å) values selected from the group consisting of 21.7, 11.0, 5.3, 5.1, 4.7, 4.4, 4.1, 4.0, 3.8, 3.7, 3.6, 3.4, 3.2, 3.1, 3.0, 2.9, 2.8, 2.7, 2.6, 2.5, and 2.4. For instance, any of Crystalline Form B or B.1-B.6, wherein the Crystalline Form B exhibits an XRPD pattern comprising at least three, e.g., at least five, e.g., at least ten, d-spacing (Å) values selected from the group consisting of 21.7, 11.0, 5.3, 5.1, 4.7, 3.8, 3.7, 3.6, 3.2, 3.1, 2.8, 2.7, 2.6, and 2.5.
B.8 Any of Crystalline Form B or B.1-B.7, wherein the Crystalline Form B exhibits an XRPD pattern comprising d-spacing (Å) values of 21.7, 11.0, 5.3, 5.1, 4.7, 3.8, 3.7, 3.6, 3.2, 3.1, 2.8, 2.7, 2.6, and 2.5. For instance, any of Crystalline Form B or B.1-B.7, wherein the Crystalline Form B exhibits an XRPD pattern comprising d-spacing (Å) values of 21.7, 11.0, 5.3, 5.1, 4.7, 4.4, 4.1, 4.0, 3.8, 3.7, 3.6, 3.4, 3.2, 3.1, 3.0, 2.9, 2.8, 2.7, 2.6, 2.5, and 2.4.
B.9 Any of Crystalline Form B or B.1-B.8, wherein the Crystalline Form B exhibits an XRPD pattern comprising at least three, e.g., at least five, e.g., at least ten, e.g., at least fifteen, e.g., at least twenty, e.g., at least twenty-five, d-spacing (Å) values selected from the group consisting of 21.69, 10.99, 5.35, 5.13, 4.69, 4.45, 4.05, 3.95, 3.83, 3.72, 3.69, 3.65, 3.56, 3.42, 3.20, 3.18, 3.09, 3.06, 2.96, 2.94, 2.90, 2.83, 2.80, 2.67, 2.63, 2.56, 2.47, 2.39, and 2.36. For instance, any of Crystalline Form B or B.1-B.8, wherein the Crystalline Form B exhibits an XRPD pattern comprising at least three, e.g., at least five, e.g., at least ten, e.g., at least fifteen, d-spacing (Å) values selected from the group consisting of 21.69, 10.99, 5.35, 5.13, 4.69, 3.83, 3.72, 3.69, 3.65, 3.56, 3.20, 3.18, 3.09, 2.83, 2.80, 2.67, 2.63, 2.56, and 2.47.
B.10 Any of Crystalline Form B or B.1-B.9, wherein the Crystalline Form B exhibits an XRPD pattern comprising d-spacing (N) values of 21.69, 10.99, 5.35, 5.13, 4.69, 3.83, 3.72, 3.69, 3.65, 3.56, 3.20, 3.18, 3.09, 2.83, 2.80, 2.67, 2.63, 2.56, and 2.47. For instance, any of Crystalline Form B or B.1-B.9, wherein the Crystalline Form B exhibits an XRPD pattern comprising d-spacing (Å) values of 21.69, 10.99, 5.35, 5.13, 4.69, 4.45, 4.05, 3.95, 3.83, 3.72, 3.69, 3.65, 3.56, 3.42, 3.20, 3.18, 3.09, 3.06, 2.96, 2.94, 2.90, 2.83, 2.80, 2.67, 2.63, 2.56, 2.47, 2.39, and 2.36.
B.11 Any of Crystalline Form B or B.1-B.10, wherein the Crystalline Form B exhibits an XRPD pattern comprising at least three, e.g., at least five, e.g., at least ten, e.g., at least fifteen, e.g., at least twenty, e.g., at least twenty-five, d-spacing (Å) values selected from the group consisting of 21.686, 10.991, 5.349, 5.134, 4.689, 4.446, 4.051, 3.951, 3.829, 3.723, 3.692, 3.655, 3.564, 3.422, 3.204, 3.183, 3.093, 3.061, 2.960, 2.938, 2.898, 2.833, 2.800, 2.668, 2.628, 2.558, 2.468, 2.387, and 2.356. For instance, any of Crystalline Form B or B.1-B.10, wherein the Crystalline Form B exhibits an XRPD pattern comprising at least three, e.g., at least five, e.g., at least ten, e.g., at least fifteen, d-spacing (Å) values selected from the group consisting of 21.686, 10.991, 5.349, 5.134, 4.689, 3.829, 3.723, 3.692, 3.655, 3.564, 3.204, 3.183, 3.093, 2.833, 2.800, 2.668, 2.628, 2.558, and 2.468.
B.12 Any of Crystalline Form B or B.1-B.11, wherein the Crystalline Form B exhibits an XRPD pattern comprising d-spacing (_) values of 21.686, 10.991, 5.349, 5.134, 4.689, 3.829, 3.723, 3.692, 3.655, 3.564, 3.204, 3.183, 3.093, 2.833, 2.800, 2.668, 2.628, 2.558, and 2.468. For instance, any of Crystalline Form B or B.1-B.11, wherein the Crystalline Form B exhibits an XRPD pattern comprising d-spacing (Å) values of 21.686, 10.991, 5.349, 5.134, 4.689, 4.446, 4.051, 3.951, 3.829, 3.723, 3.692, 3.655, 3.564, 3.422, 3.204, 3.183, 3.093, 3.061, 2.960, 2.938, 2.898, 2.833, 2.800, 2.668, 2.628, 2.558, 2.468, 2.387, and 2.356.
B.13 Any of Crystalline Form B or B.1-B.12, wherein the Crystalline Form B exhibits an XRPD pattern comprising at least three, e.g., at least five, e.g., at least ten, e.g., at least fifteen, e.g., at least twenty, e.g., at least twenty-five, e.g., at least thirty, 2-theta (°) values selected from those set forth in Table B below:

TABLE B
Form B

| Peak Position | Relative | d-spacing |
|---|---|---|
| (°2θ)^{a} | Intensity | value (Å) |
| 4.07 | 29.84 | 21.6864 |
| 8.04 | 9.99 | 10.9909 |
| 16.56 | 4.52 | 5.3491 |
| 17.26 | 30.61 | 5.1339 |
| 18.91 | 8.01 | 4.6886 |
| 19.95 | 1.04 | 4.4461 |
| 21.92 | 1.66 | 4.0510 |
| 22.49 | 1.02 | 3.9505 |
| 23.21 | 8.83 | 3.8294 |
| 23.88 | 100.00 | 3.7229 |
| 24.09 | 26.17 | 3.6919 |
| 24.34 | 57.20 | 3.6546 |
| 24.97 | 6.67 | 3.5635 |
| 26.02 | 1.17 | 3.4219 |
| 27.83 | 3.23 | 3.2035 |
| 28.01 | 3.19 | 3.1825 |
| 28.84 | 15.22 | 3.0928 |
| 29.15 | 2.28 | 3.0609 |
| 30.17 | 1.32 | 2.9596 |
| 30.40 | 1.77 | 2.9382 |
| 30.83 | 1.43 | 2.8976 |
| 31.56 | 2.95 | 2.8326 |
| 31.94 | 2.01 | 2.7998 |
| 33.56 | 14.29 | 2.6680 |
| 34.08 | 19.33 | 2.6284 |
| 35.05 | 6.76 | 2.5581 |
| 36.37 | 5.31 | 2.4683 |
| 37.65 | 2.11 | 2.3869 |
| 38.17 | 1.28 | 2.3557, |

^{a}+/−0.2°2θ per USP guidelines

e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
B.14 Any of Crystalline Form B or B.1-B.13, wherein the Crystalline Form B exhibits an XRPD pattern comprising the 2-theta (°) values set forth in Table B in Crystalline Form B.13, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
B.15 Any of Crystalline Form B or B.1-B.14, wherein the Crystalline Form B exhibits an XRPD pattern comprising at least three, e.g., at least five, e.g., at least ten, e.g., at least fifteen, e.g., at least twenty, e.g., at least twenty-five, e.g., at least thirty, d-spacing (Å) values selected from those set forth in Table B in Crystalline Form B.13.
B.16 Any of Crystalline Form B or B.1-B.15, wherein the Crystalline Form B exhibits an XRPD pattern comprising the d-spacing (Å) values set forth in Table B in Crystalline Form B.13.
B.17 Any of Crystalline Form B or B.1-B.16, wherein the Crystalline Form B exhibits an XRPD pattern comprising at least four, e.g., at least five, e.g., at least ten, e.g., at least fifteen, 2-theta (°) values selected from those set forth in Table BB below:

TABLE BB

| | Relative | d-spacing |
|---|---|---|
| °2θ | Intensity | value (Å) |
| 4.1 | 30 | 21.6864 |
| 8.0 | 10 | 10.9909 |
| 16.6 | 5 | 5.3491 |
| 17.3 | 31 | 5.1339 |
| 18.9 | 8 | 4.6886 |
| 23.2 | 9 | 3.8294 |
| 23.9 | 100 | 3.7229 |
| 24.1 | 26 | 3.6919 |
| 24.3 | 57 | 3.6546 |
| 25.0 | 7 | 3.5635 |
| 27.8 | 3 | 3.2035 |
| 28.0 | 3 | 3.1825 |
| 28.8 | 15 | 3.0928 |
| 31.6 | 3 | 2.8326 |
| 31.9 | 2 | 2.7998 |
| 33.6 | 14 | 2.6680 |
| 34.1 | 19 | 2.6284 |
| 35.1 | 7 | 2.5581 |
| 36.4 | 5 | 2.4683 |
| — | — | —, |

e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
B.18 Any of Crystalline Form B or B.1-B.17, wherein the Crystalline Form B exhibits an XRPD pattern comprising the 2-theta (°) values set forth in Table BB in Crystalline Form B.17, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
B.19 Any of Crystalline Form B or B.1-B.18, wherein the Crystalline Form B exhibits an XRPD pattern comprising at least four, e.g., at least five, e.g., at least ten, e.g., at least fifteen, e.g., at least twenty, d-spacing (Å) values selected from those set forth in Table BB in Crystalline Form B.17.
B.20 Any of Crystalline Form B or B.1-B.19, wherein the Crystalline Form B exhibits an XRPD pattern comprising the d-spacing (Å) values set forth in Table BB in Crystalline Form B.17.
B.21 Any of Crystalline Form B or B.1-B.20, wherein the Crystalline Form B exhibits an XRPD pattern comprising characteristic 2-theta (°) values selected from any of the 2-theta (°) values set forth in any of Crystalline Form B or B.1-B.20 above, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
B.22 Any of Crystalline Form B or B.1-B.21, wherein the Crystalline Form B exhibits an XRPD pattern comprising representative 2-theta (°) values selected from any of the 2-theta (°) values set forth in any of Crystalline Form B or B.1-B.20 above, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
B.23 Any of Crystalline Form B or B.1-B.22, wherein the Crystalline Form B exhibits an XRPD pattern comprising characteristic d-spacing (Å) values selected from any of the d-spacing (Å) values set forth in any of Crystalline Form B or B.1-B.20 above.
B.24 Any of Crystalline Form B or B.1-B.23, wherein the Crystalline Form B exhibits an XRPD pattern comprising representative d-spacing (Å) values selected from any of the d-spacing (Å) values set forth in any of Crystalline Form B or B.1-B.20 above.
B.25 Any of Crystalline Form B or B.1-B.24, wherein the Crystalline Form B exhibits an XRPD pattern comprising at least three, e.g., at least five, e.g., at least ten, e.g., at least fifteen, e.g., at least twenty, e.g., at least twenty-five, e.g., at least thirty, e.g., all, of the peaks of the XRPD shown in FIG. 7, FIG. 8, FIG. 13, FIG. 30, FIG. 32, and/or FIG. 34, e.g., of the XRPD shown in FIG. 7, FIG. 8, FIG. 30, FIG. 32, and/or FIG. 34, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. FIG. 9 is the same as FIG. 8, FIG. 31 is the same XRPD as FIG. 30, FIG. 33 is the same XRPD as FIG. 32, and FIG. 35 is the same XRPD as FIG. 34, but with different y-axis scales.
B.26 Any of Crystalline Form B or B.1-B.25, wherein the Crystalline Form B exhibits an XRPD pattern comprising the characteristic peaks of the XRPD shown in FIG. 7, FIG. 8, FIG. 13, FIG. 30, FIG. 32, and/or FIG. 34, e.g., of the XRPD shown in FIG. 7, FIG. 8, FIG. 30, FIG. 32, and/or FIG. 34, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
B.27 Any of Crystalline Form B or B.1-B.26, wherein the Crystalline Form B exhibits an XRPD pattern comprising the representative peaks of the XRPD shown in FIG. 7, FIG. 8, FIG. 13, FIG. 30, FIG. 32, and/or FIG. 34, e.g., of the XRPD shown in FIG. 7, FIG. 8, FIG. 30, FIG. 32, and/or FIG. 34, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
B.28 Any of Crystalline Form B or B.1-B.27, wherein the Crystalline Form B exhibits an XRPD corresponding to FIG. 7, FIG. 8, FIG. 13, FIG. 30, FIG. 32, and/or FIG. 34, e.g., corresponding to FIG. 7, FIG. 8, FIG. 30, FIG. 32, and/or FIG. 34, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
B.29 Any of Crystalline Form B or B.1-B.28, wherein the Crystalline Form B exhibits a differential scanning calorimetry (DSC) thermogram comprising an endotherm between 90-100° C., e.g., between 90-95° C. For instance, any of Crystalline Form B or B.1-B.22, wherein the Crystalline Form B exhibits a differential scanning calorimetry (DSC) thermogram comprising an endotherm at 92° C. (e.g., 92.4° C.).
B.30 Any of Crystalline Form B or B.1-B.29, wherein the Crystalline Form B exhibits a differential scanning calorimetry (DSC) thermogram comprising an endotherm between 110-120° C., e.g., between 110-115° C. For instance, any of Crystalline Form B or B.1-B.29, wherein the Crystalline Form B exhibits a differential scanning calorimetry (DSC) thermogram comprising an endotherm at 113° C. (e.g., 113.3° C.).
B.31 Any of Crystalline Form B or B.1-B.30, wherein the Crystalline Form B exhibits a differential scanning calorimetry (DSC) thermogram as shown in FIG. 10.
B.32 Any of Crystalline Form B or B.1-B.31, wherein the Crystalline Form B exhibits a thermogravimetric analysis (TGA) thermogram comprising weight loss between start (e.g., 25° C.) and 100° C., e.g., between start (e.g., 25° C.) and 95° C., e.g., a weight loss of 4-5 weight %, e.g., a weight loss of 4.6 weight %.
B.33 Any of Crystalline Form B or B.1-B.32, wherein the Crystalline Form B exhibits a thermogravimetric analysis (TGA) thermogram comprising a weight loss corresponding to 1 mole of water.
B.34 Any of Crystalline Form B or B.1-B.33, wherein the Crystalline Form B exhibits a thermogravimetric analysis (TGA) thermogram as shown in FIG. 11.
B.35 Any of Crystalline Form B or B.1-B.34, wherein the Crystalline Form B is a hydrate. For instance, any of Crystalline Form B or B.1-B.34, wherein the Crystalline Form B comprises about 1 mole of water (e.g., monohydrate).
B.36 Any of Crystalline Form B or B.1-B.36, wherein the Crystalline Form B exhibits an FT-Raman spectrum comprising peaks (cm⁻¹) of 1619, 1534, and 1408. For instance, any of Crystalline Form B or B.1-B.36, wherein the Crystalline Form B exhibits an FT-Raman spectrum comprising peaks (cm⁻¹) of 2920, 1619, 1534, 1447, 1408, 1351, 1333, 1266, 1203, 1161, 1114, 1018, 979, and 894.
B.37 Any of Crystalline Form B or B.1-B.36, wherein the Crystalline Form B exhibits characteristic Raman peaks (cm⁻¹) selected from those set forth in Crystalline Form B.36.
B.38 Any of Crystalline Form B or B.1-B.37, wherein the Crystalline Form B does not exhibit a melting point based on DSC data.
B.39 Any of Crystalline Form B or B.1-B.38, wherein the Crystalline Form B is made as described in any of Process 1 or 1.1 et seq. vide infra or as described in any of Process 3 or 3.1 et seq. vide infra.
B.40 Any of Crystalline Form B or B.1-B.39, wherein the Crystalline Form B is made as described in any of the examples that produce Crystalline Form B.
B.41 Any of Crystalline Form B or B.1-B.40, wherein any of the listed 2-theta (°) values of an XRPD pattern have an acceptable deviation of ±0.2°.
B.42 Any of Crystalline Form B or B.1-B.41, wherein the 2-theta (°) values of an XRPD pattern have an acceptable deviation of ±0.2°.
B.43 Any of Crystalline Form B or B.1-B.42, wherein the Crystalline Form B is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of the amorphous form.
B.44 Any of Crystalline Form B or B.1-B.43, wherein the Crystalline Form B is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of any other crystalline form.
B.45 Any of Crystalline Form B or B.1-B.44, wherein the Crystalline Form B is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of the amorphous form and any other crystalline form.
B.46 Any of Crystalline Form B or B.1-B.45, wherein the Crystalline Form B is formulated as described in any of Process 2 or 2.1 et seq. vide infra
B.47 Any of Crystalline Form B, B.1-B.42, or B.46, wherein the Crystalline Form B is in a mixture with another crystalline form and/or amorphous form of trans sodium crocetinate. For instance, any of Crystalline Form B, B.1-B.42, or B.46, wherein the Crystalline Form B is in a mixture with any of Crystalline Form F or F.1 et seq. vide infra.
B.48 Any of Crystalline Form B or B.1-B.47, wherein the Crystalline Form B is ≥96% w/w pure by HPLC, e.g., ≥97% w/w pure by HPLC, e.g., ≥98% w/w pure by HPLC, e.g., ≥99% w/w pure by HPLC.

Also provided is Crystalline Form C (also called Form C herein) of trans sodium crocetinate (Formula I above). Further provided is Crystalline Form C as follows:
C.1 Crystalline Form C, wherein the Crystalline Form C exhibits an XRPD pattern comprising 2-theta (°) values 3.5, 3.8, 6.9, and 10.4, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. Or, for instance, Crystalline Form C, wherein the Crystalline Form C exhibits an XRPD pattern comprising d-spacing (Å) values of 25.4, 23.3, 12.8, and 8.5.
C.2 Crystalline Form C or C.1, wherein the Crystalline Form C exhibits an XRPD pattern comprising 2-theta (°) values of 3.5, 3.8, 6.9, 10.4, 11.3, and 13.8, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. Or, for instance, Crystalline Form C or C.1, wherein the Crystalline Form C exhibits an XRPD pattern comprising d-spacing (Å) values of 25.4, 23.3, 12.8, 8.5, 7.8, and 6.4.
C.3 Any of Crystalline Form C, C.1, or C.2, wherein the Crystalline Form C exhibits an XRPD pattern comprising at least three, e.g., at least four, e.g., at least five, e.g., at least six, e.g., at least ten, 2-theta (°) values selected from the group consisting of 3.5, 3.8, 4.3, 6.9, 10.4, 11.3, 13.8, 17.3, 18.9, 24.3, 26.6, 27.8, and 34.9, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
C.4 Any of Crystalline Form C or C.1-C.3, wherein the Crystalline Form C exhibits an XRPD pattern comprising 2-theta (°) values of 3.5, 3.8, 4.3, 6.9, 10.4, 11.3, 13.8, 17.3, 18.9, 24.3, 26.6, 27.8, and 34.9, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
C.5 Any of Crystalline Form C or C.1-C.4, wherein the Crystalline Form C exhibits an XRPD pattern comprising at least three, e.g., at least four, e.g., at least five, e.g., at least six, e.g., at least ten, 2-theta (°) values selected from the group consisting of 3.48, 3.79, 4.25, 6.93, 10.37, 11.31, 13.82, 17.29, 18.87, 24.26, 26.61, 27.79, and 34.94, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
C.6 Any of Crystalline Form C or C.1-C.5, wherein the Crystalline Form C exhibits an XRPD pattern comprising 2-theta (°) values of 3.48, 3.79, 4.25, 6.93, 10.37, 11.31, 13.82, 17.29, 18.87, 24.26, 26.61, 27.79, and 34.94, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
C.7 Any of Crystalline Form C or C.1-C.6, wherein the Crystalline Form C exhibits an XRPD pattern comprising at least three, e.g., at least four, e.g., at least five, e.g., at least six, e.g., at least ten, d-spacing (Å) values selected from the group consisting of 25.4, 23.3, 20.8, 12.8, 8.5, 7.8, 6.4, 5.1, 4.7, 3.7, 3.3, 3.2, and 2.6.
C.8 Any of Crystalline Form C or C.1-C.7, wherein the Crystalline Form C exhibits an XRPD pattern comprising d-spacing (Å) values of 25.4, 23.3, 20.8, 12.8, 8.5, 7.8, 6.4, 5.1, 4.7, 3.7, 3.3, 3.2, and 2.6.
C.9 Any of Crystalline Form C or C.1-C.8, wherein the Crystalline Form C exhibits an XRPD pattern comprising at least three, e.g., at least four, e.g., at least five, e.g., at least six, e.g., at least ten, d-spacing (Å) values selected from the group consisting of 25.37, 23.30, 20.80, 12.75, 8.53, 7.82, 6.40, 5.13, 4.70, 3.67, 3.35, 3.21, and 2.57.
C.10 Any of Crystalline Form C or C.1-C.9, wherein the Crystalline Form C exhibits an XRPD pattern comprising d-spacing (Å) values of 25.37, 23.30, 20.80, 12.75, 8.53, 7.82, 6.40, 5.13, 4.70, 3.67, 3.35, 3.21, and 2.57.
C.11 Any of Crystalline Form C or C.1-C.10, wherein the Crystalline Form C exhibits an XRPD pattern comprising at least three, e.g., at least four, e.g., at least five, e.g., at least six, e.g., at least ten, d-spacing (Å) values selected from the group consisting of 25.370, 23.300, 20.799, 12.751, 8.526, 7.819, 6.403, 5.126, 4.699, 3.666, 3.347, 3.208, and 2.566.
C.12 Any of Crystalline Form C or C.1-C.11, wherein the Crystalline Form C exhibits an XRPD pattern comprising d-spacing (Å) values of 25.370, 23.300, 20.799, 12.751, 8.526, 7.819, 6.403, 5.126, 4.699, 3.666, 3.347, 3.208, and 2.566.
C.13 Any of Crystalline Form C or C.1-C.12, wherein the Crystalline Form C exhibits an XRPD pattern comprising at least three, e.g., at least four, e.g., at least five, e.g., at least six, e.g., at least ten, 2-theta (°) values selected from those set forth in Table C below:

TABLE C
Form C

| Peak Position | Relative | d-spacing |
|---|---|---|
| (°2θ)^{a} | Intensity | value (Å) |
| 3.48 | 100.00 | 25.3701 |
| 3.79 | 9.20 | 23.3002 |
| 4.25 | 2.73 | 20.7986 |
| 6.93 | 7.29 | 12.7507 |
| 10.37 | 7.61 | 8.5255 |
| 11.31 | 1.03 | 7.8190 |
| 13.82 | 8.56 | 6.4028 |
| 17.29 | 11.55 | 5.1259 |
| 18.87 | 1.90 | 4.6993 |
| 24.26 | 1.75 | 3.6661 |
| 26.61 | 1.06 | 3.3471 |
| 27.79 | 2.84 | 3.2075 |
| 34.94 | 2.01 | 2.5657, |

^{a}+/−0.2°2θ per USP guidelines

e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
C.14 Any of Crystalline Form C or C.1-C.13, wherein the Crystalline Form C exhibits an XRPD pattern comprising the 2-theta (°) values set forth in Table C in Crystalline Form C.13, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
C.15 Any of Crystalline Form C or C.1-C.14, wherein the Crystalline Form C exhibits an XRPD pattern comprising at least three, e.g., at least four, e.g., at least five, e.g., at least six, e.g., at least ten, d-spacing (Å) values selected from those set forth in Table C in Crystalline Form C.13.
C.16 Any of Crystalline Form C or C.1-C.15, wherein the Crystalline Form C exhibits an XRPD pattern comprising the d-spacing (Å) values set forth in Table C in Crystalline Form C.13.
C.17 Any of Crystalline Form C or C.1-C.16, wherein the Crystalline Form C exhibits an XRPD pattern comprising at least four, e.g., at least six, e.g., at least ten, 2-theta (°) values selected from those set forth in Table CC below:

TABLE CC

| | Relative | d-spacing |
|---|---|---|
| °2θ | Intensity | value (A) |
| 3.5 | 100 | 25.3701 |
| 3.8 | 9 | 23.3002 |
| 4.3 | 3 | 20.7986 |
| 6.9 | 7 | 12.7507 |
| 10.4 | 8 | 8.5255 |
| 11.3 | 1 | 7.8190 |
| 13.8 | 9 | 6.4028 |
| 17.3 | 12 | 5.1259 |
| 18.9 | 2 | 4.6993 |
| 24.3 | 2 | 3.6661 |
| 26.6 | 1 | 3.3471 |
| 27.8 | 3 | 3.2075 |
| 34.9 | 2 | 2.5657, |

e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
C.18 Any of Crystalline Form C or C.1-C.17, wherein the Crystalline Form C exhibits an XRPD pattern comprising the 2-theta (°) values set forth in Table CC in Crystalline Form C.17, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
C.19 Any of Crystalline Form C or C.1-C.18, wherein the Crystalline Form C exhibits an XRPD pattern comprising at least four, e.g., at least six, e.g., at least ten, d-spacing (Å) values selected from those set forth in Table CC in Crystalline Form C.17.
C.20 Any of Crystalline Form C or C.1-C.19, wherein the Crystalline Form C exhibits an XRPD pattern comprising the d-spacing (Å) values set forth in Table CC in Crystalline Form C.17.
C.21 Any of Crystalline Form C or C.1-C.20, wherein the Crystalline Form C exhibits an XRPD pattern comprising characteristic 2-theta (°) values selected from any of the 2-theta (°) values set forth in any of Crystalline Form C or C.1-C.20 above, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
C.22 Any of Crystalline Form C or C.1-C.21, wherein the Crystalline Form C exhibits an XRPD pattern comprising representative 2-theta (°) values selected from any of the 2-theta (°) values set forth in any of Crystalline Form C or C.1-C.21 above, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
C.23 Any of Crystalline Form C or C.1-C.22, wherein the Crystalline Form C exhibits an XRPD pattern comprising characteristic d-spacing (Å) values selected from any of the d-spacing (Å) values set forth in any of Crystalline Form C or C.1-C.20 above.
C.24 Any of Crystalline Form C or C.1-C.23, wherein the Crystalline Form C exhibits an XRPD pattern comprising representative d-spacing (Å) values selected from any of the d-spacing (Å) values set forth in any of Crystalline Form C or C.1-C.20 above.
C.25 Any of Crystalline Form C or C.1-C.24, wherein the Crystalline Form C exhibits an XRPD pattern comprising at least three, e.g., at least five, e.g., at least ten, e.g., at least fifteen, e.g., at least twenty, e.g., at least twenty-five, e.g., at least thirty, e.g., all, of the peaks of the XRPD shown in FIG. 14, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. FIG. 15 is the same as FIG. 14, but with different y-axis scales.
C.26 Any of Crystalline Form C or C.1-C.25, wherein the Crystalline Form C exhibits an XRPD pattern comprising the characteristic peaks of the XRPD shown in FIG. 14, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
C.27 Any of Crystalline Form C or C.1-C.26, wherein the Crystalline Form C exhibits an XRPD pattern comprising the representative peaks of the XRPD shown in FIG. 14, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
C.28 Any of Crystalline Form C or C.1-C.27, wherein the Crystalline Form C exhibits an XRPD corresponding to FIG. 14, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
C.29 Any of Crystalline Form C or C.1-C.28, wherein the Crystalline Form C exhibits a differential scanning calorimetry (DSC) thermogram comprising an endotherm between 80-90° C., e.g., between 80-85° C. For instance, any of Crystalline Form C or C.1-C.28, wherein the Crystalline Form C exhibits a differential scanning calorimetry (DSC) thermogram comprising an endotherm at 81° C. (e.g., 81.3° C.).
C.30 Any of Crystalline Form C or C.1-C.29, wherein the Crystalline Form C exhibits a differential scanning calorimetry (DSC) thermogram comprising an endotherm between 130-140° C., e.g., between 130-135° C. For instance, any of Crystalline Form C or C.1-C.29, wherein the Crystalline Form C exhibits a differential scanning calorimetry (DSC) thermogram comprising an endotherm at 133° C. (e.g., 132.6° C.).
C.31 Any of Crystalline Form C or C.1-C.30, wherein the Crystalline Form C exhibits a differential scanning calorimetry (DSC) thermogram as shown in FIG. 16.
C.32 Any of Crystalline Form C or C.1-C.31, wherein the Crystalline Form C exhibits a thermogravimetric analysis (TGA) thermogram comprising weight loss between start (e.g., 25° C.) and 130° C., e.g., between start (e.g., 25° C.) and 125° C., e.g., a weight loss of 14-15 weight %, e.g., a weight loss of 14.3 weight %.
C.33 Any of Crystalline Form C or C.1-C.32, wherein the Crystalline Form C exhibits a thermogravimetric analysis (TGA) thermogram comprising a weight loss corresponding to at least 3 moles of water, e.g., corresponding to 3.5 moles of water.
C.34 Any of Crystalline Form C or C.1-C.33, wherein the Crystalline Form C exhibits a thermogravimetric analysis (TGA) thermogram as shown in FIG. 17.
C.35 Any of Crystalline Form C or C.1-C.34, wherein the Crystalline Form C is a hydrate. For instance, any of Crystalline Form C or C.1-C.34, wherein the Crystalline Form C comprises at least 3 moles of water, e.g., about 3.5 moles of water.
C.36 Any of Crystalline Form C or C.1-C.35, wherein the Crystalline Form C exhibits an FT-Raman spectrum comprising peaks (cm⁻¹) of 1622, 1537, 1396, and 1343. Any of Crystalline Form C or C.1-C.35, wherein the Crystalline Form C exhibits an FT-Raman spectrum comprising peaks (cm⁻¹) of 2920, 1622, 1537, 1443, 1396, 1343, 1261, 1209, 1168, 1117, 1019, and 965.
C.37 Any of Crystalline Form C or C.1-C.36, wherein the Crystalline Form C exhibits characteristic Raman peaks (cm⁻¹) selected from those set forth in Crystalline Form C.36.
C.38 Any of Crystalline Form C or C.1-C.37, wherein the Crystalline Form C does not exhibit a melting point based on DSC data.
C.39 Any of Crystalline Form C or C.1-C.38, wherein the Crystalline Form C is made as described in any of Process 1 or 1.1 et seq. vide infra or as described in any of Process 3 or 3.1 et seq. vide infra.
C.40 Any of Crystalline Form C or C.1-C.39, wherein the Crystalline Form C is made as described in any of the examples that produce Crystalline Form C.
C.41 Any of Crystalline Form C or C.1-C.40, wherein any of the listed 2-theta (°) values of an XRPD pattern have an acceptable deviation of ±0.2°.
C.42 Any of Crystalline Form C or C.1-C.41, wherein the 2-theta (°) values of an XRPD pattern have an acceptable deviation of ±0.2°.
C.43 Any of Crystalline Form C or C.1-C.42, wherein the Crystalline Form C is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of the amorphous form.
C.44 Any of Crystalline Form C or C.1-C.43, wherein the Crystalline Form C is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of any other crystalline form.
C.45 Any of Crystalline Form C or C.1-C.44, wherein the Crystalline Form C is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of the amorphous form and any other crystalline form.
C.46 Any of Crystalline Form C or C.1-C.45, wherein the Crystalline Form C is formulated as described in any of Process 2 or 2.1 et seq. vide infra.
C.47 Any of Crystalline Form C, C.1-C.42, or C.46, wherein the Crystalline Form C is in a mixture with another crystalline form and/or amorphous form of trans sodium crocetinate.
C.48 Any of Crystalline Form C or C.1-C.47, wherein the Crystalline Form C is ≥96% w/w pure by HPLC, e.g., ≥97% w/w pure by HPLC, e.g., ≥98% w/w pure by HPLC, e.g., ≥99% w/w pure by HPLC.

Also provided is Crystalline Form D (also called Form D herein) of trans sodium crocetinate (Formula I above). Further provided is Crystalline Form D as follows:
D.1 Crystalline Form D, wherein the Crystalline Form D exhibits an XRPD pattern comprising 2-theta (°) values 3.5, 4.3, and 8.4, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. Or, for instance, Crystalline Form D, wherein the Crystalline Form D exhibits an XRPD pattern comprising d-spacing (Å) values of 25.1, 20.8, and 10.5.
D.2 Crystalline Form D or D.1, wherein the Crystalline Form D exhibits an XRPD pattern comprising 2-theta (°) values of 3.5, 4.3, 8.4, 15.4, 16.1, and 19.5, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. Or, for instance, Crystalline Form D or D.1, wherein the Crystalline Form D exhibits an XRPD pattern comprising d-spacing (Å) values of 25.1, 20.8, 10.5, 5.8, 5.5, and 4.5.
D.3 Any of Crystalline Form D, D.1, or D.2, wherein the Crystalline Form D exhibits an XRPD pattern comprising at least three, e.g., at least five, e.g., at least six, e.g., at least ten, e.g., at least fifteen, e.g., at least twenty, e.g., at least twenty-five, e.g., at least thirty, 2-theta (°) values selected from the group consisting of 3.5, 4.1, 4.3, 7.0, 8.1, 8.4, 10.4, 13.1, 13.8, 15.4, 15.9, 16.1, 17.4, 19.0, 19.5, 22.7, 23.2, 23.4, 24.0, 25.2, 25.3, 25.9, 26.1, 28.8, 29.2, 29.6, 30.9, 31.9, 32.7, 34.0, 35.0, and 38.4, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. For instance, any of Crystalline Form D, D.1, or D.2, wherein the Crystalline Form D exhibits an XRPD pattern comprising at least three, e.g., at least five, e.g., at least six, e.g., at least ten, e.g., at least fifteen, 2-theta (°) values selected from the group consisting of 3.5, 4.1, 4.3, 7.0, 8.1, 8.4, 10.4, 13.1, 13.8, 15.4, 16.1, 17.4, 19.5, 22.7, 25.3, 25.9, 26.1, and 38.4, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
D.4 Any of Crystalline Form D or D.1-D.3, wherein the Crystalline Form D exhibits an XRPD pattern comprising 2-theta (°) values of 3.5, 4.1, 4.3, 7.0, 8.1, 8.4, 10.4, 13.1, 13.8, 15.4, 16.1, 17.4, 19.5, 22.7, 25.3, 25.9, 26.1, and 38.4, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. For instance, any of Crystalline Form D or D.1-D.3, wherein the Crystalline Form D exhibits an XRPD pattern comprising 2-theta (°) values of 3.5, 4.1, 4.3, 7.0, 8.1, 8.4, 10.4, 13.1, 13.8, 15.4, 15.9, 16.1, 17.4, 19.0, 19.5, 22.7, 23.2, 23.4, 24.0, 25.2, 25.3, 25.9, 26.1, 28.8, 29.2, 29.6, 30.9, 31.9, 32.7, 34.0, 35.0, and 38.4, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
D.5 Any of Crystalline Form D or D.1-D.4, wherein the Crystalline Form D exhibits an XRPD pattern comprising at least three, e.g., at least five, e.g., at least six, e.g., at least ten, e.g., at least fifteen, e.g., at least twenty, e.g., at least twenty-five, e.g., at least thirty, 2-theta (°) values selected from the group consisting of 3.52, 4.09, 4.25, 6.96, 8.05, 8.42, 10.40, 13.10, 13.84, 15.35, 15.88, 16.12, 17.38, 18.98, 19.51, 22.67, 23.15, 23.36, 23.98, 25.15, 25.32, 25.90, 26.06, 28.82, 29.21, 29.59, 30.94, 31.88, 32.67, 34.00, 35.04, and 38.37, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
D.6 Any of Crystalline Form D or D.1-D.5, wherein the Crystalline Form D exhibits an XRPD pattern comprising 2-theta (°) values of 3.52, 4.09, 4.25, 6.96, 8.05, 8.42, 10.40, 13.10, 13.84, 15.35, 15.88, 16.12, 17.38, 18.98, 19.51, 22.67, 23.15, 23.36, 23.98, 25.15, 25.32, 25.90, 26.06, 28.82, 29.21, 29.59, 30.94, 31.88, 32.67, 34.00, 35.04, and 38.37, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
D.7 Any of Crystalline Form D or D.1-D.6, wherein the Crystalline Form D exhibits an XRPD pattern comprising at least three, e.g., at least five, e.g., at least ten, e.g., at least fifteen, e.g., at least twenty, e.g., at least twenty-five, d-spacing (Å) values selected from the group consisting of 25.1, 21.6, 20.8, 12.7, 11.0, 10.5, 8.5, 6.8, 6.4, 5.8, 5.6, 5.5, 5.1, 4.7, 4.5, 3.9, 3.8, 3.7, 3.5, 3.4, 3.1, 3.0, 2.9, 2.8, 2.7, 2.6, and 2.3. For instance, any of Crystalline Form D or D.1-D.6, wherein the Crystalline Form D exhibits an XRPD pattern comprising at least three, e.g., at least five, e.g., at least six, e.g., at least ten, e.g., at least fifteen, d-spacing (Å) values selected from the group consisting of 25.1, 21.6, 20.8, 12.7, 11.0, 10.5, 8.5, 6.8, 6.4, 5.8, 5.5, 5.1, 4.5, 3.9, 3.5, 3.4, and 2.3.
D.8 Any of Crystalline Form D or D.1-D.7, wherein the Crystalline Form D exhibits an XRPD pattern comprising d-spacing (Å) values of 25.1, 21.6, 20.8, 12.7, 11.0, 10.5, 8.5, 6.8, 6.4, 5.8, 5.5, 5.1, 4.5, 3.9, 3.5, 3.4, and 2.3. For instance, any of Crystalline Form D or D.1-D.7, wherein the Crystalline Form D exhibits an XRPD pattern comprising d-spacing (Å) values of 25.1, 21.6, 20.8, 12.7, 11.0, 10.5, 8.5, 6.8, 6.4, 5.8, 5.6, 5.5, 5.1, 4.7, 4.5, 3.9, 3.8, 3.7, 3.5, 3.4, 3.1, 3.0, 2.9, 2.8, 2.7, 2.6, and 2.3.
D.9 Any of Crystalline Form D or D.1-D.8, wherein the Crystalline Form D exhibits an XRPD pattern comprising at least three, e.g., at least five, e.g., at least ten, e.g., at least fifteen, e.g., at least twenty, e.g., at least twenty-five, e.g., at least thirty, d-spacing (Å) values selected from the group consisting of 25.11, 21.59, 20.79, 12.69, 10.97, 10.50, 8.49, 6.75, 6.39, 5.77, 5.58, 5.49, 5.10, 4.67, 4.55, 3.92, 3.84, 3.80, 3.71, 3.54, 3.52, 3.44, 3.42, 3.10, 3.06, 3.02, 2.89, 2.81, 2.74, 2.63, 2.56, and 2.34. For instance, any of Crystalline Form D or D.1-D.8, wherein the Crystalline Form D exhibits an XRPD pattern comprising at least three, e.g., at least five, e.g., at least six, e.g., at least ten, e.g., at least fifteen, d-spacing (Å) values selected from the group consisting of 25.11, 21.59, 20.79, 12.69, 10.97, 10.50, 8.49, 6.75, 6.39, 5.77, 5.49, 5.10, 4.55, 3.92, 3.52, 3.44, 3.42, and 2.34.
D.10 Any of Crystalline Form D or D.1-D.9, wherein the Crystalline Form D exhibits an XRPD pattern comprising d-spacing (Å) values of 25.11, 21.59, 20.79, 12.69, 10.97, 10.50, 8.49, 6.75, 6.39, 5.77, 5.49, 5.10, 4.55, 3.92, 3.52, 3.44, 3.42, and 2.34. For instance, any of Crystalline Form D or D.1-D.9, wherein the Crystalline Form D exhibits an XRPD pattern comprising d-spacing (Å) values of 25.11, 21.59, 20.79, 12.69, 10.97, 10.50, 8.49, 6.75, 6.39, 5.77, 5.58, 5.49, 5.10, 4.67, 4.55, 3.92, 3.84, 3.80, 3.71, 3.54, 3.52, 3.44, 3.42, 3.10, 3.06, 3.02, 2.89, 2.81, 2.74, 2.63, 2.56, and 2.34.
D.11 Any of Crystalline Form D or D.1-D.10, wherein the Crystalline Form D exhibits an XRPD pattern comprising at least three, e.g., at least five, e.g., at least ten, e.g., at least fifteen, e.g., at least twenty, e.g., at least twenty-five, e.g., at least thirty, d-spacing (Å) values selected from the group consisting of 25.113, 21.592, 20.794, 12.689, 10.968, 10.498, 8.498, 6.753, 6.393, 5.766, 5.576, 5.494, 5.099, 4.672, 4.547, 3.919, 3.840, 3.804, 3.709, 3.538, 3.515, 3.437, 3.416, 3.096, 3.055, 3017, 2.888, 2.805, 2.738, 2.635, 2.559, and 2.344. For instance, any of Crystalline Form D or D.1-D.10, wherein the Crystalline Form D exhibits an XRPD pattern comprising at least three, e.g., at least five, e.g., at least six, e.g., at least ten, e.g., at least fifteen, d-spacing (Å) values selected from the group consisting of 25.113, 21.592, 20.794, 12.689, 10.968, 10.498, 8.498, 6.753, 6.393, 5.766, 5.494, 5.099, 4.547, 3.919, 3.515, 3.437, 3.416, and 2.344.
D.12 Any of Crystalline Form D or D.1-D.11, wherein the Crystalline Form D exhibits an XRPD pattern comprising d-spacing (Å) values of 25.113, 21.592, 20.794, 12.689, 10.968, 10.498, 8.498, 6.753, 6.393, 5.766, 5.494, 5.099, 4.547, 3.919, 3.515, 3.437, 3.416, and 2.344. For instance, any of Crystalline Form D or D.1-D.11, wherein the Crystalline Form D exhibits an XRPD pattern comprising d-spacing (Å) values of 25.113, 21.592, 20.794, 12.689, 10.968, 10.498, 8.498, 6.753, 6.393, 5.766, 5.576, 5.494, 5.099, 4.672, 4.547, 3.919, 3.840, 3.804, 3.709, 3.538, 3.515, 3.437, 3.416, 3.096, 3.055, 3.017, 2.888, 2.805, 2.738, 2.635, 2.559, and 2.344.
D.13 Any of Crystalline Form D or D.1-D.12, wherein the Crystalline Form D exhibits an XRPD pattern comprising at least three, e.g., at least five, e.g., at least ten, e.g., at least fifteen, e.g., at least twenty, e.g., at least twenty-five, e.g., at least thirty, 2-theta (°) values selected from those set forth in Table D below:

TABLE D
Form D

| Peak Position | Relative | d-spacing |
|---|---|---|
| (°2θ)^{a} | Intensity | value (Å) |
| 3.52 | 44.76 | 25.1134 |
| 4.09 | 33.21 | 21.5918 |
| 4.25 | 100.00 | 20.7938 |
| 6.96 | 1.63 | 12.6889 |
| 8.05 | 5.49 | 10.9680 |
| 8.42 | 36.23 | 10.4975 |
| 10.40 | 3.07 | 8.4984 |
| 13.10 | 1.63 | 6.7532 |
| 13.84 | 1.45 | 6.3926 |
| 15.35 | 8.36 | 5.7661 |
| 15.88 | 8.77 | 5.5757 |
| 16.12 | 25.09 | 5.4936 |
| 17.38 | 39.41 | 5.0986 |
| 18.98 | 1.75 | 4.6720 |
| 19.51 | 4.66 | 4.5472 |
| 22.67 | 18.83 | 3.9191 |
| 23.15 | 2.25 | 3.8398 |
| 23.36 | 2.13 | 3.8042 |
| 23.98 | 5.92 | 3.7087 |
| 25.15 | 13.83 | 3.5375 |
| 25.32 | 5.60 | 3.5152 |
| 25.90 | 17.44 | 3.4373 |
| 26.06 | 19.09 | 3.4160 |
| 28.82 | 2.30 | 3.0955 |
| 29.21 | 2.05 | 3.0549 |
| 29.59 | 2.40 | 3.0167 |
| 30.94 | 14.08 | 2.8881 |
| 31.88 | 1.63 | 2.8050 |
| 32.67 | 1.50 | 2.7384 |
| 34.00 | 6.90 | 2.6347 |
| 35.04 | 2.17 | 2.5588 |
| 38.37 | 9.98 | 2.3441, |

^{a}+/−0.2°2θ per USP guidelines

e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
D.14 Any of Crystalline Form D or D.1-D.13, wherein the Crystalline Form D exhibits an XRPD pattern comprising the 2-theta (°) values set forth in Table D in Crystalline Form D.13, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
D.15 Any of Crystalline Form D or D.1-D.14, wherein the Crystalline Form D exhibits an XRPD pattern comprising at least three, e.g., at least five, e.g., at least ten, e.g., at least fifteen, e.g., at least twenty, e.g., at least twenty-five, e.g., at least thirty, d-spacing (Å) values selected from those set forth in Table D in Crystalline Form D.13.
D.16 Any of Crystalline Form D or D.1-D.15, wherein the Crystalline Form D exhibits an XRPD pattern comprising the d-spacing (Å) values set forth in Table D in Crystalline Form D.13.
D.17 Any of Crystalline Form D or D.1-D.16, wherein the Crystalline Form D exhibits an XRPD pattern comprising at least three, e.g., at least six, e.g., at least ten, e.g., at least fifteen, 2-theta (°) values selected from those set forth in Table DD below:

TABLE DD

| | Relative | d-spacing |
|---|---|---|
| °2θ | Intensity | value (Å) |
| 3.5 | 45 | 25.1134 |
| 4.1 | 33 | 21.5918 |
| 4.3 | 100 | 20.7938 |
| 7.0 | 2 | 12.6889 |
| 8.1 | 5 | 10.9680 |
| 8.4 | 36 | 10.4975 |
| 10.4 | 3 | 8.4984 |
| 13.1 | 2 | 6.7532 |
| 13.8 | 1 | 6.3926 |
| 15.4 | 8 | 5.7661 |
| 16.1 | 25 | 5.4936 |
| 17.4 | 39 | 5.0986 |
| 19.5 | 5 | 4.5472 |
| 22.7 | 19 | 3.9191 |
| 25.3 | 6 | 3.5152 |
| 25.9 | 17 | 3.4373 |
| 26.1 | 19 | 3.4160 |
| 38.4 | 10 | 2.3441, |

e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
D.18 Any of Crystalline Form D or D.1-D.17, wherein the Crystalline Form D exhibits an XRPD pattern comprising the 2-theta (°) values set forth in Table DD in Crystalline Form D.17, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
D.19 Any of Crystalline Form D or D.1-D.18, wherein the Crystalline Form D exhibits an XRPD pattern comprising at least four, e.g., at least six, e.g., at least ten, e.g., at least fifteen, d-spacing (Å) values selected from those set forth in Table DD in Crystalline Form D.17.
D.20 Any of Crystalline Form D or D.1-D.19, wherein the Crystalline Form D exhibits an XRPD pattern comprising the d-spacing (Å) values set forth in Table DD in Crystalline Form D.17.
D.21 Any of Crystalline Form D or D.1-D.20, wherein the Crystalline Form D exhibits an XRPD pattern comprising characteristic 2-theta (°) values selected from any of the 2-theta (°) values set forth in any of Crystalline Form D or D.1-D.20 above, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
D.22 Any of Crystalline Form D or D.1-D.21, wherein the Crystalline Form D exhibits an XRPD pattern comprising representative 2-theta (°) values selected from any of the 2-theta (°) values set forth in any of Crystalline Form D or D.1-D.20 above, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
D.23 Any of Crystalline Form D or D.1-D.22, wherein the Crystalline Form D exhibits an XRPD pattern comprising characteristic d-spacing (Å) values selected from any of the d-spacing (Å) values set forth in any of Crystalline Form D or D.1-D.20 above.
D.24 Any of Crystalline Form D or D.1-D.123, wherein the Crystalline Form D exhibits an XRPD pattern comprising representative d-spacing (Å) values selected from any of the d-spacing (Å) values set forth in any of Crystalline Form D or D.1-D.20 above.
D.25 Any of Crystalline Form D or D.1-D.24, wherein the Crystalline Form D exhibits an XRPD pattern comprising at least three, e.g., at least five, e.g., at least ten, e.g., at least fifteen, e.g., at least twenty, e.g., at least twenty-five, e.g., at least thirty, e.g., all, of the peaks of the XRPD shown in FIG. 18, FIG. 21, and/or FIG. 37, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
D.26 Any of Crystalline Form D or D.1-D.25, wherein the Crystalline Form D exhibits an XRPD pattern comprising the characteristic peaks of the XRPD shown in FIG. 18, FIG. 21, and/or FIG. 37, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
D.27 Any of Crystalline Form D or D.1-D.26, wherein the Crystalline Form D exhibits an XRPD pattern comprising the representative peaks of the XRPD shown in FIG. 18, FIG. 21, and/or FIG. 37, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
D.28 Any of Crystalline Form D or D.1-D.27, wherein the Crystalline Form D exhibits an XRPD corresponding to FIG. 18, FIG. 21, and/or FIG. 37, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
D.29 Any of Crystalline Form D or D.1-D.28, wherein the Crystalline Form D exhibits a differential scanning calorimetry (DSC) thermogram comprising an endotherm between 60-70° C., e.g., between 60-65° C. For instance, any of Crystalline Form D or D.1-D.28, wherein the Crystalline Form D exhibits a differential scanning calorimetry (DSC) thermogram comprising an endotherm at 62° C. (e.g., 62.1° C.).
D.30 Any of Crystalline Form D or D.1-D.29, wherein the Crystalline Form D exhibits a differential scanning calorimetry (DSC) thermogram comprising an endotherm between 120-130° C., e.g., between 120-125° C. For instance, any of Crystalline Form D or D.1-D.29, wherein the Crystalline Form D exhibits a differential scanning calorimetry (DSC) thermogram comprising an endotherm at 122° C. (e.g., 122.5° C.).
D.31 Any of Crystalline Form D or D.1-D.30, wherein the Crystalline Form D exhibits a differential scanning calorimetry (DSC) thermogram as shown in FIG. 19.
D.32 Any of Crystalline Form D or D.1-D.31, wherein the Crystalline Form D exhibits a thermogravimetric analysis (TGA) thermogram comprising weight loss between start (e.g., 25° C.) and 130° C., e.g., between start (e.g., 25° C.) and 125° C., e.g., a weight loss of 9-10 weight %, e.g., a weight loss of 9.8 weight %.
D.33 Any of Crystalline Form D or D.1-D.32, wherein the Crystalline Form D exhibits a thermogravimetric analysis (TGA) thermogram comprising a weight loss corresponding to at least 2 moles of water, e.g., 2.2 moles of water.
D.34 Any of Crystalline Form D or D.1-D.33, wherein the Crystalline Form D exhibits a thermogravimetric analysis (TGA) thermogram as shown in FIG. 20.
D.35 Any of Crystalline Form D or D.1-D.28, wherein the Crystalline Form D is a hydrate. For instance, any of Crystalline Form D or D.1-D.28, wherein the Crystalline Form D comprises at least 2 moles of water, e.g., about 2.2 moles of water.
D.36 Any of Crystalline Form D or D.1-D.35, wherein the Crystalline Form D exhibits an FT-Raman spectrum comprising peaks (cm⁻¹) of 1623, 1537, 1401, 1386, 1343, 1209, and 1167. For instance, any of Crystalline Form D or D.1-D.35, wherein the Crystalline Form D exhibits an FT-Raman spectrum comprising peaks (cm⁻¹) of 2919, 1686, 1623, 1537, 1444, 1401, 1343, 1261, 1209, 1167, 1118, 1019, and 965. For instance, any of Crystalline Form D or D.1-D.35, wherein the Crystalline Form D exhibits Raman peaks (cm⁻¹) of 2919, 1686, 1623, 1537, 1444, 1401, 1386, 1343, 1261, 1209, 1167, 1118, 1019, and 965.
D.37 Any of Crystalline Form D or D.1-D.36, wherein the Crystalline Form D exhibits characteristic Raman peaks (cm⁻¹) selected from those set forth in Crystalline Form D.36.
D.38 Any of Crystalline Form D or D.1-D.37, wherein the Crystalline Form D does not exhibit a melting point based on DSC data.
D.39 Any of Crystalline Form D or D.1-D.38, wherein the Crystalline Form D is made as described in any of Process 1 or 1.1 et seq. vide infra or as described in any of Process 3 or 3.1 et seq. vide infra.
D.40 Any of Crystalline Form D or D.1-D.39, wherein the Crystalline Form D is made as described in any of the examples that produce Crystalline Form D.
D.41 Any of Crystalline Form D or D.1-D.40, wherein any of the listed 2-theta (°) values of an XRPD pattern have an acceptable deviation of ±0.2°.
D.42 Any of Crystalline Form D or D.1-D.41, wherein the 2-theta (°) values of an XRPD pattern have an acceptable deviation of ±0.2°.
D.43 Any of Crystalline Form D or D.1-D.42, wherein the Crystalline Form D is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of the amorphous form.
D.44 Any of Crystalline Form D or D.1-D.43, wherein the Crystalline Form D is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of any other crystalline form.
D.45 Any of Crystalline Form D or D.1-D.44, wherein the Crystalline Form D is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of the amorphous form and any other crystalline form.
D.46 Any of Crystalline Form D or D.1-D.45, wherein the Crystalline Form D is formulated as described in any of Process 2 or 2.1 et seq. vide infra.
D.47 Any of Crystalline Form D, D.1-D.42, or D.46, wherein the Crystalline Form D is in a mixture with another crystalline form and/or amorphous form of trans sodium crocetinate.
D.48 Any of Crystalline Form D or D.1-D.47, wherein the Crystalline Form D is ≥96% w/w pure by HPLC, e.g., ≥97% w/w pure by HPLC, e.g., ≥98% w/w pure by HPLC, e.g., ≥99% w/w pure by HPLC.

Also provided is Crystalline Form E (also called Form E herein) of trans sodium crocetinate (Formula I above). Further provided is Crystalline Form E as follows:
E.1 Crystalline Form E, wherein the Crystalline Form E exhibits an XRPD pattern comprising 2-theta (°) values 4.2, 8.4, 16.0, and 17.6, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. Or, for instance, Crystalline Form E, wherein the Crystalline Form E exhibits an XRPD pattern comprising d-spacing (Å) values of 20.9, 10.5, 5.5, and 5.0.
E.2 Crystalline Form E or E.1, wherein the Crystalline Form E exhibits an XRPD pattern comprising 2-theta (°) values of 4.2, 8.4, 16.0, 17.6, 18.5, and 19.8, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. Or, for instance, Crystalline Form E or E.1, wherein the Crystalline Form E exhibits an XRPD pattern comprising d-spacing (Å) values of 20.9, 10.5, 5.5, 5.0, 4.8, and 4.5.
E.3 Any of Crystalline Form E, E.1, or E.2, wherein the Crystalline Form E exhibits an XRPD pattern comprising at least three, e.g., at least four, e.g., at least five, e.g., at least six, e.g., at least ten, e.g., at least fifteen, e.g., at least twenty, 2-theta (°) values selected from the group consisting of 4.2, 8.4, 16.0, 16.8, 17.6, 18.5, 19.8, 21.0, 22.5, 23.6, 23.9, 24.3, 25.3, 27.2, 28.6, 29.6, 30.4, 30.8, 31.3, 33.9, 34.3, 35.7, and 38.3, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. For instance, any of Crystalline Form E, E.1, or E.2, wherein the Crystalline Form E exhibits an XRPD pattern comprising at least three, e.g., at least four, e.g., at least five, e.g., at least six, e.g., at least ten, e.g., at least fifteen, 2-theta (°) values selected from the group consisting of 4.2, 8.4, 16.0, 16.8, 17.6, 18.5, 19.8, 21.0, 23.6, 23.9, 24.3, 25.3, 27.2, 28.6, 30.8, 31.3, 33.9, and 38.3, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
E.4 Any of Crystalline Form E or E.1-E.3, wherein the Crystalline Form E exhibits an XRPD pattern comprising 2-theta (°) values of 4.2, 8.4, 16.0, 16.8, 17.6, 18.5, 19.8, 21.0, 23.6, 23.9, 24.3, 25.3, 27.2, 28.6, 30.8, 31.3, 33.9, and 38.3, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. For instance, any of Crystalline Form E or E.1-E.3, wherein the Crystalline Form E exhibits an XRPD pattern comprising 2-theta (°) values of 4.2, 8.4, 16.0, 16.8, 17.6, 18.5, 19.8, 21.0, 22.5, 23.6, 23.9, 24.3, 25.3, 27.2, 28.6, 29.6, 30.4, 30.8, 31.3, 33.9, 34.3, 35.7, and 38.3, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
E.5 Any of Crystalline Form E or E.1-E.4, wherein the Crystalline Form E exhibits an XRPD pattern comprising at least three, e.g., at least four, e.g., at least five, e.g., at least six, e.g., at least ten, e.g., at least fifteen, e.g., at least twenty, 2-theta (°) values selected from the group consisting of 4.23, 8.41, 16.00, 16.77, 17.64, 18.48, 19.80, 20.96, 22.50, 23.60, 23.88, 24.31, 25.26, 27.24, 28.64, 29.56, 30.41, 30.81, 31.34, 33.88, 34.34, 35.68, and 38.28, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
E.6 Any of Crystalline Form E or E.1-E.5, wherein the Crystalline Form E exhibits an XRPD pattern comprising 2-theta (°) values of 4.23, 8.41, 16.00, 16.77, 17.64, 18.48, 19.80, 20.96, 22.50, 23.60, 23.88, 24.31, 25.26, 27.24, 28.64, 29.56, 30.41, 30.81, 31.34, 33.88, 34.34, 35.68, and 38.28, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
E.7 Any of Crystalline Form E or E.1-E.6, wherein the Crystalline Form E exhibits an XRPD pattern comprising at least three, e.g., at least four, e.g., at least five, e.g., at least six e.g., at least ten, e.g., at least fifteen, d-spacing (Å) values selected from the group consisting of 20.9, 10.5, 5.5, 5.3, 5.0, 4.8, 4.5, 4.2, 3.9, 3.8, 3.7, 3.5, 3.3, 3.1, 3.0, 2.9, 2.6, 2.5, and 2.3. For instance, any of Crystalline Form E or
E.1-E.6, wherein the Crystalline Form E exhibits an XRPD pattern comprising at least three, e.g., at least four, e.g., at least five, e.g., at least six e.g., at least ten, e.g., at least fifteen, d-spacing (Å) values selected from the group consisting of 20.9, 10.5, 5.5, 5.3, 5.0, 4.8, 4.5, 4.2, 3.8, 3.7, 3.5, 3.3, 3.1, 2.9, 2.6, and 2.3.
E.8 Any of Crystalline Form E or E.1-E.7, wherein the Crystalline Form E exhibits an XRPD pattern comprising d-spacing (Å) values of 20.9, 10.5, 5.5, 5.3, 5.0, 4.8, 4.5, 4.2, 3.8, 3.7, 3.5, 3.3, 3.1, 2.9, 2.6, and 2.3. For instance, any of Crystalline Form E or E.1-E.7, wherein the Crystalline Form E exhibits an XRPD pattern comprising d-spacing (Å) values of 20.9, 10.5, 5.5, 5.3, 5.0, 4.8, 4.5, 4.2, 3.9, 3.8, 3.7, 3.5, 3.3, 3.1, 3.0, 2.9, 2.6, 2.5, and 2.3.
E.9 Any of Crystalline Form E or E.1-E.8, wherein the Crystalline Form E exhibits an XRPD pattern comprising at least three, e.g., at least four, e.g., at least five, e.g., at least six, e.g., at least ten, e.g., at least fifteen, e.g., at least twenty, d-spacing (Å) values selected from the group consisting of 20.86, 10.51, 5.54, 5.28, 5.02, 4.80, 4.48, 4.23, 3.95, 3.77, 3.72, 3.66, 3.53, 3.27, 3.11, 3.02, 2.94, 2.90, 2.85, 2.64, 2.61, 2.51, and 2.35. For instance, any of Crystalline Form E or E.1-E.8, wherein the Crystalline Form E exhibits an XRPD pattern comprising at least three, e.g., at least four, e.g., at least five, e.g., at least six e.g., at least ten, e.g., at least fifteen, d-spacing (Å) values selected from the group consisting of 20.86, 10.51, 5.54, 5.28, 5.02, 4.80, 4.48, 4.23, 3.77, 3.72, 3.66, 3.52, 3.28, 3.11, 2.90, 2.85, 2.64, and 2.35.
E.10 Any of Crystalline Form E or E.1-E.9, wherein the Crystalline Form E exhibits an XRPD pattern comprising d-spacing (Å) values of 20.86, 10.51, 5.54, 5.28, 5.02, 4.80, 4.48, 4.23, 3.77, 3.72, 3.66, 3.52, 3.28, 3.11, 2.90, 2.85, 2.64, and 2.35. For instance, any of Crystalline Form E or E.1-E.9, wherein the Crystalline Form E exhibits an XRPD pattern comprising d-spacing (Å) values of 20.86, 10.51, 5.54, 5.28, 5.02, 4.80, 4.48, 4.23, 3.95, 3.77, 3.72, 3.66, 3.53, 3.27, 3.11, 3.02, 2.94, 2.90, 2.85, 2.64, 2.61, 2.51, and 2.35.
E.11 Any of Crystalline Form E or E.1-E.10, wherein the Crystalline Form E exhibits an XRPD pattern comprising at least three, e.g., at least four, e.g., at least five, e.g., at least six, e.g., at least ten, e.g., at least fifteen, e.g., at least twenty, d-spacing (Å) values selected from the group consisting of 20.857, 10.507, 5.536, 5.281, 5.024, 4.797, 4.481, 4.234, 3.948, 3.766, 3.723, 3.658, 3.523, 3.271, 3.114, 3.020, 2.937, 2.900, 2.852, 2.644, 2.609, 2.514, and 2.350. For instance, any of Crystalline Form E or E.1-E.10, wherein the Crystalline Form E exhibits an XRPD pattern comprising at least three, e.g., at least four, e.g., at least five, e.g., at least six e.g., at least ten, e.g., at least fifteen, d-spacing (Å) values selected from the group consisting of 20.857, 10.507, 5.536, 5.281, 5.024, 4.797, 4.481, 4.234, 3.766, 3.723, 3.658, 3.523, 3.271, 3.114, 2.900, 2.852, 2.644, and 2.350.
E.12 Any of Crystalline Form E or E.1-E.11, wherein the Crystalline Form E exhibits an XRPD pattern comprising d-spacing (Å) values of 20.857, 10.507, 5.536, 5.281, 5.024, 4.797, 4.481, 4.234, 3.766, 3.723, 3.658, 3.523, 3.271, 3.114, 2.900, 2.852, 2.644, and 2.350. For instance, any of Crystalline Form E or E.1-E.11, wherein the Crystalline Form E exhibits an XRPD pattern comprising d-spacing (Å) values of 20.857, 10.507, 5.536, 5.281, 5.024, 4.797, 4.481, 4.234, 3.948, 3.766, 3.723, 3.658, 3.523, 3.271, 3.114, 3.020, 2.937, 2.900, 2.852, 2.644, 2.609, 2.514, and 2.350.
E.13 Any of Crystalline Form E or E.1-E.12, wherein the Crystalline Form E exhibits an XRPD pattern comprising at least three, e.g., at least four, e.g., at least five, e.g., at least six, e.g., at least ten, e.g., at least fifteen, e.g., at least twenty, e.g., at least twenty-five, e.g., at least thirty, 2-theta (°) values selected from those set forth in Table E below:

TABLE E
Form E

| Peak Position | Relative | d-spacing |
|---|---|---|
| (°2θ)^{a} | Intensity | value (Å) |
| 4.23 | 100.00 | 20.8569 |
| 8.41 | 30.55 | 10.5071 |
| 16.00 | 16.55 | 5.5363 |
| 16.77 | 1.95 | 5.2810 |
| 17.64 | 63.37 | 5.0242 |
| 18.48 | 3.39 | 4.7973 |
| 19.80 | 10.12 | 4.4806 |
| 20.96 | 2.46 | 4.2344 |
| 22.50 | 2.93 | 3.9477 |
| 23.60 | 47.72 | 3.7664 |
| 23.88 | 14.20 | 3.7230 |
| 24.31 | 43.81 | 3.6584 |
| 25.26 | 1.55 | 3.5228 |
| 27.24 | 4.77 | 3.2711 |
| 28.64 | 15.37 | 3.1140 |
| 29.56 | 1.10 | 3.0196 |
| 30.41 | 9.92 | 2.9368 |
| 30.81 | 5.02 | 2.8997 |
| 31.34 | 5.61 | 2.8524 |
| 33.88 | 6.61 | 2.6438 |
| 34.34 | 10.18 | 2.6094 |
| 35.68 | 4.73 | 2.5141 |
| 38.28 | 6.62 | 2.3496, |

^{a}+/−0.2°2θ per USP guidelines

e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
E.14 Any of Crystalline Form E or E.1-E.13, wherein the Crystalline Form E exhibits an XRPD pattern comprising the 2-theta (°) values set forth in Table E in Crystalline Form E.13, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
E.15 Any of Crystalline Form E or E.1-E.14, wherein the Crystalline Form E exhibits an XRPD pattern comprising at least three, e.g., at least four, e.g., at least five, e.g., at least six, e.g., at least ten, e.g., at least fifteen, e.g., at least twenty, e.g., at least twenty-five, e.g., at least thirty, d-spacing (Å) values selected from those set forth in Table E in Crystalline Form E.13.
E.16 Any of Crystalline Form E or E.1-E.15, wherein the Crystalline Form E exhibits an XRPD pattern comprising the d-spacing (Å) values set forth in Table E in Crystalline Form E.13.
E.17 Any of Crystalline Form E or E.1-E.16, wherein the Crystalline Form E exhibits an XRPD pattern comprising at least three, e.g., at least four, e.g., at least five, e.g., at least six, e.g., at least ten, e.g., at least fifteen, 2-theta (°) values selected from those set forth in Table EE below:

TABLE EE

| °2θ | Relative Intensity | d-spacing value (Å) |
|---|---|---|
| 4.2 | 100 | 20.8569 |
| 8.4 | 31 | 10.5071 |
| 16.0 | 17 | 5.5363 |
| 16.8 | 2 | 5.2810 |
| 17.6 | 63 | 5.0242 |
| 18.5 | 3 | 4.7973 |
| 19.8 | 10 | 4.4806 |
| 21.0 | 2 | 4.2344 |
| 23.6 | 48 | 3.7664 |
| 23.9 | 14 | 3.7230 |
| 24.3 | 44 | 3.6584 |
| 25.3 | 2 | 3.5228 |
| 27.2 | 5 | 3.2711 |
| 28.6 | 15 | 3.1140 |
| 30.8 | 5 | 2.8997 |
| 31.3 | 6 | 2.8524 |
| 33.9 | 7 | 2.6438 |
| 38.3 | 7 | 2.3496, |

e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
E.18 Any of Crystalline Form E or E.1-E.17, wherein the Crystalline Form E exhibits an XRPD pattern comprising the 2-theta (°) values set forth in Table EE in Crystalline Form E.17, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
E.19 Any of Crystalline Form E or E.1-E.18, wherein the Crystalline Form E exhibits an XRPD pattern comprising at least three, e.g., at least four, e.g., at least five, e.g., at least six, e.g., at least ten, e.g., at least fifteen, d-spacing (Å) values selected from those set forth in Table EE in Crystalline Form E.17.
E.20 Any of Crystalline Form E or E.1-E.19, wherein the Crystalline Form E exhibits an XRPD pattern comprising the d-spacing (Å) values set forth in Table EE in Crystalline Form E.17.
E.21 Any of Crystalline Form E or E.1-E.20, wherein the Crystalline Form E exhibits an XRPD pattern comprising characteristic 2-theta (°) values selected from any of the 2-theta (°) values set forth in any of Crystalline Form E or E.1-E.20 above, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
E.22 Any of Crystalline Form E or E.1-E.21, wherein the Crystalline Form E exhibits an XRPD pattern comprising representative 2-theta (°) values selected from any of the 2-theta (°) values set forth in any of Crystalline Form E or E.1-E.20 above, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
E.23 Any of Crystalline Form E or E.1-E.22, wherein the Crystalline Form E exhibits an XRPD pattern comprising characteristic d-spacing (Å) values selected from any of the d-spacing (Å) values set forth in any of Crystalline Form E or E.1-E.20 above.
E.24 Any of Crystalline Form E or E.1-E.23, wherein the Crystalline Form E exhibits an XRPD pattern comprising representative d-spacing (Å) values selected from any of the d-spacing (Å) values set forth in any of Crystalline Form E or E.1-E.20 above.
E.25 Any of Crystalline Form E or E.1-E.24, wherein the Crystalline Form E exhibits an XRPD pattern comprising at least three, e.g., at least five, e.g., at least ten, e.g., at least fifteen, e.g., at least twenty, e.g., all, of the peaks of the XRPD shown in FIG. 22 and/or FIG. 49, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
E.26 Any of Crystalline Form E or E.1-E.25, wherein the Crystalline Form E exhibits an XRPD pattern comprising the characteristic peaks of the XRPD shown in FIG. 22 and/or FIG. 49, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
E.27 Any of Crystalline Form E or E.1-E.26, wherein the Crystalline Form E exhibits an XRPD pattern comprising the representative peaks of the XRPD shown in FIG. 22 and/or FIG. 49, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
E.28 Any of Crystalline Form E or E.1-E.27, wherein the Crystalline Form E exhibits an XRPD corresponding to FIG. 22 and/or FIG. 49, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
E.29 Any of Crystalline Form E or E.1-E.28, wherein the Crystalline Form E exhibits a differential scanning calorimetry (DSC) thermogram comprising an endotherm between 60-70° C., e.g., between 60-65° C. For instance, any of Crystalline Form E or E.1-E.22, wherein the Crystalline Form E exhibits a differential scanning calorimetry (DSC) thermogram comprising an endotherm at 62° C. (e.g., 61.6° C.).
E.30 Any of Crystalline Form E or E.1-E.29, wherein the Crystalline Form E exhibits a differential scanning calorimetry (DSC) thermogram as shown in FIG. 23.
E.31 Any of Crystalline Form E or E.1-E.30, wherein the Crystalline Form E exhibits a thermogravimetric analysis (TGA) thermogram comprising weight loss between start (e.g., 25° C.) and 80° C., e.g., between start (e.g., 25° C.) and 70° C., e.g., a weight loss of 2-3 weight %, e.g., a weight loss of 2.2 weight %.
E.32 Any of Crystalline Form E or E.1-E.31, wherein the Crystalline Form E exhibits a thermogravimetric analysis (TGA) thermogram comprising a weight loss corresponding to 0.5 moles of water.
E.33 Any of Crystalline Form E or E.1-E.32, wherein the Crystalline Form E exhibits a thermogravimetric analysis (TGA) thermogram as shown in FIG. 24.
E.34 Any of Crystalline Form E or E.1-E.33, wherein the Crystalline Form E is a hydrate. For instance, any of Crystalline Form E or E.1-E.33, wherein the Crystalline Form E comprises about 0.5 moles of water (e.g., hemi-hydrate).
E.35 Any of Crystalline Form E or E.1-E.34, wherein the Crystalline Form E exhibits an FT-Raman spectrum comprising peaks (cm⁻¹) of 1621, 1537, 1414, 1331, 1266, 1202, 1159, and 1018. For instance, any of Crystalline Form E or E.1-E.34, wherein the Crystalline Form E exhibits an FT-Raman spectrum comprising peaks (cm⁻¹) of 2916, 1621, 1537, 1414, 1331, 1310, 1266, 1202, 1159, 1018, 980, 890, and 845.
E.36 Any of Crystalline Form E or E.1-E.35, wherein the Crystalline Form E exhibits characteristic Raman peaks (cm⁻¹) selected from those set forth in Crystalline Form E.35.
E.37 Any of Crystalline Form E or E.1-E.36, wherein the Crystalline Form E does not exhibit a melting point based on DSC data.
E.38 Any of Crystalline Form E or E.1-E.37, wherein the Crystalline Form E is made as described in any of Process 1 or 1.1 et seq. vide infra or as described in any of Process 3 or 3.1 et seq. vide infra.
E.39 Any of Crystalline Form E or E.1-E.38, wherein the Crystalline Form E is made as described in any of the examples that produce Crystalline Form E.
E.40 Any of Crystalline Form E or E.1-E.39, wherein any of the listed 2-theta (°) values of an XRPD pattern have an acceptable deviation of ±0.2°.
E.41 Any of Crystalline Form E or E.1-E.40, wherein the 2-theta (°) values of an XRPD pattern have an acceptable deviation of ±0.2°.
E.42 Any of Crystalline Form E or E.1-E.41, wherein the Crystalline Form E is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of the amorphous form.
E.43 Any of Crystalline Form E or E.1-E.42, wherein the Crystalline Form E is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of any other crystalline form.
E.44 Any of Crystalline Form E or E.1-E.43, wherein the Crystalline Form E is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of the amorphous form and any other crystalline form.
E.45 Any of Crystalline Form E or E.1-E.44, wherein the Crystalline Form E is formulated as described in any of Process 2 or 2.1 et seq. vide infra.
E.46 Any of Crystalline Form E, E.1-E.41, or E.45, wherein the Crystalline Form E is in a mixture with another crystalline form and/or amorphous form of trans sodium crocetinate.
E.47 Any of Crystalline Form E or E.1-E.47, wherein the Crystalline Form E is ≥96% w/w pure by HPLC, e.g., ≥97% w/w pure by HPLC, e.g., ≥98% w/w pure by HPLC, e.g., ≥99% w/w pure by HPLC.

Also provided is Crystalline Form F (also called Form F herein) of trans sodium crocetinate (Formula I above). Further provided is Crystalline Form F as follows:
F.1 Crystalline Form F, wherein the Crystalline Form F exhibits an XRPD pattern comprising 2-theta (°) values 4.2, 8.4, 16.8, and 17.5, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. Or, for instance, Crystalline Form F, wherein the Crystalline Form F exhibits an XRPD pattern comprising d-spacing (Å) values of 20.9, 10.5, 5.3, and 5.1.
F.2 Crystalline Form F or F.1, wherein the Crystalline Form F exhibits an XRPD pattern comprising 2-theta (°) values of 4.2, 8.4, 12.6, 16.8, 17.5, and 21.1, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. Or, for instance, Crystalline Form F or F.1, wherein the Crystalline Form F exhibits an XRPD pattern comprising d-spacing (Å) values of 20.9, 10.5, 7.0, 5.3, 5.1, and 4.2.
F.3 Any of Crystalline Form F, F.1, or F.2, wherein the Crystalline Form F exhibits an XRPD pattern comprising at least three, e.g., at least four, e.g., at least five, e.g., at least six, e.g., at least ten, 2-theta (°) values selected from the group consisting of 4.2, 8.4, 12.6, 16.8, 17.5, 21.1, 23.9, 24.4, 25.4, 29.7, 30.8, 34.0, and 38.4, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
F.4 Any of Crystalline Form F or F.1-F.3, wherein the Crystalline Form F exhibits an XRPD pattern comprising 2-theta (°) values of 4.2, 8.4, 12.6, 16.8, 17.5, 21.1, 23.9, 24.4, 25.4, 29.7, 30.8, 34.0, and 38.4, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
F.5 Any of Crystalline Form F or F.1-F.4, wherein the Crystalline Form F exhibits an XRPD pattern comprising at least three, e.g., at least four, e.g., at least five, e.g., at least six, e.g., at least ten, 2-theta (°) values selected from the group consisting of 4.23, 8.40, 12.64, 16.84, 17.48, 21.05, 23.86, 24.40, 25.37, 29.66, 30.82, 34.02, and 38.43, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
F.6 Any of Crystalline Form F or F.1-F.5, wherein the Crystalline Form F exhibits an XRPD pattern comprising 2-theta (°) values of 4.23, 8.40, 12.64, 16.84, 17.48, 21.05, 23.86, 24.40, 25.37, 29.66, 30.82, 34.02, and 38.43, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
F.7 Any of Crystalline Form F or F.1-F.6, wherein the Crystalline Form F exhibits an XRPD pattern comprising at least three, e.g., at least four, e.g., at least five, e.g., at least six, e.g., at least ten, d-spacing (Å) values selected from the group consisting of 20.9, 10.5, 7.0, 5.3, 5.1, 4.2, 3.7, 3.6, 3.5, 3.0, 2.9, 2.6, and 2.3.
F.8 Any of Crystalline Form F or F.1-F.7, wherein the Crystalline Form F exhibits an XRPD pattern comprising d-spacing (Å) values of 20.9, 10.5, 7.0, 5.3, 5.1, 4.2, 3.7, 3.6, 3.5, 3.0, 2.9, 2.6, and 2.3.
F.9 Any of Crystalline Form F or F.1-F.8, wherein the Crystalline Form F exhibits an XRPD pattern comprising at least three, e.g., at least four, e.g., at least five, e.g., at least six, e.g., at least ten, d-spacing (Å) values selected from the group consisting of 20.90, 10.52, 7.00, 5.26, 5.07, 4.22, 3.73, 3.65, 3.51, 3.01, 2.90, 2.63, and 2.34.
F.10 Any of Crystalline Form F or F.1-F.9, wherein the Crystalline Form F exhibits an XRPD pattern comprising d-spacing (Å) values of 20.90, 10.52, 7.00, 5.26, 5.07, 4.22, 3.73, 3.65, 3.51, 3.01, 2.90, 2.63, and 2.34.
F.11 Any of Crystalline Form F or F.1-F.10, wherein the Crystalline Form F exhibits an XRPD pattern comprising at least three, e.g., at least four, e.g., at least five, e.g., at least six, e.g., at least ten, d-spacing (Å) values selected from the group consisting of 20.897, 10.519, 6.999, 5.259, 5.071, 4.216, 3.726, 3.646, 3.508, 3.010, 2.899, 2.633, and 2.341.
F.12 Any of Crystalline Form F or F.1-F.11, wherein the Crystalline Form F exhibits an XRPD pattern comprising d-spacing (Å) values of 20.897, 10.519, 6.999, 5.259, 5.071, 4.216, 3.726, 3.646, 3.508, 3.010, 2.899, 2.633, and 2.341.
F.13 Any of Crystalline Form F or F.1-F.12, wherein the Crystalline Form F exhibits an XRPD pattern comprising at least three, e.g., at least four, e.g., at least five, e.g., at least six, e.g., at least ten, 2-theta (°) values selected from those set forth in Table F below:

TABLE F
Form F

| Peak Position (°2θ)^{a} | Relative Intensity | d-spacing value (Å) |
|---|---|---|
| 4.23 | 100.00 | 20.8970 |
| 8.40 | 50.98 | 10.5192 |
| 12.64 | 1.86 | 6.9994 |
| 16.84 | 2.23 | 5.2591 |
| 17.48 | 4.31 | 5.0707 |
| 21.05 | 2.95 | 4.2164 |
| 23.86 | 1.68 | 3.7259 |
| 24.40 | 4.20 | 3.6456 |
| 25.37 | 3.32 | 3.5076 |
| 29.66 | 3.05 | 3.0096 |
| 30.82 | 2.25 | 2.8993 |
| 34.02 | 7.68 | 2.6329 |
| 38.43 | 14.13 | 2.3405, |

^{a}+/−0.2 °2θ per USP guidelines

e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
F.14 Any of Crystalline Form F or F.1-F.13, wherein the Crystalline Form F exhibits an XRPD pattern comprising the 2-theta (°) values set forth in Table F in Crystalline Form F.13, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
F.15 Any of Crystalline Form F or F.1-F.14, wherein the Crystalline Form F exhibits an XRPD pattern comprising at least three, e.g., at least four, e.g., at least five, e.g., at least six, e.g., at least ten, d-spacing (Å) values selected from those set forth in Table F in Crystalline Form F.13.
F.16 Any of Crystalline Form F or F.1-F.15, wherein the Crystalline Form F exhibits an XRPD pattern comprising the d-spacing (Å) values set forth in Table F in Crystalline Form F.13.
F.17 Any of Crystalline Form F or F.1-F.16, wherein the Crystalline Form F exhibits an XRPD pattern comprising at least four, e.g., at least six, e.g., at least ten, 2-theta (°) values selected from those set forth in Table FF below:

TABLE FF

| °2θ | Relative Intensity | d-spacing value (Å) |
|---|---|---|
| 4.2 | 100 | 20.8970 |
| 8.4 | 51 | 10.5192 |
| 12.6 | 2 | 6.9994 |
| 16.8 | 2 | 5.2591 |
| 17.5 | 4 | 5.0707 |
| 21.1 | 3 | 4.2164 |
| 23.9 | 2 | 3.7259 |
| 24.4 | 4 | 3.6456 |
| 25.4 | 3 | 3.5076 |
| 29.7 | 3 | 3.0096 |
| 30.8 | 2 | 2.8993 |
| 34.0 | 8 | 2.6329 |
| 38.4 | 14 | 2.3405, |

e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
F.18 Any of Crystalline Form F or F.1-F.17, wherein the Crystalline Form F exhibits an XRPD pattern comprising the 2-theta (°) values set forth in Table FF in Crystalline Form F.17, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
F.19 Any of Crystalline Form F or F.1-F.18, wherein the Crystalline Form F exhibits an XRPD pattern comprising at least four, e.g., at least six, e.g., at least ten, d-spacing (Å) values selected from those set forth in Table FF in Crystalline Form F.17.
F.20 Any of Crystalline Form F or F.1-F.19, wherein the Crystalline Form F exhibits an XRPD pattern comprising the d-spacing (Å) values set forth in Table FF in Crystalline Form F.17.
F.21 Any of Crystalline Form F or F.1-F.20, wherein the Crystalline Form F exhibits an XRPD pattern comprising characteristic 2-theta (°) values selected from any of the 2-theta (°) values set forth in any of Crystalline Form F or F.1-F.20 above, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
F.22 Any of Crystalline Form F or F.1-F.21, wherein the Crystalline Form F exhibits an XRPD pattern comprising representative 2-theta (°) values selected from any of the 2-theta (°) values set forth in any of Crystalline Form F or F.1-F.20 above, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
F.23 Any of Crystalline Form F or F.1-F.22, wherein the Crystalline Form F exhibits an XRPD pattern comprising characteristic d-spacing (Å) values selected from any of the d-spacing (Å) values set forth in any of Crystalline Form F or F.1-F.20 above.
F.24 Any of Crystalline Form F or F.1-F.23, wherein the Crystalline Form F exhibits an XRPD pattern comprising representative d-spacing (Å) values selected from any of the d-spacing (Å) values set forth in any of Crystalline Form F or F.1-F.20 above.
F.25 Any of Crystalline Form F or F.1-F.24, wherein the Crystalline Form F exhibits an XRPD pattern comprising at least three, e.g., at least five, e.g., at least ten, e.g., all, of the peaks of the XRPD shown in FIG. 25, FIG. 27, and/or FIG. 36, e.g., XRPD shown in FIG. 25 and/or FIG. 27, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. FIG. 26 the same XRPD as FIG. 25, but with a different y-axis scale.
F.26 Any of Crystalline Form F or F.1-F.25, wherein the Crystalline Form F exhibits an XRPD pattern comprising the characteristic peaks of the XRPD shown in FIG. 25, FIG. 27, and/or FIG. 36, e.g., XRPD shown in FIG. 25 and/or FIG. 27, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
F.27 Any of Crystalline Form F or F.1-F.26, wherein the Crystalline Form F exhibits an XRPD pattern comprising the representative peaks of the XRPD shown in FIG. 25, FIG. 27, and/or FIG. 36, e.g., XRPD shown in FIG. 25 and/or FIG. 27, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
F.28 Any of Crystalline Form F or F.1-F.27, wherein the Crystalline Form F exhibits an XRPD corresponding to FIG. 25, FIG. 27, and/or FIG. 36, e.g., XRPD shown in FIG. 25 and/or FIG. 27, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
F.29 Any of Crystalline Form F or F.1-F.28, wherein the Crystalline Form F exhibits a differential scanning calorimetry (DSC) thermogram comprising an endotherm between 50-60° C., e.g., between 50-55° C. For instance, any of Crystalline Form F or F.1-F.28, wherein the Crystalline Form F exhibits a differential scanning calorimetry (DSC) thermogram comprising an endotherm at 51° C. (e.g., 51.2° C.).
F.30 Any of Crystalline Form F or F.1-F.29, wherein the Crystalline Form F exhibits a differential scanning calorimetry (DSC) thermogram comprising an endotherm between 90-100° C., e.g., between 90-95° C. For instance, any of Crystalline Form F or F.1-F.29, wherein the Crystalline Form F exhibits a differential scanning calorimetry (DSC) thermogram comprising an endotherm at 93° C. (e.g., 93.1° C.).
F.31 Any of Crystalline Form F or F.1-F.30, wherein the Crystalline Form F exhibits a differential scanning calorimetry (DSC) thermogram as shown in FIG. 28.
F.32 Any of Crystalline Form F or F.1-F.31, wherein the Crystalline Form F exhibits a thermogravimetric analysis (TGA) thermogram comprising weight loss between start (e.g., 25° C.) and 110° C., e.g., between start (e.g., 25° C.) and 100° C., e.g., a weight loss of 0-1 weight %, e.g., a weight loss of 0.5-1 weight %, e.g., a weight loss of 0.7 weight %.
F.33 Any of Crystalline Form F or F.1-F.32, wherein the Crystalline Form F exhibits a thermogravimetric analysis (TGA) thermogram comprising a weight loss corresponding to 0.1 moles of water.
F.34 Any of Crystalline Form F or F.1-F.33, wherein the Crystalline Form F exhibits a thermogravimetric analysis (TGA) thermogram as shown in FIG. 29.
F.35 Any of Crystalline Form F or F.1-F.34, wherein the Crystalline Form F comprises 0.1 moles of water.
F.36 Any of Crystalline Form F or F.1-F.35, wherein the Crystalline Form F exhibits a dynamic (water) vapor sorption (DVS) isotherm comprising 20% or less weight gain upon increasing the relative humidity from 5% to 95%, e.g., 16% or less weight gain upon increasing the relative humidity from 5% to 95%.
F.37 Any of Crystalline Form F or F.1-F.36, wherein the Crystalline Form F exhibits a dynamic (water) vapor sorption (DVS) isotherm as shown in FIG. 43. For instance, any of Crystalline Form F or F.1-F.36, wherein the Crystalline Form F remains unchanged by XRPD after a dynamic (water) vapor sorption (DVS) experiment.
F.38 Any of Crystalline Form F or F.1-F.37, wherein the Crystalline Form F exhibits an FT-Raman spectrum comprising peaks (cm⁻¹) of 1621, 1537, 1414, 1331, 1310, 1266, 1202, 1159, and 1018. Any of Crystalline Form F or F.1-F.37, wherein the Crystalline Form F exhibits an FT-Raman spectrum comprising peaks (cm⁻¹) of 2916, 1621, 1537, 1414, 1331, 1310, 1266, 1202, 1159, 1114, 1018, 981, 891, 845, and 826.
F.39 Any of Crystalline Form F or F.1-F.38, wherein the Crystalline Form F exhibits characteristic Raman peaks (cm⁻¹) selected from those set forth in Crystalline Form F.38.
F.40 Any of Crystalline Form F or F.1-F.39, wherein the Crystalline Form F exhibits does not exhibit a broad endotherm in a differential scanning calorimetry (DSC) thermogram. For instance, any of Crystalline Form F, wherein the Crystalline Form F exhibits does not exhibit a broad endotherm in a differential scanning calorimetry (DSC) thermogram above 100° C.
F.41 Any of Crystalline Form F or F.1-F.40, wherein the Crystalline Form F exhibits does not exhibit an endotherm in a differential scanning calorimetry (DSC) thermogram above 100° C.
F.42 Any of Crystalline Form F or F.1-F.41, wherein the Crystalline Form F exhibits a melting point about 93° C. in a differential scanning calorimetry (DSC) thermogram.
F.43 Any of Crystalline Form F or F.1-F.42, wherein the Crystalline Form F is made as described in any of Process 1 or 1.1 et seq. vide infra or as described in any of Process 3 or 3.1 et seq. vide infra.
F.44 Any of Crystalline Form F or F.1-F.43, wherein the Crystalline Form F is made as described in any of the examples that produce Crystalline Form F.
F.45 Any of Crystalline Form F or F.1-F.44, wherein any of the listed 2-theta (°) values of an XRPD pattern have an acceptable deviation of +0.2°.
F.46 Any of Crystalline Form F or F.1-F.45, wherein the 2-theta (°) values of an XRPD pattern have an acceptable deviation of ±0.2°.
F.47 Any of Crystalline Form F or F.1-F.46, wherein the Crystalline Form F is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of the amorphous form.
F.48 Any of Crystalline Form F or F.1-F.47, wherein the Crystalline Form F is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of any other crystalline form.
F.49 Any of Crystalline Form F or F.1-F.48, wherein the Crystalline Form F is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of the amorphous form and any other crystalline form.
F.50 Any of Crystalline Form F or F.1-F.49, wherein the Crystalline Form F is formulated as described in any of Process 2 or 2.1 et seq. vide infra.
F.51 Any of Crystalline Form F or F.1-F.50, wherein Crystalline Form F is unsolvated (including unhydrated). Any of Crystalline Form F or F.1-F.50, wherein Crystalline Form F is a non-solvate non-hydrate.
F.52 Any of Crystalline Form F, F.1-F.46, F.50, or F.51, wherein the Crystalline Form F is in a mixture with another crystalline form and/or amorphous form of trans sodium crocetinate. For instance, any of Crystalline Form F, F.1-F.46, F.50, or F.51, wherein the Crystalline Form F is in a mixture with Crystalline Form B or B.1 et seq. vide supra. Or, for instance, any of Crystalline Form F, F.1-F.46, F.50, or F.51, wherein the Crystalline Form F is in a mixture with Crystalline Form A or A.1 et seq. vide supra.
F.53 Any of Crystalline Form F or F.1-F.52, wherein the Crystalline Form F remains unchanged after DVS analysis where the sample is exposed up to 95% relative humidity (RH) and down to 5% RH and/or remains unchanged for up to 4 weeks at 25° C./60% relative humidity (RH) and/or for up to 4 weeks at 40° C./75% RH.
F.54 Any of Crystalline Form F or F.1-F.53, wherein the Crystalline Form F is ≥96% w/w pure by HPLC, e.g., ≥97% w/w pure by HPLC, e.g., ≥98% w/w pure by HPLC, e.g., ≥99% w/w pure by HPLC.

Accordingly, provided is Crystalline Form G (also called Form G herein) of trans sodium crocetinate (Formula I above). Further provided is Crystalline Form G as follows:
G.1 Crystalline Form G, wherein the Crystalline Form G exhibits an XRPD pattern comprising 2-theta (°) values 4.3, 8.5, 15.6, and 17.3, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. Or, for instance, Crystalline Form G, wherein the Crystalline Form G exhibits an XRPD pattern comprising d-spacing (Å) values of 20.6, 10.4, 5.7, and 5.1.
G.2 Crystalline Form G or G.1, wherein the Crystalline Form G exhibits an XRPD pattern comprising 2-theta (°) values of 4.3, 8.5, 15.6, 17.3, 18.2, and 19.6, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. Or, for instance, Crystalline Form G or G.1, wherein the Crystalline Form G exhibits an XRPD pattern comprising d-spacing (Å) values of 20.6, 10.4, 5.7, 5.1, 4.9, and 4.5.
G.3 Any of Crystalline Form G, G.1, or G.2, wherein the Crystalline Form G exhibits an XRPD pattern comprising at least three, e.g., at least four, e.g., at least five, e.g., at least six, e.g., at least ten, 2-theta (°) values selected from the group consisting of 4.3, 8.5, 15.6, 17.3, 18.2, 19.6, 23.8, 24.3, 25.0, 28.8, 30.9, 34.3, and 38.6, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
G.4 Any of Crystalline Form G or G.1-G.3, wherein the Crystalline Form G exhibits an XRPD pattern comprising 2-theta (°) values of 4.3, 8.5, 15.6, 17.3, 18.2, 19.6, 23.8, 24.3, 25.0, 28.8, 30.9, 34.3, and 38.6, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
G.5 Any of Crystalline Form G or G.1-G.4, wherein the Crystalline Form G exhibits an XRPD pattern comprising at least three, e.g., at least four, e.g., at least five, e.g., at least six, e.g., at least ten, 2-theta (°) values selected from the group consisting of 4.28, 8.48, 15.64, 17.34, 18.24, 19.58, 23.84, 24.30, 24.96, 28.84, 30.88, 34.26, and 38.64, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
G.6 Any of Crystalline Form G or G.1-G.5, wherein the Crystalline Form G exhibits an XRPD pattern comprising 2-theta (°) values of 4.28, 8.48, 15.64, 17.34, 18.24, 19.58, 23.84, 24.30, 24.96, 28.84, 30.88, 34.26, and 38.64, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
G.7 Any of Crystalline Form G or G.1-G.6, wherein the Crystalline Form G exhibits an XRPD pattern comprising at least three, e.g., at least four, e.g., at least five, e.g., at least six, e.g., at least ten, d-spacing (Å) values selected from the group consisting of 20.6, 10.4, 5.7, 5.1, 4.9, 4.5, 3.7, 3.6, 3.1, 2.9, 2.6, and 2.3.
G.8 Any of Crystalline Form G or G.1-G.7, wherein the Crystalline Form G exhibits an XRPD pattern comprising d-spacing (Å) values of 20.6, 10.4, 5.7, 5.1, 4.9, 4.5, 3.7, 3.6, 3.1, 2.9, 2.6, and 2.3.
G.9 Any of Crystalline Form G or G.1-G.8, wherein the Crystalline Form G exhibits an XRPD pattern comprising at least three, e.g., at least four, e.g., at least five, e.g., at least six, e.g., at least ten, d-spacing (Å) values selected from the group consisting of 20.63, 10.42, 5.66, 5.11, 4.86, 4.53, 3.73, 3.66, 3.56, 3.09, 2.89, 2.62, and 2.33.
G.10 Any of Crystalline Form G or G.1-G.9, wherein the Crystalline Form G exhibits an XRPD pattern comprising d-spacing (Å) values of 20.63, 10.42, 5.66, 5.11, 4.86, 4.53, 3.73, 3.66, 3.56, 3.09, 2.89, 2.62, and 2.33.
G.11 Any of Crystalline Form G or G.1-G.10, wherein the Crystalline Form G exhibits an XRPD pattern comprising at least three, e.g., at least four, e.g., at least five, e.g., at least six, e.g., at least ten, d-spacing (Å) values selected from the group consisting of 20.628, 10.418, 5.661, 5.110, 4.860, 4.530, 3.729, 3.660, 3.565, 3.093, 2.893, 2.615, and 2.328.
G.12 Any of Crystalline Form G or G.1-G.11, wherein the Crystalline Form G exhibits an XRPD pattern comprising d-spacing (Å) values of 20.628, 10.418, 5.661, 5.110, 4.860, 4.530, 3.729, 3.660, 3.565, 3.093, 2.893, 2.615, and 2.328.
G.13 Any of Crystalline Form G or G.1-G.12, wherein the Crystalline Form G exhibits an XRPD pattern comprising at least three, e.g., at least four, e.g., at least five, e.g., at least six, e.g., at least ten, 2-theta (°) values selected from those set forth in Table G below:

TABLE G
Form G

| °2θ | Relative Intensity | d-spacing value (Å) |
|---|---|---|
| 4.28 | 100 | 20.6281 |
| 8.48 | 40 | 10.4184 |
| 15.64 | 9 | 5.6613 |
| 17.34 | 34 | 5.1099 |
| 18.24 | 6 | 4.8597 |
| 19.58 | 7 | 4.5301 |
| 23.84 | 8 | 3.7293 |
| 24.30 | 12 | 3.6598 |
| 24.96 | 11 | 3.5645 |
| 28.84 | 8 | 3.0932 |
| 30.88 | 9 | 2.8933 |
| 34.26 | 8 | 2.6152 |
| 38.64 | 10 | 2.3282 |

e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
G.14 Any of Crystalline Form G or G.1-G.13, wherein the Crystalline Form G exhibits an XRPD pattern comprising the 2-theta (°) values set forth in Table G in Crystalline Form G.13, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
G.15 Any of Crystalline Form G or G.1-G.14, wherein the Crystalline Form G exhibits an XRPD pattern comprising at least three, e.g., at least four, e.g., at least five, e.g., at least six, e.g., at least ten, d-spacing (Å) values selected from those set forth in Table G in Crystalline Form G.13.
G.16 Any of Crystalline Form G or G.1-G.15, wherein the Crystalline Form G exhibits an XRPD pattern comprising the d-spacing (Å) values set forth in Table G in Crystalline Form G.13.
G.17 Any of Crystalline Form G or G.1-G.16, wherein the Crystalline Form G exhibits an XRPD pattern comprising at least four, e.g., at least six, e.g., at least ten, 2-theta (°) values selected from those set forth in Table GG below:

TABLE GG
Form G

| °2θ | Relative Intensity | d-spacing value (Å) |
|---|---|---|
| 4.3 | 100 | 20.6281 |
| 8.5 | 40 | 10.4184 |
| 15.6 | 9 | 5.6613 |
| 17.3 | 34 | 5.1099 |
| 18.2 | 6 | 4.8597 |
| 19.6 | 7 | 4.5301 |
| 23.8 | 8 | 3.7293 |
| 24.3 | 12 | 3.6598 |
| 25.0 | 11 | 3.5645 |
| 28.8 | 8 | 3.0932 |
| 30.9 | 9 | 2.8933 |
| 34.3 | 8 | 2.6152 |
| 38.6 | 10 | 2.3282 |

e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
G.18 Any of Crystalline Form G or G.1-G.17, wherein the Crystalline Form G exhibits an XRPD pattern comprising the 2-theta (°) values set forth in Table GG in Crystalline Form G.17, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
G.19 Any of Crystalline Form G or G.1-G.18, wherein the Crystalline Form G exhibits an XRPD pattern comprising at least four, e.g., at least six, e.g., at least ten, d-spacing (Å) values selected from those set forth in Table GG in Crystalline Form G.17.
G.20 Any of Crystalline Form G or G.1-G.19, wherein the Crystalline Form G exhibits an XRPD pattern comprising the d-spacing (Å) values set forth in Table GG in Crystalline Form G.17.
G.21 Any of Crystalline Form G or G.1-G.20, wherein the Crystalline Form G exhibits an XRPD pattern comprising characteristic 2-theta (°) values selected from any of the 2-theta (°) values set forth in any of Crystalline Form G or G.1-G.20 above, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
G.22 Any of Crystalline Form G or G.1-G.21, wherein the Crystalline Form G exhibits an XRPD pattern comprising representative 2-theta (°) values selected from any of the 2-theta (°) values set forth in any of Crystalline Form G or G.1-G.20 above, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
G.23 Any of Crystalline Form G or G.1-G.22, wherein the Crystalline Form G exhibits an XRPD pattern comprising characteristic d-spacing (Å) values selected from any of the d-spacing (Å) values set forth in any of Crystalline Form G or G.1-G.20 above.
G.24 Any of Crystalline Form G or G.1-G.23, wherein the Crystalline Form G exhibits an XRPD pattern comprising representative d-spacing (Å) values selected from any of the d-spacing (Å) values set forth in any of Crystalline Form G or G.1-G.20 above.
G.25 Any of Crystalline Form G or G.1-G.24, wherein the Crystalline Form G exhibits an XRPD pattern comprising at least three, e.g., at least five, e.g., at least ten, e.g., all, of the peaks of the XRPD shown in FIG. 40, FIG. 41, and/or FIG. 48, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
G.26 Any of Crystalline Form G or G.1-G.25, wherein the Crystalline Form G exhibits an XRPD pattern comprising the characteristic peaks of the XRPD shown in FIG. 40, FIG. 41, and/or FIG. 48, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
G.27 Any of Crystalline Form G or G.1-G.26, wherein the Crystalline Form G exhibits an XRPD pattern comprising the representative peaks of the XRPD shown in FIG. 40, FIG. 41, and/or FIG. 48, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
G.28 Any of Crystalline Form G or G.1-G.27, wherein the Crystalline Form G exhibits an XRPD corresponding to FIG. 40, FIG. 41, and/or FIG. 48, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
G.29 Any of Crystalline Form G or G.1-G.28, wherein the Crystalline Form G is made as described in any of Process 1 or 1.1 et seq. vide infra or as described in any of Process 3 or 3.1 et seq. vide infra.
G.30 Any of Crystalline Form G or G.1-G.29, wherein the Crystalline Form G is made as described in any of the examples that produce Crystalline Form G.
G.31 Any of Crystalline Form G or G.1-G.30, wherein any of the listed 2-theta (°) values of an XRPD pattern have an acceptable deviation of ±0.2° G.32 Any of Crystalline Form G or G.1-G.31, wherein the 2-theta (°) values of an XRPD pattern have an acceptable deviation of ±0.2°.
G.33 Any of Crystalline Form G or G.1-G.32, wherein the Crystalline Form G is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of the amorphous form.
G.34 Any of Crystalline Form G or G.1-G.33, wherein the Crystalline Form G is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of any other crystalline form.
G.35 Any of Crystalline Form G or G.1-G.34, wherein the Crystalline Form G is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of the amorphous form and any other crystalline form.
G.36 Any of Crystalline Form G or G.1-G.35, wherein the Crystalline Form G is formulated as described in any of Process 2 or 2.1 et seq. vide infra.
G.37 Any of Crystalline Form G or G.1-G.36, wherein the Crystalline Form G does not change on storage at 25° C. over 3 months.
G.38 Any of Crystalline Form G, G.1-G.32, G.36, or G.37, wherein the Crystalline Form G is in a mixture with another crystalline form and/or amorphous form of trans sodium crocetinate.
G.39 Any of Crystalline Form G or G.1-G.38, wherein the Crystalline Form G is ≥96% w/w pure by HPLC, e.g., ≥97% w/w pure by HPLC, e.g., ≥98% w/w pure by HPLC, e.g., ≥99% w/w pure by HPLC.

Also provided is Crystalline Form J (also called Form J herein) of trans sodium crocetinate (Formula I above). Further provided is Crystalline Form J as follows:
J.1 Crystalline Form J, wherein the Crystalline Form J exhibits an XRPD pattern comprising 2-theta (°) values 13.0, 15.0, 18.3, and 18.6, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. Or, for instance, Crystalline Form J, wherein the Crystalline Form J exhibits an XRPD pattern comprising d-spacing (Å) values of 6.8, 5.9, and 4.8.
J.2 Crystalline Form J or J.1, wherein the Crystalline Form J exhibits an XRPD pattern comprising 2-theta (°) values of 7.0, 13.0, 13.7, 13.9, 15.0, 18.3, and 18.6, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å. Or, for instance, Crystalline Form J or J.1, wherein the Crystalline Form J exhibits an XRPD pattern comprising d-spacing (Å) values of 12.6, 6.8, 6.4, 6.3, 5.9, and 4.8.
J.3 Any of Crystalline Form J, J.1, or J.2, wherein the Crystalline Form J exhibits an XRPD pattern comprising at least three, e.g., at least four, e.g., at least five, e.g., at least seven, e.g., at least ten, e.g., at least fifteen, e.g., at least twenty, e.g., at least twenty-five, e.g., at least thirty, e.g., at least thirty-five, e.g., at least forty, 2-theta (°) values selected from the group consisting of 7.0, 9.5, 10.4, 13.0, 13.7, 13.9, 15.0, 15.2, 15.8, 17.6, 18.3, 18.6, 19.1, 19.7, 21.0, 21.8, 22.1, 22.4, 23.7, 24.2, 25.1, 25.5, 26.2, 26.7, 27.2, 27.5, 28.0, 28.1, 28.7, 30.2, 30.4, 30.6, 31.2, 31.6, 32.0, 34.0, 34.4, 35.0, 35.3, 35.5, 36.6, 37.2, 38.4, and 38.8, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
J.4 Any of Crystalline Form J or J.1-J.3, wherein the Crystalline Form J exhibits an XRPD pattern comprising 2-theta (°) values of 7.0, 9.5, 10.4, 13.0, 13.7, 13.9, 15.0, 15.2, 15.8, 17.6, 18.3, 18.6, 19.1, 19.7, 21.0, 21.8, 22.1, 22.4, 23.7, 24.2, 25.1, 25.5, 26.2, 26.7, 27.2, 27.5, 28.0, 28.1, 28.7, 30.2, 30.4, 30.6, 31.2, 31.6, 32.0, 34.0, 34.4, 35.0, 35.3, 35.5, 36.6, 37.2, 38.4, and 38.8, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
J.5 Any of Crystalline Form J or J.1-J.4, wherein the Crystalline Form J exhibits an XRPD pattern comprising at least three, e.g., at least four, e.g., at least five, e.g., at least seven, e.g., at least ten, e.g., at least fifteen, e.g., at least twenty, e.g., at least twenty-five, e.g., at least thirty, e.g., at least thirty-five, e.g., at least forty, d-spacing (Å) values selected from the group consisting of 12.6, 9.3, 8.5, 6.8, 6.4, 6.3, 5.9, 5.8, 5.6, 5.0, 4.8, 4.7, 4.5, 4.2, 4.1, 4.0, 3.7, 3.6, 3.5, 3.4, 3.3, 3.2, 3.1, 3.0, 2.9, 2.8, 2.6, 2.5, 2.4, and 2.3.
J.6 Any of Crystalline Form J or J.1-J.5, wherein the Crystalline Form J exhibits an XRPD pattern comprising d-spacing (Å) values of 12.6, 9.3, 8.5, 6.8, 6.4, 6.3, 5.9, 5.8, 5.6, 5.0, 4.8, 4.7, 4.5, 4.2, 4.1, 4.0, 3.7, 3.6, 3.5, 3.4, 3.3, 3.2, 3.1, 3.0, 2.9, 2.8, 2.6, 2.5, 2.4, and 2.3.
J.7 Any of Crystalline Form J or J.1-J.6, wherein the Crystalline Form J exhibits an XRPD pattern comprising at least three, e.g., at least four, e.g., at least five, e.g., at least seven, e.g., at least ten, e.g., at least fifteen, e.g., at least twenty, e.g., at least twenty-five, e.g., at least thirty, e.g., at least thirty-five, e.g., at least forty, d-spacing (Å) values selected from the group consisting of 12.64, 9.31, 8.50, 6.79, 6.45, 6.34, 5.88, 5.84, 5.62, 5.03, 4.84, 4.76, 4.65, 4.50, 4.23, 4.07, 4.01, 3.96, 3.75, 3.68, 3.55, 3.49, 3.40, 3.34, 3.28, 3.24, 3.19, 3.17, 3.10, 2.95, 2.94, 2.92, 2.86, 2.83, 2.80, 2.64, 2.60, 2.56, 2.54, 2.53, 2.45, 2.41, 2.34, and 2.32.
J.8 Any of Crystalline Form J or J.1-J.7, wherein the Crystalline Form J exhibits an XRPD pattern comprising d-spacing (k) values of 12.64, 9.31, 8.50, 6.79, 6.45, 6.34, 5.88, 5.84, 5.62, 5.03, 4.84, 4.76, 4.65, 4.50, 4.23, 4.07, 4.01, 3.96, 3.75, 3.68, 3.55, 3.49, 3.40, 3.34, 3.28, 3.24, 3.19, 3.17, 3.10, 2.95, 2.94, 2.92, 2.86, 2.83, 2.80, 2.64, 2.60, 2.56, 2.54, 2.53, 2.45, 2.41, 2.34, and 2.32.
J.9 Any of Crystalline Form J or J.1-J.8, wherein the Crystalline Form J exhibits an XRPD pattern comprising at least three, e.g., at least four, e.g., at least five, e.g., at least seven, e.g., at least ten, e.g., at least fifteen, e.g., at least twenty, e.g., at least twenty-five, e.g., at least thirty, e.g., at least thirty-five, e.g., at least forty, d-spacing (Å) values selected from the group consisting of 12.642, 9.313, 8.504, 6.790, 6.447, 6.344, 5.885, 5.836, 5.621, 5.028, 4.839, 4.758, 4.654, 4.496, 4.227, 4.072, 4.012, 3.957, 3.748, 3.681, 3.552, 3.490, 3.399, 3.335, 3.281, 3.239, 3.185, 3.174, 3.104, 2.954, 2.942, 2.919, 2.865, 2.826, 2.796, 2.638, 2.604, 2.565, 2.559, 2.525, 2.451, 2.412, 2.343, and 2.317.
J.10 Any of Crystalline Form J or J.1-J.9, wherein the Crystalline Form J exhibits an XRPD pattern comprising d-spacing (k) values of 12.642, 9.313, 8.504, 6.790, 6.447, 6.344, 5.885, 5.836, 5.621, 5.028, 4.839, 4.758, 4.654, 4.496, 4.227, 4.072, 4.012, 3.957, 3.748, 3.681, 3.552, 3.490, 3.399, 3.335, 3.281, 3.239, 3.185, 3.174, 3.104, 2.954, 2.942, 2.919, 2.865, 2.826, 2.796, 2.638, 2.604, 2.565, 2.559, 2.525, 2.451, 2.412, 2.343, and 2.317.
J.11 Any of Crystalline Form J or J.1-J.10, wherein the Crystalline Form J exhibits an XRPD pattern comprising at least three, e.g., at least four, e.g., at least five, e.g., at least seven, e.g., at least ten, e.g., at least fifteen, e.g., at least twenty, e.g., at least twenty-five, e.g., at least thirty, e.g., at least thirty-five, e.g., at least forty, 2-theta (°) values selected from those set forth in Table J below:

TABLE J

| °2θ | Relative Intensity | d-spacing value (Å) |
|---|---|---|
| 7.0 | 3 | 12.6421 |
| 9.5 | 1 | 9.3128 |
| 10.4 | 1 | 8.5044 |
| 13.0 | 76 | 6.7898 |
| 13.7 | 5 | 6.4468 |
| 13.9 | 8 | 6.3436 |
| 15.0 | 16 | 5.8847 |
| 15.2 | 2 | 5.8360 |
| 15.8 | 3 | 5.6212 |
| 17.6 | 4 | 5.0279 |
| 18.3 | 11 | 4.8394 |
| 18.6 | 20 | 4.7575 |
| 19.1 | 4 | 4.6538 |
| 19.7 | 4 | 4.4962 |
| 21.0 | 44 | 4.2266 |
| 21.8 | 97 | 4.0723 |
| 22.1 | 11 | 4.0119 |
| 22.4 | 8 | 3.9574 |
| 23.7 | 5 | 3.7478 |
| 24.2 | 21 | 3.6807 |
| 25.1 | 18 | 3.5517 |
| 25.5 | 1 | 3.4899 |
| 26.2 | 100 | 3.3985 |
| 26.7 | 2 | 3.3354 |
| 27.2 | 4 | 3.2811 |
| 27.5 | 6 | 3.2392 |
| 28.0 | 3 | 3.1852 |
| 28.1 | 2 | 3.1743 |
| 28.7 | 7 | 3.1043 |
| 30.2 | 5 | 2.9543 |
| 30.4 | 1 | 2.9415 |
| 30.6 | 6 | 2.9187 |
| 31.2 | 5 | 2.8648 |
| 31.6 | 6 | 2.8256 |
| 32.0 | 2 | 2.7961 |
| 34.0 | 2 | 2.6383 |
| 34.4 | 1 | 2.6041 |
| 35.0 | 4 | 2.5648 |
| 35.3 | 2 | 2.5392 |
| 35.5 | 1 | 2.5254 |
| 36.6 | 2 | 2.4507 |
| 37.2 | 1 | 2.4120 |
| 38.4 | 2 | 2.3431 |
| 38.8 | 3 | 2.3165, |

e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
J.12 Any of Crystalline Form J or J.1-J.11, wherein the Crystalline Form J exhibits an XRPD pattern comprising the 2-theta (°) values set forth in Table J in Crystalline Form J.11, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
J.13 Any of Crystalline Form J or J.1-J.12, wherein the Crystalline Form J exhibits an XRPD pattern comprising at least three, e.g., at least four, e.g., at least five, e.g., at least seven, e.g., at least ten, e.g., at least fifteen, e.g., at least twenty, e.g., at least twenty-five, e.g., at least thirty, e.g., at least thirty-five, e.g., at least forty, d-spacing (Å) values selected from those set forth in Table J in Crystalline Form J.11.
J.14 Any of Crystalline Form J or J.1-J.13, wherein the Crystalline Form J exhibits an XRPD pattern comprising the d-spacing (Å) values set forth in Table J in Crystalline Form J.13.
J.15 Any of Crystalline Form J or J.1-J.14, wherein the Crystalline Form J exhibits an XRPD pattern comprising characteristic 2-theta (°) values selected from any of the 2-theta (°) values set forth in any of Crystalline Form J or J.1-J.14 above, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
J.16 Any of Crystalline Form J or J.1-J.16, wherein the Crystalline Form J exhibits an XRPD pattern comprising representative 2-theta (°) values selected from any of the 2-theta (°) values set forth in any of Crystalline Form J or J.1-J.14 above, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
J.17 Any of Crystalline Form J or J.1-J.16, wherein the Crystalline Form J exhibits an XRPD pattern comprising characteristic d-spacing (Å) values selected from any of the d-spacing (Å) values set forth in any of Crystalline Form J or J.1-J.14 above.
J.18 Any of Crystalline Form J or J.1-J.17, wherein the Crystalline Form J exhibits an XRPD pattern comprising representative d-spacing (Å) values selected from any of the d-spacing (Å) values set forth in any of Crystalline Form J or J.1-J.14 above.
J.19 Any of Crystalline Form J or J.1-J.18, wherein the Crystalline Form J exhibits an XRPD pattern comprising at least three, e.g., at least four, e.g., at least five, e.g., at least seven, e.g., at least ten, e.g., at least fifteen, e.g., at least twenty, e.g., at least twenty-five, e.g., at least thirty, e.g., at least thirty-five, e.g., at least forty, e.g., all, of the peaks of the XRPD shown in FIG. 39, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
J.20 Any of Crystalline Form J or J.1-J.19, wherein the Crystalline Form J exhibits an XRPD pattern comprising the characteristic peaks of the XRPD shown in FIG. 39, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
J.21 Any of Crystalline Form J or J.1-J.20, wherein the Crystalline Form J exhibits an XRPD pattern comprising the representative peaks of the XRPD shown in FIG. 39, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
J.22 Any of Crystalline Form J or J.1-J.21, wherein the Crystalline Form J exhibits an XRPD corresponding to FIG. 39, e.g., wherein the XRPD is obtained using an incident beam of Cu radiation, e.g., wherein the XRPD is obtained using radiation of wavelength 1.5406 Å, e.g., wherein the XRPD is obtained with a Cu x-ray source and the x-ray wavelength is 1.5406 Å.
J.23 Any of Crystalline Form J or J.1-J.22, wherein the Crystalline Form J exhibits a differential scanning calorimetry (DSC) thermogram comprising an endotherm between 50-60° C., e.g., between 52-56° C. For instance, any of Crystalline Form J or J.1-J.22, wherein the Crystalline Form J exhibits a differential scanning calorimetry (DSC) thermogram comprising an endotherm at 54° C.
J.24 Any of Crystalline Form J or J.1-J.23, wherein the Crystalline Form J exhibits a differential scanning calorimetry (DSC) thermogram as shown in FIG. 42.
J.25 Any of Crystalline Form J or J.1-J.24, wherein the Crystalline Form J exhibits a thermogravimetric analysis (TGA) thermogram comprising weight loss between start (e.g., 25° C.) and 100° C., e.g., between start (e.g., 25° C.) and 100° C., e.g., a weight loss of 0-1 weight %, e.g., a weight loss of 0.1-0.5 weight %, e.g., a weight loss of 0.3 weight %.
J.26 Any of Crystalline Form J or J.1-J.25, wherein the Crystalline Form J exhibits a thermogravimetric analysis (TGA) thermogram as shown in FIG. 42.
J.27 Any of Crystalline Form J or J.1-J.26, wherein the Crystalline Form J is made as described in any of Process 1 or 1.1 et seq. vide infra or as described in any of Process 3 or 3.1 et seq. vide infra.
J.28 Any of Crystalline Form J or J.1-J.27, wherein the Crystalline Form J is made as described in any of the examples that produce Crystalline Form J.
J.29 Any of Crystalline Form J or J.1-J.28, wherein any of the listed 2-theta (°) values of an XRPD pattern have an acceptable deviation of ±0.2°.
J.30 Any of Crystalline Form J or J.1-J.29, wherein the 2-theta (°) values of an XRPD pattern have an acceptable deviation of ±0.2°.
J.31 Any of Crystalline Form J or J.1-J.30, wherein the Crystalline Form J is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of the amorphous form.
J.32 Any of Crystalline Form J or J.1-J.31, wherein the Crystalline Form J is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of any other crystalline form.
J.33 Any of Crystalline Form J or J.1-J.32, wherein the Crystalline Form J is free or substantially free of any other form, e.g., comprises less than 20 weight %, e.g., less than 15 weight %, e.g., less than 10 weight %, e.g., less than 5 weight %, e.g., less than 3 weight %, e.g., less than 2 weight %, e.g., less than 1 weight %, e.g., less than 0.1 weight %, of the amorphous form and any other crystalline form.
J.34 Any of Crystalline Form J or J.1-J.33, wherein the Crystalline Form J is formulated as described in any of Process 2 or 2.1 et seq. vide infra.
J.35 Any of Crystalline Form J or J.1-J.34, wherein Crystalline Form J is unsolvated (including unhydrated). Any of Crystalline Form J or J.1-J.34, wherein Crystalline Form J is a non-solvate non-hydrate.
J.36 Any of Crystalline Form J, J.1-J.30, J.34, or J.35, wherein the Crystalline Form J is in a mixture with another crystalline form and/or amorphous form of trans sodium crocetinate.
J.37 Any of Crystalline Form J or J.1-J.36, wherein the Crystalline Form J is ≥96% w/w pure by HPLC, e.g., ≥97% w/w pure by HPLC, e.g., ≥98% w/w pure by HPLC, e.g., ≥99% w/w pure by HPLC.

Further provided is trans sodium crocetinate in crystalline form, wherein the crystalline form exhibits an XRPD pattern corresponding to any pattern disclosed herein (for instance, corresponding to any pattern in any of the figures).

Further provided is trans sodium crocetinate in a stable crystalline form. For instance, wherein the crystalline form remains unchanged under various heating and/or humidity conditions. For instance, wherein the crystalline form remains unchanged after exposure to 50% RH for 3 hours or 13 hours. Or, for instance, wherein the crystalline form remains unchanged after DVS analysis where the sample is exposed up to 95% relative humidity (RH) and down to 5% RH. Or, for instance, wherein the crystalline form remains unchanged for up to 4 weeks at 25° C./60% relative humidity (RH) and/or for up to 4 weeks at 40° C./75% RH.

Further provided is a crystalline form having the purity (e.g., by HPLC) and/or water assay (e.g., by Karl-Fischer) and/or sodium assay (e.g., by Flame Atomic Absorption Spectrometry (FAAS) and/or elemental analysis) as described in any of the examples. For instance, any of the crystalline forms described herein wherein the sodium assay (e.g., by Flame Atomic Absorption Spectrometry (FAAS) and/or elemental analysis) is ≥11.7%, e.g., is ≥12-15%, e.g., ≥12-14%.

Further provided is a process (Process 1) for making trans sodium crocetinate (Formula I) in crystalline form, e.g., for making any of Crystalline Forms A-G or J vide supra (e.g., any of Crystalline Form A or A.1 et seq. or any of Crystalline Form B or B.1 et seq. or any of Crystalline Form C or C.1 et seq. or any of Crystalline Form D or D.1 et seq. or any of Crystalline Form E or E.1 et seq. or any of Crystalline Form F or F.1 et seq. or any of Crystalline Form G or G.1 et seq. or any of Crystalline Form J or J.1 et seq.). Further provided is Process 1 as follows:
1.1 Process 1, wherein the process comprises mixing trans sodium crocetinate with n-butanol and ethanol. For instance, Process 1, wherein the process comprises mixing trans sodium crocetinate with n-butanol, ethanol, and water. For instance, Process 1, wherein the process comprises mixing trans sodium crocetinate with n-butanol, ethanol, water, and sodium hydroxide. For instance, Process 1, wherein the process comprises mixing trans sodium crocetinate with n-butanol, ethanol, and 30% sodium hydroxide in water. For instance, Process 1, wherein the process comprises crystallizing trans sodium crocetinate from ethanol, n-butanol, and water. For instance, Process 1, wherein the process comprises crystallizing trans sodium crocetinate from ethanol, n-butanol, water, and sodium hydroxide. For instance, Process 1, wherein the process comprises crystallizing trans sodium crocetinate from 6:1 to 1:1 n-butanol:ethanol (e.g., 4:1 n-butanol:ethanol) and optionally with water or optionally with water and sodium hydroxide (e.g., at room temperature or below room temperature). For instance, Process 1, wherein the process comprises crystallizing trans sodium crocetinate from a mixture of n-butanol, ethanol, and water, wherein the mixture comprises 6:1 to 1:1 n-butanol:ethanol (e.g., 4:1 n-butanol:ethanol) and 30:1 to 10:1 water:ethanol (e.g., 20:1 to 15:1 water:ethanol) and optionally with sodium hydroxide (e.g., at room temperature or below room temperature).
1.2 Process 1.1, wherein the process comprises removing n-butanol and ethanol leaving mostly water. For instance, Process 1.1, wherein the process comprises heating (e.g., heating to an external temperature of 40-60° C., e.g., heating to an external temperature of 50° C.) the mixture to remove n-butanol and ethanol, e.g., by distillation (e.g., 70-200 mbar).
1.3 Process 1.1 or 1.2, wherein the process comprises adding ethanol, e.g., adding ethanol slowly. For instance, Process 1.1 or 1.2, wherein the process comprises adding ethanol, e.g., adding ethanol slowly, while heating (e.g., heating to an internal temperature of 40-60° C., e.g., heating to an internal temperature of 50° C.).
1.4 Any of Process 1.1-1.3, wherein the process comprises cooling the mixture (e.g., cooling the mixture to ambient temperature, e.g., cooling the internal temperature to 20° C.). For instance, any of Process 1.1-1.3, wherein the process comprises cooling the mixture over at least 30 minutes (e.g., cooling the mixture to ambient temperature, e.g., cooling the internal temperature to 20° C.).
1.5 Any of Process 1.1-1.4, wherein the process comprises filtering the mixture.
1.6 Any of Process 1.1-1.5, wherein the process comprises rinsing the filter cake with ethanol and water (e.g., a 1:1 mixture of ethanol and water).
1.7 Any of Process 1.1-1.6, wherein the process comprises drying the filter cake (e.g., drying the filter cake to a dry content of ≥50% w/w, e.g., drying the filter cake to a dry content of ≥50% w/w and the solid is transferable).
1.8 Any of Process 1.1-1.7, wherein the process comprises stirring (e.g., in a reactor) the solid (i.e., the filter cake) with ethanol and water (e.g., a 1:1 mixture of ethanol and water). Any of Process 1.1-1.7, wherein the process comprises stirring the solid with a 1:1 mixture of ethanol and water at ambient temperature (e.g., at an internal temperature of 20° C.) for at least 1 hour.
1.9 Any of Process 1 or 1.1-1.8, wherein the process comprises filtering the mixture.
1.10 Any of Process 1.1-1.9, wherein the process comprises rinsing the filter cake with ethanol and water (e.g., a 1:1 mixture of ethanol and water).
1.11 Any of Process 1 or 1.1-1.10, wherein the process comprises drying the filter cake (e.g., drying the filter cake to a dry content of ≥50% w/w, e.g., drying the filter cake to a dry content of ≥50% w/w and the solid is transferable).
1.12 Any of Process 1 or 1.1-1.11, wherein the process comprises stirring (e.g., in a reactor) the solid (i.e., filter cake) with ethanol (e.g., at ambient temperature, e.g., at ambient temperature for at least 1 hour).
1.13 Any of Process 1 or 1.1-1.12, wherein the process comprises filtering the mixture.
1.14 Any of Process 1 or 1.1-1.13, wherein the process comprises rinsing the filter cake with ethanol.
1.15 Any of Process 1 or 1.1-1.14, wherein the process comprises drying the filter cake (e.g., drying the filter cake to a dry content of ≥98% w/w).
1.16 Any of Process 1 or 1.1-1.15, wherein the process comprises drying the solid (i.e., the filter cake) on a rotary evaporator.
1.17 Any of Process 1 or 1.1-1.16, wherein the trans sodium crocetinate has ≤0.4% w/w ethanol (e.g., as determined by nuclear magnetic resonance (NMR) spectroscopy).
1.18 Any of Process 1 or 1.1-1.17, wherein the trans sodium crocetinate is optionally milled.
1.19 Any of Process 1 or 1.1-1.18, wherein the process comprises seeding (e.g., seeding a solution or slurry) with crystals of the desired form. For instance, any of Process 1 or 1.1-1.18, wherein the process comprises seeding (e.g., seeding a solution or slurry) with any crystalline form described herein. For instance, any of Process 1 or 1.1-1.18, wherein the process comprises seeding (e.g., seeding a solution or slurry) with Crystalline Form A, e.g., any of Crystalline Form A or A.1 et seq. Or, for instance, any of Process 1 or 1.1-1.18, wherein the process comprises seeding (e.g., seeding a solution or slurry) with Crystalline Form B, e.g., any of Crystalline Form B or B.1 et seq. Or, for instance, any of Process 1 or 1.1-1.18, wherein the process comprises seeding (e.g., seeding a solution or slurry) with Crystalline Form F, e.g., any of Crystalline Form F or F.1 et seq.
1.20 Any of Process 1 or 1.1-1.19, wherein the process comprises isolating the trans sodium crocetinate crystal. For instance, any of Process 1 or 1.1-1.19, wherein the process comprising isolating any crystalline form described herein.
1.21 Any of Process 1 or 1.1-1.20, wherein the process comprises isolating any of Crystalline Form A or A.1 et seq. Or, any of Process 1 or 1.1-1.20, wherein the process comprises isolating any of Crystalline Form B or B.1 et seq.
1.22 Any of Process 1 or 1.1-1.20, wherein the process comprises isolating any of Crystalline Form F or F.1 et seq.
1.23 A crystal comprising trans sodium crocetinate made by any of Process 1 et seq.
1.24 Any of Crystalline Forms A-G or J vide supra (e.g., any of Crystalline Form A or A.1 et seq. or any of Crystalline Form B or B.1 et seq. or any of Crystalline Form C or C.1 et seq. or any of Crystalline Form D or D.1 et seq. or any of Crystalline Form E or E.1 et seq. or any of Crystalline Form F or F.1 et seq. or any of Crystalline Form G or G.1 et seq. or any of Crystalline Form J or J.1 et seq.), wherein the crystal is made by any of Process 1 et seq.
1.25 Any of Process 1 et seq., wherein the process is as described in any of the synthetic examples herein.

Further provided is a crystal made by any of Process 1 et seq.

Further provided is a pharmaceutical composition (Composition 1a) (e.g., a pharmaceutical composition for injection), wherein the pharmaceutical composition comprises trans sodium crocetinate (Formula I) in crystalline form, e.g., any of Crystalline Forms A-G or J vide supra (e.g., any of Crystalline Form A or A.1 et seq. or any of Crystalline Form B or B.1 et seq. or any of Crystalline Form C or C.1 et seq. or any of Crystalline Form D or D.1 et seq. or any of Crystalline Form E or E.1 et seq. or any of Crystalline Form F or F.1 et seq. or any of Crystalline Form G or G.1 et seq. or any of Crystalline Form J or J.1 et seq.). For instance, further provided is a pharmaceutical composition, wherein the pharmaceutical composition comprises Crystalline Form A (e.g., any of Crystalline Form A or A.1 et seq.) of trans sodium crocetinate. Or, for instance, further provided is a pharmaceutical composition, wherein the pharmaceutical composition comprises Crystalline Form B (e.g., any of Crystalline Form B or B.1 et seq.) of trans sodium crocetinate. Or, for instance, further provided is a pharmaceutical composition, wherein the pharmaceutical composition comprises Crystalline Form F (e.g., any of Crystalline Form F or F.1 et seq.) of trans sodium crocetinate. For instance, further provided is a pharmaceutical composition (Composition 1a) (e.g., a pharmaceutical composition for injection), wherein the pharmaceutical composition comprises trans sodium crocetinate (Formula I) in crystalline form, e.g., any of Crystalline Forms A-G or J vide supra (e.g., any of Crystalline Form A or A.1 et seq. or any of Crystalline Form B or B.1 et seq. or any of Crystalline Form C or C.1 et seq. or any of Crystalline Form D or D.1 et seq. or any of Crystalline Form E or E.1 et seq. or any of Crystalline Form F or F.1 et seq. or any of Crystalline Form G or G.1 et seq. or any of Crystalline Form J or J.1 et seq.) and a pharmaceutically acceptable carrier or diluent.

Further provided is a pharmaceutical composition (Composition 1b) (e.g., a pharmaceutical composition for injection) as described in any of U.S. Pat. Nos. 7,351,844, 7,759,506, 8,030,350, 8,293,804, 8,974,822, and 10,130,689, U.S. Patent Publication Nos. 2019/0083439 and 2021/0244698, and International Publication No. WO 2021/202735, each of which is hereby incorporated by reference in its entirety, wherein the pharmaceutical composition comprises trans sodium crocetinate (Formula I) in crystalline form, e.g., any of Crystalline Forms A-G or J vide supra (e.g., any of Crystalline Form A or A.1 et seq. or any of Crystalline Form B or B.1 et seq. or any of Crystalline Form C or C.1 et seq. or any of Crystalline Form D or D.1 et seq. or any of Crystalline Form E or E.1 et seq. or any of Crystalline Form F or F.1 et seq. or any of Crystalline Form G or G.1 et seq. or any of Crystalline Form J or J.1 et seq.). For instance, further provided is a pharmaceutical composition, wherein the pharmaceutical composition comprises Crystalline Form A (e.g., any of Crystalline Form A or A.1 et seq.) of trans sodium crocetinate. Or, for instance, further provided is a pharmaceutical composition, wherein the pharmaceutical composition comprises Crystalline Form B (e.g., any of Crystalline Form B or B.1 et seq.) of trans sodium crocetinate. Or, for instance, further provided is a pharmaceutical composition, wherein the pharmaceutical composition comprises Crystalline Form F (e.g., any of Crystalline Form F or F.1 et seq.) of trans sodium crocetinate.

Further provided is Composition 1a or 1b, wherein the composition is for use in any of the methods described herein, e.g., for use in any of Method 1 vide infra.

Further provided is Composition 1a or 1b, wherein the composition exhibits an XRPD pattern of a crystalline form of trans sodium crocetinate, e.g., an XRPD pattern of any of Crystalline Forms A-G or J vide supra (e.g., any of Crystalline Form A or A.1 et seq. or any of Crystalline Form B or B.1 et seq. or any of Crystalline Form C or C.1 et seq. or any of Crystalline Form D or D.1 et seq. or any of Crystalline Form E or E.1 et seq. or any of Crystalline Form F or F.1 et seq. or any of Crystalline Form G or G.1 et seq. or any of Crystalline Form J or J.1 et seq.). For instance, an XRPD pattern as discussed in any of Crystalline Forms A-G or J vide supra (e.g., any of Crystalline Form A or A.1 et seq. or any of Crystalline Form B or B.1 et seq. or any of Crystalline Form C or C.1 et seq. or any of Crystalline Form D or D.1 et seq. or any of Crystalline Form E or E.1 et seq. or any of Crystalline Form F or F.1 et seq. or any of Crystalline Form G or G.1 et seq. or any of Crystalline Form J or J.1 et seq.).

Further provided is a process (Process 2) for preparing a pharmaceutical composition (e.g., a lyophilized pharmaceutical composition) comprising trans sodium crocetinate (Formula I), wherein the process comprises formulating a crystalline form of trans sodium crocetinate (e.g., any of Crystalline Forms A-G or J vide supra, e.g., formulating any of Crystalline Form A or A.1 et seq. or any of Crystalline Form B or B.1 et seq. or any of Crystalline Form C or C.1 et seq. or any of Crystalline Form D or D.1 et seq. or any of Crystalline Form E or E.1 et seq. or any of Crystalline Form F or F.1 et seq. or any of Crystalline Form G or G.1 et seq. or any of Crystalline Form J or J.1 et seq.). Further provided is Process 2 as follows:
2.1 Process 2, wherein the crystalline form of trans sodium crocetinate is Crystalline Form A or A.1 et seq.
2.2 Process 2, wherein the crystalline form of trans sodium crocetinate is Crystalline Form B or B.1 et seq.
2.3 Process 2, wherein the crystalline form of trans sodium crocetinate is Crystalline Form F or F.1 et seq.
2.4 Any of Process 2 or 2.1-2.3, wherein the process comprises dissolving the crystalline form in a liquid, e.g., dissolving the crystalline form in an aqueous solution.
2.5 Any of Process 2 or 2.1-2.4, wherein the process comprises mixing the crystalline form with a cyclodextrin (e.g., gamma-cyclodextrin).
2.6 Any of Process 2 or 2.1-2.5, wherein the process comprises mixing the crystalline form with mannitol (e.g., d-mannitol).
2.7 Any of Process 2 or 2.1-2.6, wherein the process comprises mixing the crystalline form with glycine.
2.8 Any of Process 2 or 2.1-2.7, wherein the process comprises lyophilizing the crystalline form (e.g., lyophilizing the mixture of the crystalline form, a cyclodextrin, mannitol, and glycine, e.g., lyophilizing an aqueous solution of the crystalline form, a cyclodextrin, mannitol, and glycine).
2.9 Any of Process 2 or 2.1-2.8, wherein the pharmaceutical composition has a pH of 8.0-8.2.
2.10 Any of Process 2 or 2.1-2.9, wherein the pharmaceutical composition has a pH of 8.2±0.5.
2.11 Any of Process 2 or 2.1-2.10, wherein the pharmaceutical composition is for injection, e.g., intravenous injection.
2.12 Any of Process 2 or 2.1-2.11, wherein the pharmaceutical composition is mixed (e.g., reconstituted) with an aqueous solution (e.g., saline or sterile water for injection) for administration.
2.13 Any of Process 2 or 2.1-2.12, wherein the process comprises mixing the crystalline form with a pharmaceutically acceptable diluent or carrier.
2.14 Any of Process 2 or 2.1-2.13, wherein the process comprises isolating the crystalline form (e.g., isolating any of Crystalline Form A or A. 1 et seq. or any of Crystalline Form B or B.1 et seq. or any of Crystalline Form F or F.1 et seq.) and mixing the crystalline form with a pharmaceutically acceptable diluent or carrier. For instance, any of Process 2 or 2.1-2.13, wherein the process comprises isolating the crystalline form (e.g., isolating any of Crystalline Form A or A.1 et seq. or any of Crystalline Form B or B. 1 et seq. or any of Crystalline Form F or F.1 et seq.) and mixing the crystalline form with a pharmaceutically acceptable liquid and optionally lyophilizing the mixture.
2.15 Any of Process 2 or 2.1-2.14, wherein the pharmaceutical composition exhibits an XRPD pattern corresponding to FIG. 50.

Further provided is a process (Process 3) for making trans sodium crocetinate (Formula I). Further provided is Process 3 as follows:
3.1 Process 3, wherein the process comprises reacting:

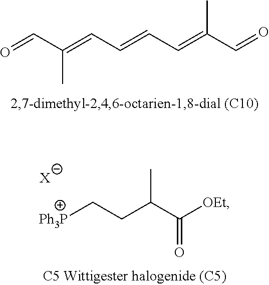

wherein X⁻ is Cl⁻ and/or Br⁻ (e.g., a mixture of Cl⁻ and Br⁻ or Br⁻ only).
3.2 Process 3 or 3.1, wherein the process comprises the following reaction:

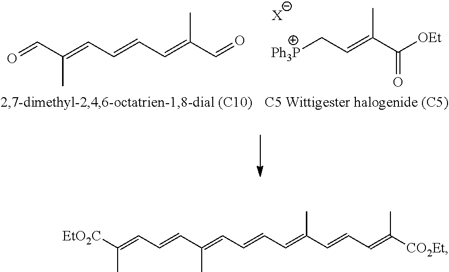

wherein X⁻ is Cl⁻ and/or Br⁻ (e.g., a mixture of Cl⁻ and Br⁻ or Br⁻ only).
3.3 Process 3.1 or 3.2, wherein the reaction occurs in acetonitrile and water.
3.4 Any of Process 3.1-3.3, wherein the reaction occurs with an alkali acetate base (e.g., NaOAc). For instance, any of Process 3.1-3.3, wherein this reaction occurs with excess (relative to C10) NaOAc. For instance, any of Process 3.1-3.3, wherein the reaction occurs with 3-6 equivalents (relative to C10) of NaOAc. For instance, any of Process 3.1-3.3, wherein the reaction occurs with 6 equivalents (relative to C10) of NaOAc. Isomerization rate is productive at 6 equivalents.
3.5 Any of Process 3.1-3.4, wherein the reaction occurs with excess (relative to C10) C5. For instance, any of Process 3.1-3.4, wherein the reaction occurs with 2 or more equivalents (relative to C10) of C5. For instance, any of Process 3.1-3.4, wherein the reaction occurs with 2-3 equivalents (relative to C10) of C5 (e.g., 2.4-2.5 equivalents, e.g., 2.4 equivalents).
3.6 Any of Process 3.1-3.5, wherein the reaction occurs with water (e.g., a small amount of water). For instance, any of Process 3.1-3.5, wherein the reaction occurs with less than 1 equivalent of water, e.g., 0.5 equivalents or less of water, e.g. 0.1 equivalent of water. Water allows for faster isomerization to the desired trans isomer, while reducing the viscous oily cis isomers. Water also improves reaction work-up by allowing more time for the cis-trans isomerization. An excess of water may cause C5 to be used up unproductively, thus beneficial results are observed with a small amount of water.
3.7 Any of Process 3.1-3.6, wherein the reaction is heated (e.g., heated to reflux, e.g., heated to 70-90° C., e.g., internal temperature 80° C. and/or jacket temperature 85° C.). For instance, any of Process 3.1-3.6, wherein the reaction is heated for 15-72 hours.
3.8 Any of Process 3.1-3.7, wherein water is added (e.g., 10 L/kg), optionally with stirring (e.g., for at least 12 hours) and/or cooling (e.g., to 60° C.).
3.9 Any of Process 3.1-3.8, wherein the phases are separated. For instance, any of process 3.1-3.8, wherein the phases are separated and the organic phase is kept.
3.10 Any of Process 3.1-3.9, wherein ethanol and water are added.
3.11 Process 3.10, wherein the mixture is filtered, optionally after being cooled (e.g., to 20° C.).
3.12 Process 3.11, wherein the product is washed with an alcoholic solvent (e.g., ethanol). For instance, Process 3.11, wherein the product is washed with 1:1 ethanol:water. Washing with 50% ethanol removes triphenylphosphine oxide.
3.13 Process 3.11 or 3.12, wherein the product is dried (e.g., on filter and/or rotovaporator) until dry content ≥98% w/w.
3.14 Any of Process 3 or 3.1-3.13, wherein the process comprises removing triphenyl phosphine oxide (e.g., by washing with an alcoholic solvent, e.g., ethanol, e.g., washing with 50% ethanol in water).
3.15 Any of Process 3 or 3.1-3.14, wherein the process comprises the following reaction:

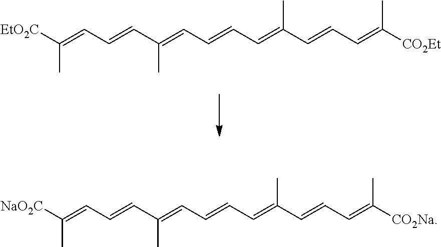

3.16 Process 3.15, wherein the reaction occurs in 1-butanol and ethanol (e.g., 1:1 n-butanol:ethanol, e.g., 10 L/kg 1-BuOH and 10 L/kg EtOH). Use of 1-butanol with ethanol improves conversion of the water insoluble diethyl ester due to its higher solubility achieved by inclusion of 1-butanol. In addition, it allows lowering of the process temperature. A lower process temperature (e.g., lower than reflux, e.g., 40-60° C., e.g., 50° C.) leads to lower levels of impurities. 1-butanol does not freely mix with water in the absence of ethanol, so 1-butanol is used as a co-solvent with ethanol.
3.17 Process 3.15 or 3.16, wherein the process comprises reacting the diethyl ester with a sodium base (e.g., NaOH and/or NaOEt).
3.18 Any of Process 3.15-3.17, wherein the process comprises reacting the diethyl ester with an excess of sodium base.
3.19 Any of Process 3.15-3.18, wherein the process comprises reacting the diethyl ester with 3-9 (e.g., 3-8) equivalents of sodium base.
3.20 Any of Process 3.15-3.19, wherein the process comprises reacting the diethyl ester with 3-4 equivalents of sodium base (e.g., NaOH). For instance, process 3.16 or 3.17, wherein the process comprises reacting the diethyl ester with 3.7-3.9 (e.g., 3.8) equivalents of sodium base (e.g., NaOH). In particular, an excess of 3-4 equivalents of sodium hydroxide in 1-butanol and ethanol allows trans sodium crocetinate to dissolve in an acceptable volume of solution.
3.21 Any of Process 3.15-3.20, wherein the reaction is heated, e.g., wherein the reaction is heated between 40-90° C. For instance, any of Process 3.15-3.20, wherein the reaction is heated, e.g., wherein the reaction is heated to reflux.
3.22 Any of Process 3.15-3.21, wherein the reaction is heated (e.g., for 24-72 hours) to an internal and/or external temperature of 40-60° C., e.g., an internal and/or external temperature of 50° C.
3.23 Any of Process 3.15-3.22, wherein the reaction is concentrated (e.g., under heating, e.g., heating to an external temperature of 40-60° C. (e.g., 50° C.) and 70-200 mbar). For instance, any of Process 3.15-3.22, wherein the mixture is concentrated to 10-13 L/kg, e.g., to 11 L/kg.
3.24 Process 3.23, wherein additional water (e.g., 20-40 L/kg, e.g., 30 L/kg) is added. Addition of water drives conversion.
3.25 Any of Process 3.15-3.24, wherein the reaction is heated (e.g., for at least 12 hours) to an external temperature of 40-60° C. (e.g., 50° C.).
3.26 Any of Process 3.15-3.25, wherein the reaction is filtered.
3.27 Any of Process 3.15-3.26, wherein the reaction is concentrated (e.g., under heating, e.g., heating to an external temperature of 40-60° C. (e.g., 50° C.) and 70-200 mbar). For instance, any of Process 3.15-3.26, wherein the reaction is concentrated but still allows full dissolution of trans sodium crocetinate. For instance, any of Process 3.15-3.26, wherein the mixture is concentrated to 10-13 L/kg, e.g., to 11 L/kg. Ethanol may be added during concentration to control foaming and/or as entrainer. Trans sodium crocetinate may precipitate during concentration.
3.28 Any of Process 3.15-3.27, wherein the process comprises removing n-butanol and ethanol leaving mostly water. For instance, Process 3.15-3.27, wherein the process comprises heating (e.g., heating to an external temperature of 40-60° C., e.g., heating to an external temperature of 50° C.) the mixture to remove n-butanol and ethanol, e.g., by distillation (e.g., 70-200 mbar). Additional ethanol may be added during removal of solvent. Trans sodium crocetinate may precipitate during removal of solvent.
3.29 Any of Process 3.15-3.28, wherein the process comprises cooling the mixture (e.g., cooling the mixture to ambient temperature, e.g., cooling the internal temperature to 20° C.). For instance, any of Process 3.15-3.28, wherein the process comprises cooling the mixture over at least 30 minutes (e.g., cooling the mixture to ambient temperature, e.g., cooling the internal temperature to 20° C.).
3.30 Any of Process 3.15-3.29, wherein the process comprises filtering the mixture.
3.31 Process 3.30, wherein the process comprises rinsing the filter cake with ethanol and water (e.g., a 1:1 mixture of ethanol and water).
3.32 Process 3.30 or 3.31, wherein the process comprises drying the filter cake (e.g., drying the filter cake to a dry content of ≥50% w/w, e.g., drying the filter cake to a dry content of ≥50% w/w and the solid is transferable).
3.33 Any of Process 3.15-3.33, wherein the process comprises stirring (e.g., in a reactor) the trans sodium crocetinate (e.g., the filter cake) with ethanol and water (e.g., a 1:1 mixture of ethanol and water) (e.g., a slurry wash). Any of Process 3.15-3.33, wherein the process comprises stirring the trans sodium crocetinate (e.g., the filter cake) with a 1:1 mixture of ethanol and water at ambient temperature (e.g., at an internal temperature of 20° C.) for at least 15 minutes. This may be repeated at least 2 times.
3.34 Process 3.33, wherein the process comprises filtering the mixture.
3.35 Any of Process 3.15-3.34, wherein the process comprises rinsing the filter cake with ethanol and water (e.g., a 1:1 mixture of ethanol and water).
3.36 Any of Process 3.34 or 3.35, wherein the process comprises drying the filter cake (e.g., drying the filter cake to a dry content of ≥50% w/w, e.g., drying the filter cake to a dry content of ≥50% w/w and the solid is transferable).
3.37 Any of Process 3.15-3.36, wherein the process comprises stirring (e.g., in a reactor) the trans sodium crocetinate (e.g., the filter cake) with ethanol (e.g., at ambient temperature, e.g., at ambient temperature for at least 15 minutes). For instance, any of Process 3.15-3.36, wherein the process comprises stirring (e.g., in a reactor) the trans sodium crocetinate (e.g., the filter cake) with ethanol (e.g., at an external temperature of 20° C., e.g., at an external temperature of 20° C. for at least 10 minutes).
3.38 Process 3.37, wherein the process comprises filtering the mixture.
3.39 Process 3.38, wherein the process comprises rinsing the filter cake with ethanol.
3.40 Any of Process 3.15-3.39, wherein the process comprises drying the trans sodium crocetinate (e.g., drying the trans sodium crocetinate (e.g., the filter cake) to a dry content of ≥98% w/w).
3.41 Any of Process 3.15-3.40, wherein the process comprises drying (or further drying) the trans sodium crocetinate on a rotary evaporator.
3.42 Any of Process 3 or 3.1-3.41, wherein the trans sodium crocetinate has ≤0.4% w/w ethanol (e.g., as determined by nuclear magnetic resonance (NMR) spectroscopy).
3.43 Any of Process 3 or 3.1-3.42, wherein the trans sodium crocetinate is optionally milled.
3.44 Any of Process 3 or 3.1-3.43, wherein the process comprises seeding (e.g., seeding a solution or slurry) with crystals of the desired form. For instance, any of Process 3 or 3.1-3.43, wherein the process comprises seeding (e.g., seeding a solution or slurry) with Crystalline Form A, e.g., any of Crystalline Form A or A.1 et seq. Or, for instance, any of Process 3 or 3.1-3.43, wherein the process comprises seeding (e.g., seeding a solution or slurry) with Crystalline Form F, e.g., any of Crystalline Form F or F.1 et seq. Or, for instance, any of Process 3 or 3.1-3.43, wherein the process comprises seeding (e.g., seeding a solution or slurry) with Crystalline Form B, e.g., any of Crystalline Form B or B.1 et seq.
3.45 Any of Process 3 or 3.1-3.44, wherein the process comprises isolating trans sodium crocetinate.
3.46 Any of Process 3 or 3.1-3.45, wherein the process comprises isolating trans sodium crocetinate in crystalline form (e.g., in any of the crystalline forms described herein).
3.47 Any of Process 3 or 3.1-3.46, wherein the process comprises isolating any of Crystalline Form A or A.1 et seq.
3.48 Any of Process 3 or 3.1-3.47, wherein the process comprises isolating any of Crystalline Form B or B.1 et seq.
3.49 Any of Process 3 or 3.1-3.48, wherein the process comprises isolating any of Crystalline Form F or F.1 et seq.
3.50 Trans sodium crocetinate made by any of Process 3 et seq.
3.51 Trans sodium crocetinate in crystalline form made by any of Process 3 et seq.
3.52 Any of Crystalline Forms A-G or J vide supra (e.g., any of Crystalline Form A or A.1 et seq. or any of Crystalline Form B or B.1 et seq. or any of Crystalline Form C or C.1 et seq. or any of Crystalline Form D or D.1 et seq. or any of Crystalline Form E or E.1 et seq. or any of Crystalline Form F or F.1 et seq. or any of Crystalline Form G or G.1 et seq. or any of Crystalline Form J or J.1 et seq.), wherein the crystalline form is made by any of Process 3 et seq.
3.53 Any of Process 3 et seq., wherein the process comprises crystallizing trans sodium crocetinate from ethanol, n-butanol, and water. For instance, Process 3 et seq., wherein the process comprises crystallizing trans sodium crocetinate from ethanol, n-butanol, water, and sodium hydroxide. For instance, Process 3 et seq., wherein the process comprises crystallizing trans sodium crocetinate from 6:1 to 1:1 n-butanol:ethanol (e.g., 4:1 n-butanol:ethanol) and optionally with water or optionally with water and sodium hydroxide (e.g., at room temperature or below room temperature). For instance, Process 3 et seq., wherein the process comprises crystallizing trans sodium crocetinate from a mixture of n-butanol, ethanol, and water, wherein the mixture comprises 6:1 to 1:1 n-butanol:ethanol (e.g., 4:1 n-butanol:ethanol) and 30:1 to 10:1 water:ethanol (e.g., 20:1 to 15:1 water:ethanol) and optionally with sodium hydroxide (e.g., at room temperature or below room temperature). For instance, any of Process 3 et seq., wherein trans sodium crocetinate is dissolved in a solution of ethanol, n-butanol, and water, and optionally sodium hydroxide, and the mixture is concentrated to crystallize trans sodium crocetinate.
3.54 Any of Process 3 et seq., wherein the process is as described in any of the synthetic examples herein.

Further provided is a method (Method 1) of treating or controlling a disease or condition characterized by low oxygen (hypoxia) in a patient in need thereof, wherein the method comprises administering to the patient an effective amount (or a pharmaceutical composition comprising an effective amount) of a crystalline form of trans sodium crocetinate (Formula I), e.g., any of Crystalline Forms A-G or J vide supra (e.g., any of Crystalline Form A or A.1 et seq. or any of Crystalline Form B or B.1 et seq. or any of Crystalline Form C or C.1 et seq. or any of Crystalline Form D or D.1 et seq. or any of Crystalline Form E or E.1 et seq. or any of Crystalline Form F or F.1 et seq. or any of Crystalline Form G or G.1 et seq. or any of Crystalline Form J or J.1 et seq.). For instance, Method 1, wherein the disease or condition is described in any of U.S. Pat. Nos. 7,351,844, 7,759,506, 8,030,350, 8,293,804, 8,974,822, and 10,130,689, U.S. Patent Publication Nos. 2019/0083439 and 2021/0244698, and International Publication No. WO 2021/202735.

### BRIEF DESCRIPTION OF THE DRAWINGS

FIG. 1 depicts an XRPD pattern of Crystalline Form A collected with Cu radiation. Vertical axis is Intensity (Counts) and horizontal axis is Diffraction Angle (°2θ).

FIG. 2 depicts an XRPD pattern of Crystalline Form A after dynamic vapor sorption analysis collected with Cu radiation. Vertical axis is Intensity (Counts) and horizontal axis is Diffraction Angle (°2θ).

FIG. 3 depicts a differential scanning calorimetry (DSC) analysis of Crystalline Form A. Vertical axis is Heat Flow (W/g) and horizontal axis is Temperature (° C.).

FIG. 4 depicts a thermogravimetric analysis (TGA) of Crystalline Form A. Vertical axis is Weight (%) and horizontal axis is Temperature (° C.).

FIG. 5 depicts a differential scanning calorimetry (DSC) and thermogravimetric analysis (TGA) overlay for Crystalline Form A. Vertical axis on left is Heat Flow (W/g), vertical axis on right is Weight (%), and horizontal axis is Temperature (° C.).

FIG. 6 depicts a dynamic vapor sorption/desorption isotherm of Crystalline Form A. Vertical axis is % Weight Change and horizontal axis is % Relative Humidity (RH). Solid line is sorption and dotted line is desorption.

FIG. 7 depicts an XRPD pattern of Crystalline Form B collected with Cu radiation. Vertical axis is Intensity (Counts) and horizontal axis is Diffraction Angle (°2θ).

FIG. 8 depicts an XRPD pattern of Crystalline Form B collected with Cu radiation. Vertical axis is Intensity (Counts) and horizontal axis is Diffraction Angle (°2θ).

FIG. 9 depicts an XRPD pattern of Crystalline Form B collected with Cu radiation. Vertical axis is Intensity (Counts) and horizontal axis is Diffraction Angle (°2θ).

FIG. 10 depicts a differential scanning calorimetry (DSC) analysis of Crystalline Form B. Vertical axis is Heat Flow (W/g) and horizontal axis is Temperature (° C.).

FIG. 11 depicts a thermogravimetric analysis (TGA) of Crystalline Form B. Vertical axis is Weight (%) and horizontal axis is Temperature (° C.).

FIG. 12 depicts an XRPD pattern of Crystalline Form A collected with Cu radiation. Vertical axis is Intensity (Counts) and horizontal axis is Diffraction Angle (°2θ).

FIG. 13 depicts an XRPD pattern of Crystalline Form B collected with Cu radiation. Vertical axis is Intensity (Counts) and horizontal axis is Diffraction Angle (°2θ).

FIG. 14 depicts an XRPD pattern of Crystalline Form C collected with Cu radiation. Vertical axis is Intensity (Counts) and horizontal axis is Diffraction Angle (°2θ).

FIG. 15 depicts an XRPD pattern of Crystalline Form C collected with Cu radiation. Vertical axis is Intensity (Counts) and horizontal axis is Diffraction Angle (°2θ).

FIG. 16 depicts a differential scanning calorimetry (DSC) analysis of Crystalline Form C. Vertical axis is Heat Flow (W/g) and horizontal axis is Temperature (° C.).

FIG. 17 depicts a thermogravimetric analysis (TGA) of Crystalline Form C. Vertical axis is Weight (%) and horizontal axis is Temperature (° C.).

FIG. 18 depicts an XRPD pattern of Crystalline Form D collected with Cu radiation. Vertical axis is Intensity (Counts) and horizontal axis is Diffraction Angle (°2θ).

FIG. 19 depicts a differential scanning calorimetry (DSC) analysis of Crystalline Form D. Vertical axis is Heat Flow (W/g) and horizontal axis is Temperature (° C.).

FIG. 20 depicts a thermogravimetric analysis (TGA) of Crystalline Form D. Vertical axis is Weight (%) and horizontal axis is Temperature (° C.).

FIG. 21 depicts an XRPD pattern of Crystalline Form D collected with Cu radiation. Vertical axis is Intensity (Counts) and horizontal axis is Diffraction Angle (°2θ).

FIG. 22 depicts an XRPD pattern of Crystalline Form E collected with Cu radiation. Vertical axis is Intensity (Counts) and horizontal axis is Diffraction Angle (°2θ).

FIG. 23 depicts a differential scanning calorimetry (DSC) analysis of Crystalline Form E. Vertical axis is Heat Flow (W/g) and horizontal axis is Temperature (° C.).

FIG. 24 depicts a thermogravimetric analysis (TGA) of Crystalline Form E. Vertical axis is Weight (%) and horizontal axis is Temperature (° C.).

FIG. 25 depicts an XRPD pattern of Crystalline Form F collected with Cu radiation. Vertical axis is Intensity (Counts) and horizontal axis is Diffraction Angle (°2θ).

FIG. 26 depicts an XRPD pattern of Crystalline Form F collected with Cu radiation. Vertical axis is Intensity (Counts) and horizontal axis is Diffraction Angle (°2θ).

FIG. 27 depicts an XRPD pattern of Crystalline Form F collected with Cu radiation. Vertical axis is Intensity (Counts) and horizontal axis is Diffraction Angle (°2θ).

FIG. 28 depicts a differential scanning calorimetry (DSC) analysis of Crystalline Form F. Vertical axis is Heat Flow (W/g) and horizontal axis is Temperature (° C.).

FIG. 29 depicts a thermogravimetric analysis (TGA) of Crystalline Form F. Vertical axis is Weight (%) and horizontal axis is Temperature (° C.).

FIG. 30 depicts an XRPD pattern of Crystalline Form B collected with Cu radiation. Vertical axis is Intensity (Counts) and horizontal axis is Diffraction Angle (°2θ).

FIG. 31 depicts an XRPD pattern of Crystalline Form B collected with Cu radiation. Vertical axis is Intensity (Counts) and horizontal axis is Diffraction Angle (°2θ).

FIG. 32 depicts an XRPD pattern of Crystalline Form B collected with Cu radiation. Vertical axis is Intensity (Counts) and horizontal axis is Diffraction Angle (°2θ).

FIG. 33 depicts an XRPD pattern of Crystalline Form B collected with Cu radiation. Vertical axis is Intensity (Counts) and horizontal axis is Diffraction Angle (°2θ).

FIG. 34 depicts an XRPD pattern of Crystalline Form B collected with Cu radiation. Vertical axis is Intensity (Counts) and horizontal axis is Diffraction Angle (°2θ).

FIG. 35 depicts an XRPD pattern of Crystalline Form B collected with Cu radiation. Vertical axis is Intensity (Counts) and horizontal axis is Diffraction Angle (°2θ).

FIG. 36 depicts an XRPD pattern of Crystalline Form F collected with Cu radiation. Vertical axis is Intensity (Counts) and horizontal axis is Diffraction Angle (°2θ).

FIG. 37 depicts an XRPD pattern of Crystalline Form D collected with Cu radiation. Vertical axis is Intensity (Counts) and horizontal axis is Diffraction Angle (°2θ).

FIG. 38 depicts an XRPD pattern of the product from Example 20 collected with Cu radiation. Vertical axis is Intensity (Counts) and horizontal axis is Diffraction Angle (°2θ).

FIG. 39 depicts an XRPD pattern of Crystalline Form J collected with Cu radiation. Vertical axis is Intensity (Counts) and horizontal axis is Diffraction Angle (°2θ).

FIG. 40 depicts an XRPD pattern of Crystalline Form G collected with Cu radiation. Vertical axis is Intensity (Counts) and horizontal axis is Diffraction Angle (°2θ).

FIG. 41 depicts an XRPD pattern of Crystalline Form G collected with Cu radiation. Vertical axis is Intensity (Counts) and horizontal axis is Diffraction Angle (°2θ). The XRPD is taken using a Miniflex goniometer, operating with Cu/30 kV/15 mA, using a Kb filter, in continuous scan mode, with a sampling width of 0.02°, a scan range of 2°-40°, and a scan speed of 0.5°/minute.

FIG. 42 depicts a differential scanning calorimetry (DSC) and thermogravimetric analysis (TGA) overlay for Crystalline Form J. Vertical axis on left is Heat Flow (W/g), vertical axis on right is Weight (%), and horizontal axis is Temperature (° C.).

FIG. 43 depicts a dynamic vapor sorption/desorption isotherm of the product from Example 20. Vertical axis is % Weight Change and horizontal axis is % Relative Humidity (RH).

FIG. 44 depicts an XRPD pattern of the product from Example 20 after dynamic vapor sorption analysis collected with Cu radiation. Vertical axis is Intensity (Counts) and horizontal axis is Diffraction Angle (°2θ).

FIG. 45 depicts an XRPD pattern of the product from Example 21 collected with Cu radiation. Vertical axis is Intensity (Counts) and horizontal axis is Diffraction Angle (°2θ).

FIG. 46 depicts an XRPD pattern discussed in Example 25 collected with Cu radiation. Vertical axis is Intensity (Counts) and horizontal axis is Diffraction Angle (°2θ).

FIG. 47 depicts an XRPD pattern discussed in Example 25 collected with Cu radiation. Vertical axis is Intensity (Counts) and horizontal axis is Diffraction Angle (°2θ).

FIG. 48 depicts an XRPD pattern of Crystalline Form G collected with Cu radiation. Vertical axis is Intensity (Counts) and horizontal axis is Diffraction Angle (°2θ).

FIG. 49 depicts an XRPD pattern of Crystalline Form E collected with Cu radiation. Vertical axis is Intensity (Counts) and horizontal axis is Diffraction Angle (°2θ).

FIG. 50 depicts an XRPD pattern of a pharmaceutical composition collected with Cu radiation. Vertical axis is Intensity (Counts) and horizontal axis is Diffraction Angle (°2θ).

### DETAILED DESCRIPTION

Trans sodium crocetinate (TSC) is shown as Formula I below

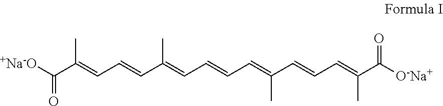

The crystallinity, morphology, and properties of the crystals described herein, e.g., any of Crystalline Forms A-G or J, e.g., any of Crystalline Form A or A.1 et seq., Crystalline Form B or B.1 et seq., Crystalline Form C or C.1 et seq., Crystalline Form D or D.1 et seq., Crystalline Form E or E.1 et seq., Crystalline Form F or F.1 et seq., Crystalline Form G or G.1 et seq., and any of Crystalline Form J or J.1 et seq., may be determined by a number of methods, including, but not limited to single crystal X-ray diffraction, X-ray powder diffraction, polarizing optical microscopy, thermal microscopy, differential scanning calorimetry (DSC), thermogravimetric analysis (TGA), dynamic (water) vapor sorption, infrared absorption spectroscopy, and Raman spectroscopy.

Processes disclosed herein may provide improved yield, purity, and/or work-up compared to other processes.

Uncontrolled precipitation of a product can result in unpredictable qualities of the final product, including which, if any, crystalline form may result. Identifying if polymorphs of a compound exist and their properties and how they interact is useful to understand how to control a process to ensure predictable qualities of the final product, including its crystalline form. Processes disclosed herein (e.g., with n-butanol and ethanol and sodium base) allow for dissolution of trans sodium crocetinate in a useful concentration that may beneficial for controlled crystallization of trans sodium crocetinate from solution.

It is to be understood that an XRPD pattern of a given sample may vary (standard deviation) depending on the instrument used, the time and temperature of the sample when measured, and standard experimental errors. Therefore, the 2-theta values (°2θ), d-spacing values, heights and relative intensity of the peaks will have an acceptable level of deviation. For example, the values may have an acceptable deviation of e.g., 20%, 15%, 10%, 5%, 3%, 2%, or 1%. In a particular embodiment, the 2-theta (°) values or the d-spacing (Å) values of the XRPD patterns of the crystalline forms described herein may have an acceptable deviation of ±0.2° and/or ±0.2 Å. Further, the XRPD patterns of the crystalline forms described herein may be identified by characteristic and/or representative peak(s) as recognized by one skilled in the art.

Peak position variabilities in the tables listing 2θ values vide supra (Tables A-G and J and Tables AA-GG) are given to within ±0.2° 2θ. Third party measurements on independently prepared samples on different instruments may lead to variability which is greater than ±0.2° 2θ.

It is also to be understood that the differential scanning calorimetry and thermogravimetric analysis thermograms of a given sample may vary (standard deviation) depending on the instrument used, the time and temperature of the sample when measured, and standard experimental errors. The temperature value itself may deviate by ±10° C., e.g., ±5° C., e.g., ±3° C., of the reference temperature.

If multiple diffraction patterns are available, then assessments of particle statistics (PS) and/or preferred orientation (PO) are possible. Preferred orientation occurs when there is a tendency for crystals to be oriented more in one way than other. Reproducibility among XRPD patterns from multiple samples analyzed on a single diffractometer indicates that the particle statistics are adequate. Consistency of relative intensity among XRPD patterns from multiple diffractometers indicates good orientation statistics. Alternatively, the observed XRPD pattern may be compared with a calculated XRPD pattern based upon a single crystal structure, if available. Two-dimensional scattering patterns using area detectors can also be used to evaluate PS/PO. If the effects of both PS and PO are determined to be negligible, then the XRPD pattern is representative of the powder average intensity for the sample and prominent peaks may be identified as “representative peaks.” In general, the more data collected to determine representative peaks, the more confident one can be of the classification of those peaks.

“Characteristic peaks,” to the extent they exist, are a subset of representative peaks and are used to differentiate one crystalline polymorph from another crystalline polymorph (polymorphs being crystalline forms having the same chemical composition). Characteristic peaks are determined by evaluating which representative peaks, if any, are present in one crystalline polymorph of a compound against all other known crystalline polymorphs of that compound to within ±0.2 °2θ. Not all crystalline polymorphs of a compound necessarily have at least one characteristic peak.

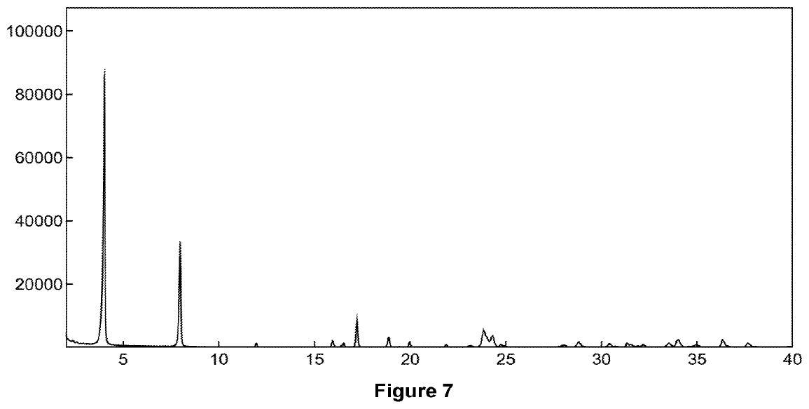

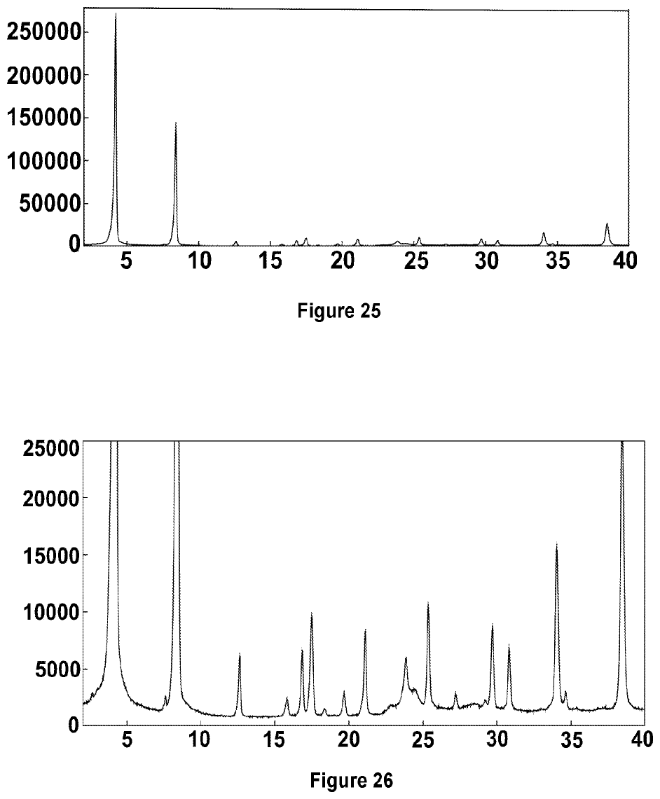

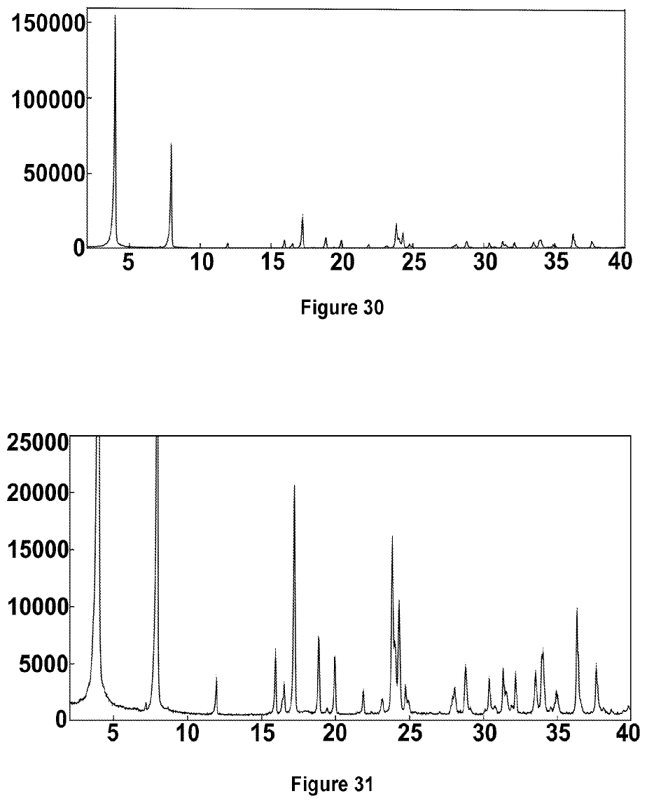

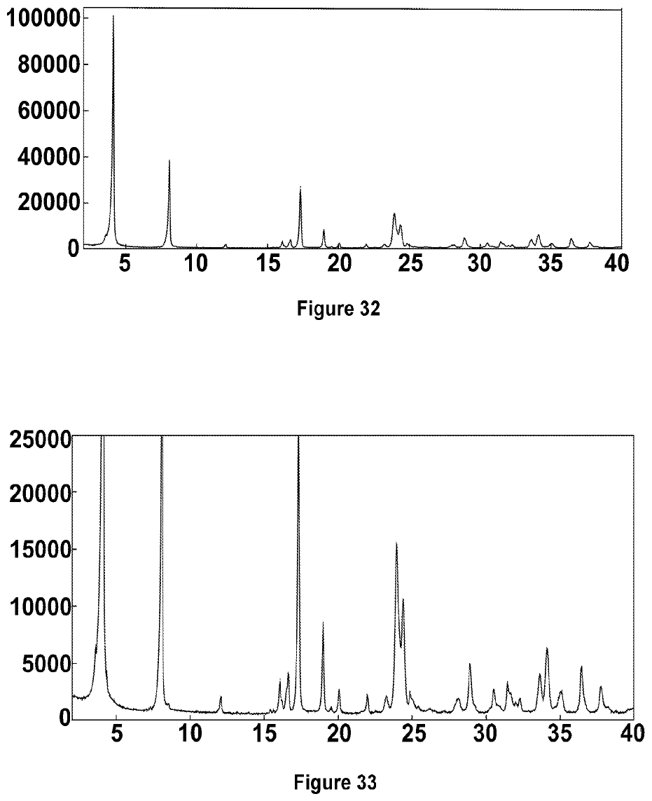

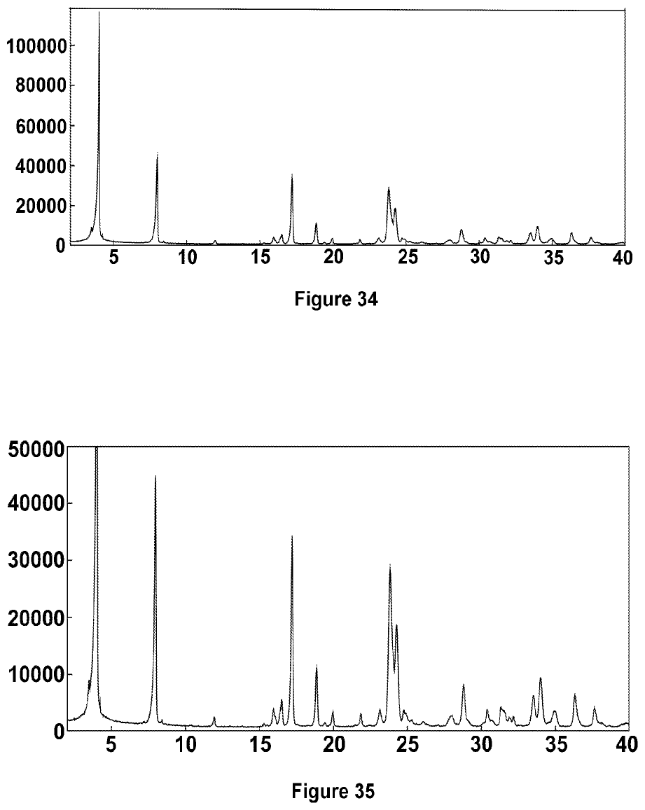

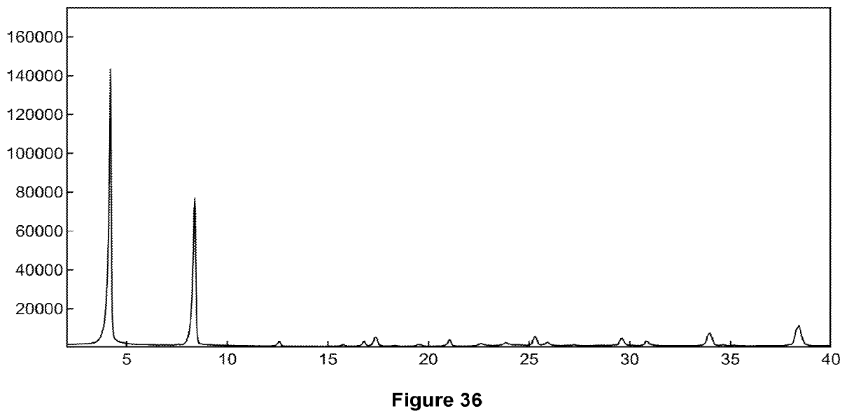

Examination of crystalline materials disclosed herein by microscopy show fine powders, however, XRPD patterns of crystalline forms herein may show variability in peak intensity. For instance, compare FIG. 7, FIG. 30, FIG. 32, and FIG. 34, which are XRPD patterns of Crystalline Form B. In addition, compare FIG. 25 and FIG. 36, which are XRPD patterns of Crystalline Form F, and compare XRPD patterns of Crystalline Form D.

As used herein, “XRPD” means X-ray powder diffraction. XRPDs described herein may be obtained with Cu—Kα radiation.

As used herein, “patient” includes human and non-human. In one embodiment, the patient is a human. In another embodiment, the patient is a non-human.

As used herein, the term “hydrate” refers to a crystal containing either stoichiometric or nonstoichiometric amounts of water incorporated within the crystal structure.

As used herein, the term “solvate” refers to a crystal containing either stoichiometric or nonstoichiometric amounts of a solvent incorporated within the crystal structure.

As used herein, the term “non-solvate” refers to a crystal that is free or substantially free of solvent molecules within the crystal structure.

As used herein, the term “non-hydrate” refers to a crystal that is free or substantially free of water molecules within the crystal structure.

As used herein, the term “amorphous” refers to solids of disordered arrangements of molecules and do not possess a distinguishable crystal lattice.

As used herein, “anti-solvent” means a solvent in which trans sodium crocetinate (Formula I) has low solubility or is insoluble. For instance, an anti-solvent includes a solvent in which Formula I has a solubility of less than 35 mg/ml, e.g., a solubility of 10-30 mg/ml, e.g., a solubility of 1-10 mg/ml, e.g., a solubility of less than 1 mg/ml.

The wavelength used to calculate the d-spacing (Å) values herein is 1.5406 Å. Variability associated with d-spacing values may be calculated from the USP recommendation.

A formulated drug product containing a crystalline form of TSC (e.g., any crystalline form described herein) may be analyzed by XRPD to identify the crystalline form in the formulated drug product. This can be achieved by deconvoluting the XRPD data obtained from the formulated drug product. This can be achieved by subtracting the known excipient signals from the XRPD data of the formulated drug product. In addition, various chemometrics techniques can be used to identify the crystalline form in the formulated drug product, for example principal component analysis. Such analysis may be required to identify XRPD peaks belonging to the crystalline form as they may overlap with XRPD peaks of some of the excipients used in the formulation.

### EXAMPLES

### Abbreviations

eq=equivalents
h=hour(s)
ML=mother liquor
RT=room temperature
Ta/Ti=External Temperature/Internal Temperature
Vol (vol)=Volumes

### Example 1—General Procedures

### Example 1A—X-Ray Powder Diffraction (XRPD)

The Rigaku Smart-Lab X-ray diffraction system is configured for reflection Bragg-Brentano geometry using a line source X-ray beam. The x-ray source is a Cu Long Fine Focus tube that is operated at 40 kV and 44 ma. The x-ray wavelength is 1.5406 Å. That source provides an incident beam profile at the sample that changes from a narrow line at high angles to a broad rectangle at low angles. Beam conditioning slits are used on the line X-ray source to ensure that the maximum beam size is less than 10 mm both along the line and normal to the line. The Bragg-Brentano geometry is a para-focusing geometry controlled by passive divergence and receiving slits with the sample itself acting as the focusing component for the optics. The inherent resolution of Bragg-Brentano geometry is governed in part by the diffractometer radius and the width of the receiving slit used. Typically, the Rigaku Smart-Lab is operated to give peak widths of 0.1 °2θ or less. The axial divergence of the X-ray beam is controlled by 5.0-degree Soller slits in both the incident and diffracted beam paths.

Powder samples are prepared in a low background Si holder using light manual pressure to keep the sample surfaces flat and level with the reference surface of the sample holder. Each sample is analyzed from 2 to 40 °2θ using a continuous scan of 6 °2θ per minute with an effective step size of 0.02 °2θ.

It is possible to analyze the materials using a similar XRPD system equipped with a Cu x-ray source, using a Bragg-Brentano geometry and step size of 0.02° 2θ. It is possible to obtain similar quality XRPD diffractograms by varying incident/receiving slits, step sizes, and scan speed.

### Example 1B—Differential Scanning Calorimetry (DSC)

DSC analyses are carried out using a TA Instruments Q2500 Discovery Series instrument. The instrument temperature calibration is performed using indium. The DSC cell is kept under a nitrogen purge of ˜50 mL per minute during each analysis. The sample (about 1-2 mg) is placed in a standard, crimped, aluminum pan and is heated from approximately 25° C. to 350° C. at a rate of 10° C. per minute. The type of pan, preparation of the pan for analysis, and heating rate may impact the outcome of the analysis. Nitrogen purge rate can be varied as appropriate for the specific instrument specifications.

### Example 1C—Thermogravimetric (TG) Analysis

The TG analysis is carried out using a TA Instruments Q5500 Discovery Series instrument or a TA Instruments Q50 instrument. The instrument balance is calibrated using class M weights and the temperature calibration is performed using alumel. The nitrogen purge is ˜40 mL per minute at the balance and ˜60 mL per minute at the furnace. Each sample (about 2-5 mg) is placed into a pre-tared platinum pan and heated from approximately 25° C. to 350° C. at a rate of 10° C. per minute. The heating rate may impact the outcome of the analysis results. Nitrogen purge rate can be varied as appropriate for the specific instrument specifications.

### Example 1D—Dynamic Vapor Sorption (DVS) Analysis

DVS analysis is carried out using a TA Instruments Q5000 Dynamic Vapor Sorption analyzer. The instrument is calibrated with standard weights and a sodium bromide standard for humidity. Approximately 10-25 mg (e.g., 20 mg) or 5-15 mg of sample is loaded into a metal-coated quartz pan for analysis. The sample is analyzed at 25° C. with a maximum equilibration time of one hour in 10% relative humidity (RH) steps from 5 to 95% RH (adsorption cycle) and from 95 to 5% RH (desorption cycle). The movement from one step to the next occurred either after satisfying the equilibrium criterion of 0.01% weight change or, if the equilibrium criterion is not met, after one hour. The percent weight change values are calculated using Microsoft Excel®. The temperature for the DVS analysis may impact the outcome of the results.

### Example 1E—Raman Spectroscopy

Fourier transform (FT) Raman spectra are acquired on a Nicolet model 6700 spectrometer interfaced to a Nexus Raman accessory module. This instrument is configured with a Nd:YAG laser operating at 1024 nm, a CaF₂ beam-splitter, and a indium gallium arsenide detector. OMNIC 9.7.46 software is used for control of data acquisition and processing of the spectra. Samples are packed into a 3-inch glass NMR tube for analysis. The FT-Raman spectrum is collected with 256 signal-averaged scans at a resolution of 4 cm⁻¹ over the spectral range 3700-100 cm⁻¹.

### Example 1F—Nuclear Magnetic Resonance (NMR) Spectroscopy

The ¹H NMR spectra are acquired on a Bruker Avance II 400 spectrometer. Samples are prepared by dissolving material in DMSO-d₆. The solutions are filtered and placed into individual 5-mm NMR tubes for subsequent spectral acquisition. The temperature controlled (295K)¹H NMR spectra acquired on the Avance II 400 utilize a 5-mm cryoprobe operating at an observing frequency of 400.18 MHz.

### Example 1G—Cooling Experiment

Sample preparation is carried out in a nitrogen glove box. 25.6 mg of TSC from Example 2 is placed in a 1-dram glass vial with 1.0 mL of 50:50 THF:water (v/v) and stirred magnetically on a 60° C. hot plate. Solids remain after approximately 2 hours, so an additional 1.5 mL of solvent is added. A clear orange solution results. The heat is turned off and the sample is allowed to cool to room temperature. No solid is observed after 1 day at room temperature, so the sample is moved to a refrigerator (˜2° C.). Solid is observed after 20 days in the refrigerator. The mother liquor is decanted and the solid is allowed to air dry in a nitrogen glove box at room temperature prior to XRPD analysis.

### Example 1H—Slurry Experiment

Sample preparation is carried out in a nitrogen glove box. 26.1 mg of TSC from Example 2 is placed in a 1-dram glass vial with 0.45 mL of 50:50 acetone:water (v/v). The slurry is stirred magnetically at room temperature for 10 days. The sample is centrifuged and the mother liquor is decanted. The resulting solid is allowed to air dry in a nitrogen glove box at room temperature prior to XRPD analysis.

### Example 1I—Grinding Experiment

Sample preparation is carried out in a nitrogen glove box. 24.7 mg of TSC from Example 2 is placed in a PEEK grinding cup. The internal volume of the cup is approximately 2 mL. 10 μL of acetone and 10 μL of water are added to the cup along with a stainless-steel ball. The grinding cup is placed on a Retsch Mill and milled at 100% power for 30 minutes. The resulting solid is allowed to air dry under nitrogen prior to XRPD analysis.

### Example 1J—Lyophilization Experiment

Sample preparation is carried out in a nitrogen glove box. 26.8 mg of TSC from Example 2 is placed in a glass vial with 10 mL of water. The vial is sonicated to dissolve the solid. The solution is transferred to a 50 mL round bottom flask which is rotated in a dry-ice/acetone bath to freeze the solution to the sides of the flask wall. The flask is then placed on a LabConco FreeZone 1 lyophilizer. Solid is present the next day. The solid is transferred to a 2-dram glass vial and 6 mL of methanol is added. The slurry is stirred magnetically at room temperature for 5 days. The sample is centrifuged and the mother liquor is decanted. The resulting solid is allowed to air dry in a nitrogen glove box at room temperature prior to XRPD analysis.

### Example 1K—Antisolvent Precipitation

Sample preparation is carried out in a nitrogen glove box. 25.4 mg of TSC from Example 2 is placed in a 20 mL glass vial with 7 mL of water. The vial is sonicated to dissolve the solid. Antisolvent is then added, 13 mL of dioxane. The solution becomes hazy. The sample is capped and stirred magnetically at room temperature for 5 days. The solution remains hazy so it is transferred to a 40 mL glass vial and additional dioxane is added (15 mL). The hazy solution is stirred magnetically at room temperature for 6 days, during which time additional solid crystallized. The solid is recovered by vacuum filtration and analyzed by XRPD.

### Example 1L—Evaporation

Sample preparation is carried out in a nitrogen glove box. 25.1 mg of TSC from Example 2 is placed in a 20 mL glass vial with 5 mL of 20:80 acetone:water (v/v). The vial is sonicated to dissolve the solid. The vial is left uncapped and the solvent allowed to evaporate at room temperature. The resulting solid is analyzed by XRPD.

### Example 1M—Competitive Slurry

Sample preparation is carried out in a nitrogen glove box. Saturated solutions of TSC from Example 2 are prepared at a given temperature. The volume of each solution is about 1.5 mL and it is deemed saturated if excess solid remains after stirring for 2 hours. The sample is then centrifuged and the mother liquor is transferred to a vial containing equal amounts of each polymorph, about 10 mg each. The vials are capped and sealed with parafilm, and the slurries are then stirred magnetically at a given temperature for 5 days. Each sample is then centrifuged, the mother liquor decanted, and the wet solid immediately analyzed by XRPD.

### Syntheses

If not commercially available, starting materials may be made by procedures which are selected from the chemical art using techniques which are similar or analogous to the synthesis of known compounds.

### Example 2—Trans Sodium Crocetinate (Form A)

### Step 1

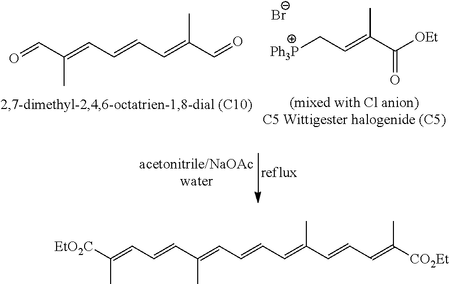

### General Process Description

### Reaction

Amounts are L/kg related to 2,7-dimethyl-2,4,6-octatrien-1,8-dial (C10) (3 kg). Charge reactor with 6 equivalents of USP Grade NaOAc (9 kg). Dissolve 2,7-dimethyl-2,4,6-octatrien-1,8-dial (C10) and C5 Wittigester halogenide (C5) (20.4 kg, 2.38 equivalents) in 100 L/kg acetonitrile (in portions if necessary) in abrasion resistant glass. Fine filtration of the solution from abrasion resistant glass into the reactor. Distillation to 19 L/kg (8 L/kg acetonitrile residual content) at 121-200 mbar. Add 0.1 equivalent of DI or purified water. Ta: 85° C. for 15-72 hours.

### Isomerization

Add 10 L/kg of DI or purified water. Stir for at least 12 hours. Cool to Ti: 60° C.

### Work Up

Add 4 L/kg of ethanol. Add 2 L/kg of DI or purified water. Cool to Ti: 20° C. Filter and wash 2 times with ethanol:water 1:1 (by volume), 4 L/kg for each wash and then dry.

### Detailed Process Description

Charge reaction vessel with 9 kg (±5%) of NaOAc.

Charge agitator tank with 10.2 kg (±3%) of C5 Wittigester halogenide (C5) and 1.5 kg (±3%) of 2,7-dimethyl-2,4,6-octatrien-1,8 dial (C10). Charge agitator tank with 150 L or 119 kg (±10%) of acetonitrile. Stir C5, C10, and acetonitrile suspension until a solution is obtained. Transfer solution to feed tank via inline filter (also during distillation). Concentrate at Ta: 80° C. and 121-200 mbar to about 276 L of distillate. During first 20-30 L, reaction vessel is filled with bubbles/foam. Later on boiling is better without large foam.

Charge agitator tank with 10.2 kg (+3%) of C5 Wittigester halogenide (C5) and 1.5 kg (±3%) of 2,7-dimethyl-2,4,6-octatrien-1,8 dial (C10). Charge agitator tank with 150 L or 119 kg (±10%) of acetonitrile. Stir C5, C10, and acetonitrile suspension until a solution is obtained. Transfer solution to feed tank via inline filter (also during distillation). Concentrate at Ta: 80° C. and 121-200 mbar to about 276 L of distillate.

Charge 0.033 L (±10%) of deionized (or purified water). Heat mixture to Ti: 80° C. Stir mixture at Ta: 85° C. for 15-72 hours.

Charge 30 L (±10%) of deionized (or purified water). Stir mixture at Ti: 80° C. for at least 12 hours.

Cool mixture to Ti: 60° C. over at least 20 minutes.

Separate phases. Charge 12.0 L (±10%) of ethanol. Charge 6.0 L (±10%) of deionized (or purified water). Cool mixture to Ti: 20° C. over at least 40 minutes. Stir mixture at Ti: 20° C. for at least 60 minutes. Transfer suspension to filter and remove mother liquor.

Charge 6.0 L (±10%) of deionized (or purified water). Charge 6.0 L (±10%) of ethanol. Stir mixture at Ti: 20° C. for at least 5 minutes. Transfer suspension to filter and remove mother liquor.

Charge 6.0 L (±10%) of deionized (or purified water). Charge 6.0 L (±10%) of ethanol. Stir mixture at Ti: 20° C. for at least 5 minutes. Transfer suspension to filter and remove mother liquor.

Dry on filter for at least 48 hours at p: 500-10 mbar until dry content ≥98% w/w. 4.8 kg obtained.

### Step 2

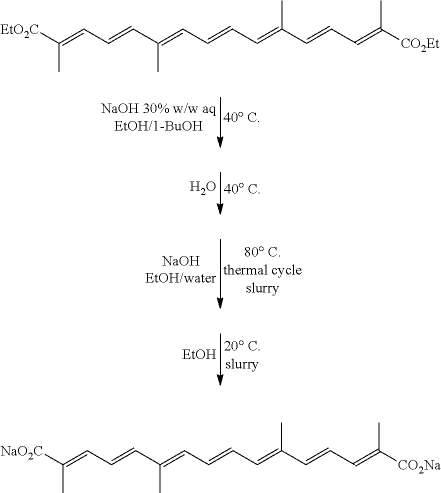

### General Process Description

### Reaction

Amounts are L/kg related to the bis-ethyl crocetinate (C20) made in Step 1. 6.1 kg of bis-ethyl crocetinate (C20) made in Step 1 is charged into the reactor. NaOH 30% 2 L/kg, 1-BuOH 10 L/kg, and EtOH 10 L/kg are filtered inline and loaded into the reactor. Stir at Ti: 40° C. for at least 12 hours. Add 20 L/kg of purified water. Stir at Ti: 40° C. for at least 12 hours. Remove 20 L/kg of distillate at Ta: 50° C. and 70-200 mbar. Cool to Ti: 20° C. over at least 30 minutes. Stir for at least 30 minutes at Ti: 20° C. Filter and rinse reactor with mother liquor. Flush reactor and filter cake with mixture of 2 L/kg inline filtered EtOH and 2 L/kg of water. Dry until dry content is ≥50% and transferable.

### Slurry Washing (Portions are Combined if Reaction is Done Portion Wise)

Charge wet cake(s) into (cleaned) reactor. NaOH 30% 0.08 L/kg, purified water 4 L/kg, and EtOH 4 L/kg are filtered inline and loaded into reactor. Heat up to Ti: 80° C. and stir for at least 1 hour. Cool to Ti: 20° C. over at least 120 minutes. Stir for at least 60 minutes at Ti: 20° C. Filter and rinse reactor with mother liquor. Dry until dry content is ≥50% and transferable.

### Slurry Drying

Invite wet cake. Filter EtOH 10 L/kg inline and load into reactor. At Ta: 20° C., stir for at least 1 hour. Filter and rinse reactor with mother liquor. Rinse reactor and filter cake with inline filtered EtOH 4 L/kg. Dry Nutsche until dry content ≥98%. Drying Rotary evaporator until dry content ≥99.5%. Sieve grind and mix (if not homogenous powder).

### Detailed Process Description

First portion: 2.4 kg (±3%) of bis-ethyl crocetinate (C20) made in Step 1 is charged to the reactor. 23.6 L or 19.1 kg (±10%) of 1-butanol is charged to the reactor. 4.7 or 6.3 kg (±10%) of 30% NaOH is charged to the reactor. 23.6 L or 18.6 kg (±10%) of ethanol is charged to the reactor. Warm the suspension to Ti: 40° C. Stir mixture at Ti: 40° C. for 24-72 hours. Charge reactor with 47.3 L or 47.3 kg (±10%) of purified water. Stir mixture at Ti: 40° C. for at least 12 hours. Concentrate at Ta: 50° C. at 70-200 mbar (43.3 L of distillate±10%). Cool to Ti: 20° C. over at least 30 minutes. Stir mixture at Ti: 20° C. for at least 30 minutes. Transfer suspension to filter and remove mother liquor. Use mother liquor to rinse reactor. Charge 4.7 L or 4.7 kg (±10%) of deionized or purified water. Charge 4.7 L or 3.7 kg (±10%) of ethanol. Stir mixture at Ti: 20° C. for at least 1 minute. Transfer suspension to filter and remove liquor. Dry on filter for at least 5 hours at p: 500-10 mbar (e.g., 200-20 mbar) until dry content ≥50% w/w and transferable. Unload portion (2.8 kg, 2,049 g dry content corrected).

Second portion: 2.4 kg (±3%) of bis-ethyl crocetinate (C20) made in Step 1 is charged to the reactor. 23.6 L or 19.1 kg (±10%) of 1-butanol is charged to the reactor. 4.7 or 6.3 kg (±10%) of 30% NaOH is charged to the reactor. 23.6 L or 18.6 kg (+10%) of ethanol is charged to the reactor. Warm the suspension to Ti: 40° C. Stir mixture at Ti: 40° C. for 24-72 hours. Charge reactor with 47.3 L or 47.3 kg (+10%) of purified water. Stir mixture at Ti: 40° C. for at least 12 hours. Concentrate at Ta: 50° C. at 70-200 mbar (43.3 L of distillate±10%). Cool to Ti: 20° C. over at least 30 minutes. Stir mixture at Ti: 20° C. for at least 30 minutes. Transfer suspension to filter and remove mother liquor. Use mother liquor to rinse reactor. Charge 4.7 L or 4.7 kg (±10%) of deionized or purified water. Charge 4.7 L or 3.7 kg (±10%) of ethanol. Stir mixture at Ti: 20° C. for at least 1 minute. Transfer suspension to filter and remove liquor. Dry on filter for at least 5 hours at p: 500-10 mbar (e.g., 200-20 mbar) until dry content ≥50% w/w and transferable. Unload portion (2.7 kg, 2,294 g dry content corrected).

Charge product from first and portions to reactor (5.4 kg, uncorrected). Charge 18.9 L or 18.9 kg (±10%) of deionized or purified water to the reactor. Charge 0.04 L or 0.05 kg (10%) of 300% NaOH to the reactor. Charge 18.9 L or 14.9 kg (10%) of ethanol to the reactor. Warm the suspension to Ti: 80° C. Stir mixture at Ti: 80° C. for 60 minutes. Cool to Ti: 20° C. over at least 120 minutes. Stir mixture at Ti: 20° C. for at least 60 minutes. Transfer suspension to filter and remove mother liquor. Use mother liquor to rinse reactor. Dry on filter for at least 5 hours at p: 200-20 mbar until dry content ≥50% w/w and transferable.

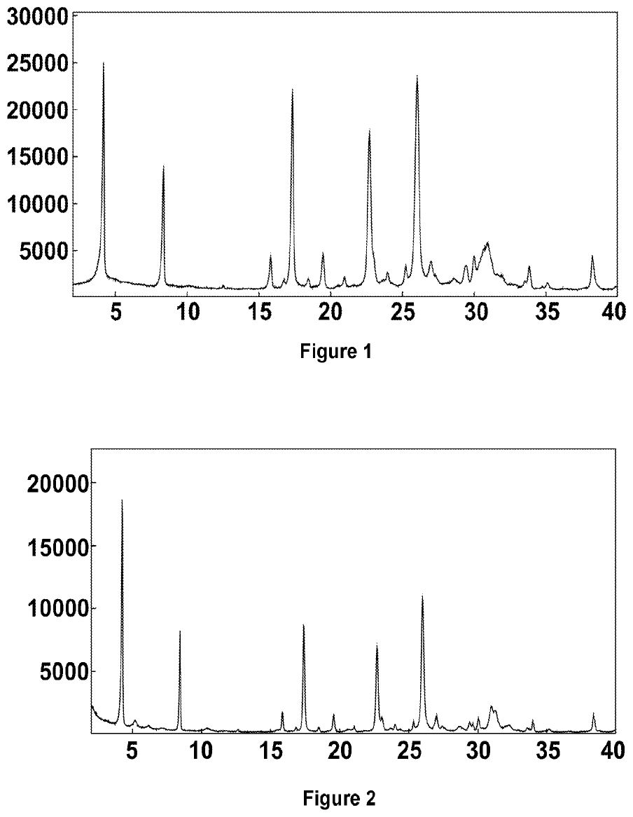

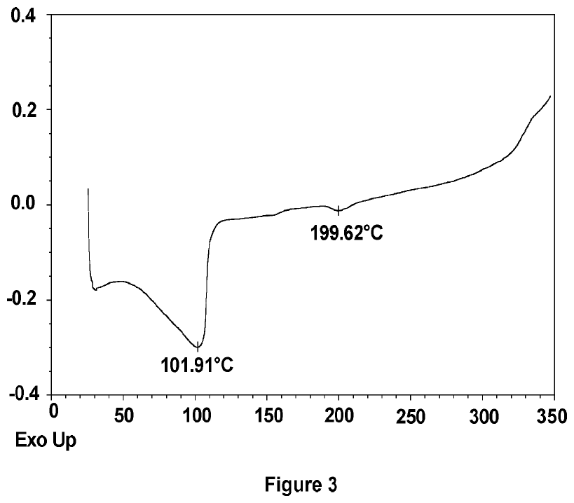

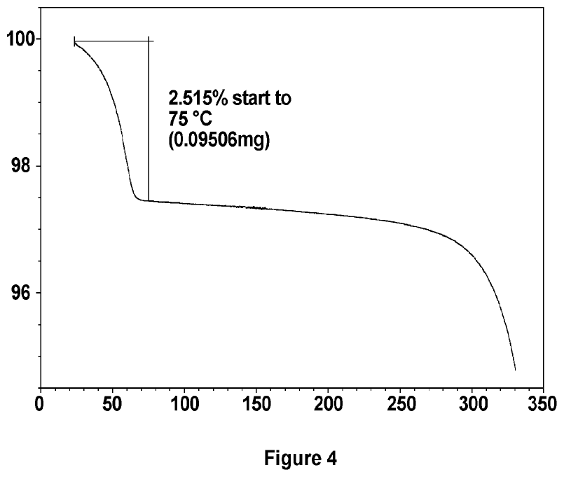

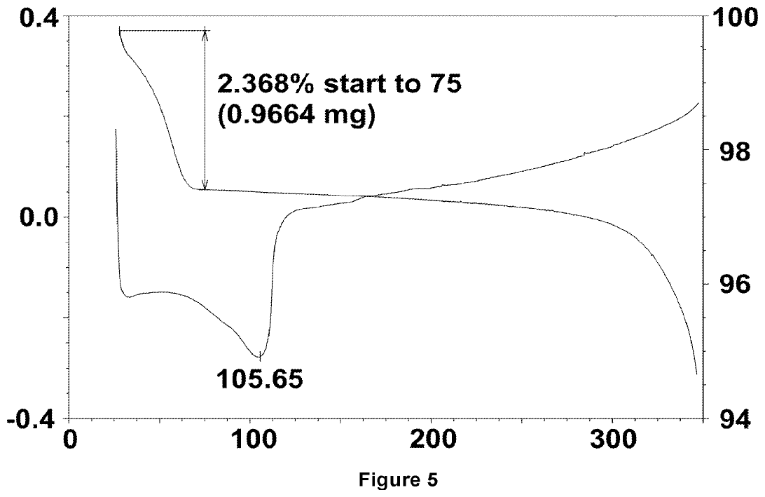

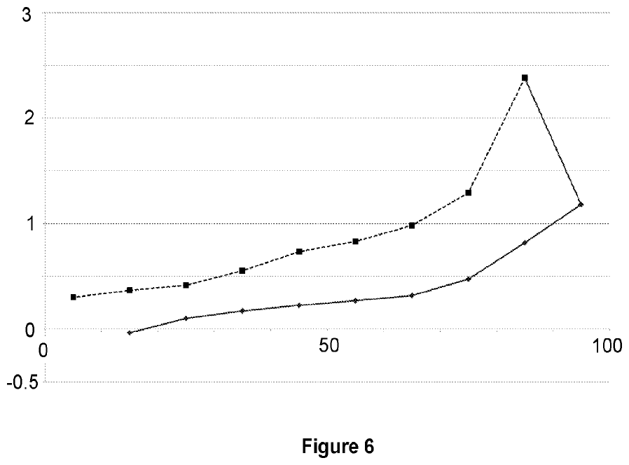

Unload and transfer into reactor. Charge 47.2 L or 37.2 kg (±10%) of ethanol into the reactor. Stir mixture at Ti: 20° C. for at least 60 minutes. Transfer suspension to filter and remove mother liquor. Use mother liquor to rinse reactor. Charge 18.9 L or 14.9 kg (10%) of ethanol into the reactor. Stir mixture at Ti: 20° C. for at least 1 minute. Transfer suspension to filter and remove mother liquor. Dry on filter for at least 5 hours at p: 200-20 mbar until dry content ≥98% w/w. Unload and transfer into rotary evaporator flask. Dry for at least 24 hours at Ta: 55° C., p: 200-10 mbar until dry content ≥99.5% w/w. Decision for sieve milling based on aspect. Solid mixing for at least 1 hour. Yield: 4 kg of trans sodium crocetinate. HPLC: 99.5% w/w. Karl-Fischer: 0.92% w/w. Na content by FAAS: 13.0%. An XRPD of the product is shown in FIG. 1 (refer to Example 1 for XRPD details). An XRPD of the material after a dynamic vapor sorption analysis (refer to Example 1 for details) is in FIG. 2. The material after the DVS experiment remains unchanged by XRPD. FIGS. 3 and 4 depicts a differential scanning calorimetry (DSC) and thermogravimetric analysis (TGA) overlay for the product (refer to Example 1 for experimental details). FIG. 6 depicts a dynamic vapor sorption/desorption isotherm (refer to Example 1 for experimental details) of the product. Crystalline Form A exhibits a maximum water absorption of about 2.5%.

### Example 3—Trans Sodium Crocetinate (Form A)

### Step 1

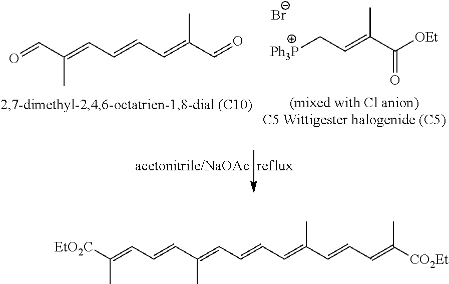

### Materials

| Compound | Mass or Volume | |
|---|---|---|
| C10 | 7 | kg |
| C5 | 47.8 | kg |
| Acetonitrile | 350 | L |
| Sodium acetate (USP grade) | 21 | kg |
| Deionized water | 126 | L |
| Sodium chloride solution (23.1% w/w) | 56 | L |
| Ethanol | 56 | L |

350 L of acetonitrile are charged into a stainless steel reactor and heated to 40° C. Due to limited solubility of raw materials, the next step is performed sequentially in two portions. For the first portion, 3.5 kg of C10 and 23.9 of C5 are charged to a stirred mobile abrasion resistant glass tank. The tank is filled to the 190 L mark with warm acetonitrile, dissolving the two raw materials with only traces of insolubles remaining after 1 h. The mixture is transferred through a 0.2/0.45 m in-line filter into an enamel reactor and concentrated at Tj: 80° C. and reduced pressure (p: 179-196 mbar) until 60 L distillate is collected. For the second portion, 3.5 kg C10 and 23.9 kg C5 are charged to the tank. The tank is filled to the 190 L mark with warm acetonitrile, dissolving the two raw materials with only traces of insolubles remaining after 37 minutes. The mixture is transferred through the 0.2/0.45 m in-line filter into the enamel reactor and concentrated at Tj: 80° C. and reduced pressure (p: 190-226 mbar) until a total of 280 L distillate is collected. 21 kg USP grade sodium acetate is added to the concentrate and the mixture stirred at Tj: 85° C. over the weekend for 67 h (unattended). The orange suspension is then cooled to Ti: 40° C. for sampling. The mixture is then cooled to Ti: 20° C. and stirred for 19 hours. The mixture is stirred for another 24 hours. 70 L deionized water is added and the mixture heated to Ti: 30° C. with maximum stirring. The phases are separated over 1.5 h (OP: dark red suspension, AP: ca. 79 L, yellow, pH 6-7). 56 L aqueous sodium chloride 7.4% w/w is added to the red suspension. The mixture is heated to Ti: 40° C. with maximum stirring. The phases are separated over 1 h (OP: dark red suspension, AP: ca. 68 L, clear yellow, pH 4-5). The red suspension is cooled to Ti: 20° C. and stirred for 1 h before the start of the filtration. The filtration is run using the available 1200 mbar absolute pressure compatible with the glass equipment. As the filtration is sluggish, it is modified from a standard pressure-only filtration by addition of a suction flask to reduce the backpressure of the filter and takes 4.5 days to complete. 14 L deionized water and 14 L inline-filtered ethanol are charged into the reactor and filtered off over the product filter cake over 8 h using the 1250 mbar pressure and the suction flask. 14 L deionized water and 14 L inline-filtered ethanol are charged into the reactor and filtered off over the product filter cake over 7 h using the 1250 mbar pressure and the suction flask.

14 L deionized water and 14 L inline-filtered ethanol are charged into the reactor and part of the wash solution loaded onto the filter. The filter cake is manually slurried and filtered overnight using the 1250 mbar pressure and the suction flask together with the remaining wash solution from the reactor.

14 L deionized water and 14 L inline-filtered ethanol are charged into the reactor and part of the wash solution loaded onto the filter. The filter cake is manually slurried and filtered overnight using the 1250 mbar pressure and the suction flask together with the remaining wash solution from the reactor.

The filter cake is dried at reduced pressure under nitrogen flow for 6 days.

The filter cake is homogenized and drying continued for 7 h.

The filter cake is homogenized and drying continued overnight for 16 h.

The damp filter cake (12.9 kg) is transferred from the filter into a 100 L round bottom flask which is mounted on a rotary evaporator fitted with a sintered glass frit as a protective barrier. The material is dried at Tj: 60° C. at reduced pressure (p: 28-77 mbar) for 5 h.

Drying is continued overnight for 13 h. As condensation droplets can be seen inside the flask, drying is continued for another 29 h.

The dry product is highly statically charged. The material is unloaded from the flask into a metal loading drum for solids in preparation for the next step of the reaction sequence.

A total of 10.5 kg of the product is obtained, representing a yield of 64% relative to C10 for dry content of the product.

### Step 2

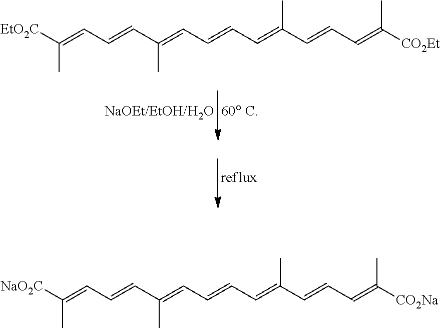

### Materials

| Compound | Mass or Volume | |
|---|---|---|
| bis-ethyl crocetinate (C20) made in Step 1 | 10.4 | kg |
| Sodium ethanolate 21% w/w in ethanol | 44 | L |
| Ethanol | 45 | L |
| Deionized water | 2.4 | L |
| Purified water | 250 | L |

10.38 kg of bis-ethyl crocetinate (C20) made in Step 1 are charged into a stainless steel reactor. 43 L of sodium ethanolate 21% w/w and 43 L of ethanol are aspirated into the right header and inerted. Direct addition from the right header into the reactor is barred. The mixture is transferred via 0.2/0.45 m inline filter into the left header and subsequently dropped into the reactor. The mixture is heated to Ti: 60° C. while the maximum jacket temperature is limited to 70° C. The mixture is stirred overnight at Tj: 60° C. for 14.5 h. The mixture is cooled to Ti: 40° C. for sampling.

The orange suspension is heated up to Ti: 60° C. and stirred overnight at Tj: 60° C. for 18 h before it is cooled to Ti: 40° C. for sampling.

The orange suspension is heated up to Ti: 60° C. and stirred overnight at Tj: 60° C. with increased stirring speed for 19 h before it is cooled to Ti: 40° C. for sampling.

250 L of purified water is degassed and added to the mixture. The reactor jacket limit is raised to 100° C. and the mixture heated to Ti: 80° C. It is then stirred overnight at Tj: 90° C. for 2.5 days using moderate stirring. The mixture is cooled to Ti: 40° C. for sampling.

The orange suspension is heated up to Ti: 80° C. and stirred at Tj: 90° C. for 1 h before cooling to Ti: 40° C. over 6 h, stirring at 40° C. for 30 minutes and heating up to Ti: 80° C. again over 3.5 h, stirring at Tj: 90° C. for 2 h before cooling down to Ti: 20° C. over 4.5 h and stirring at Ti: 20° C. for 1 h using slow stirring throughout.

The mixture is then discharged onto a pressure filter, fitted with a 12 m mesh filter cloth. Pressure is applied from the reactor only, as the glass filter cannot tolerate larger pressures. As the filtration is slow, suction is applied to the back end of the filter. This is initially done via a suction flask, but later replaced by suction from the header, as this allows suction to be maintained overnight. Filtration is complete after 3 days.

31 L inline filtered ethanol is charged into the reactor, followed by 10 L purified water. The mixture is piped onto the filter, again applying suction at the back end. Filtration is completed overnight.

30 L inline filtered ethanol is charged into the reactor, followed by 10 L purified water. The mixture is piped onto the filter, again applying suction at the back end. Filtration is completed overnight.

30 L inline filtered ethanol is charged into the reactor, followed by 10 L purified water. The mixture is piped onto the filter, again applying suction at the back end. Filtration is completed overnight.

32 L inline filtered ethanol is charged into the reactor, followed by 10 L purified water. The mixture is piped onto the filter, again applying suction at the back end. Filtration is completed after 3.5 h.

31 L inline filtered ethanol is charged into the reactor, followed by 11 L purified water. The mixture is piped onto the filter, again applying suction at the back end. Filtration is completed after 5.5 h.

31 L inline filtered ethanol is charged into the reactor, followed by 10 L purified water. The mixture is piped onto the filter, again applying suction at the back end. Filtration is completed overnight (15 h).

31 L inline filtered ethanol is charged into the reactor, followed by 10 L purified water. The mixture is piped onto the filter, again applying suction at the back end. Filtration is completed after 24 h.

32 L inline filtered ethanol is charged into the reactor, followed by 10 L purified water. The mixture is piped onto the filter, again applying suction at the back end. Filtration is completed after 2 h.

31 L inline filtered ethanol is charged into the reactor, followed by 10 L purified water. The mixture is piped onto the filter, again applying suction at the back end. Filtration is completed overnight (16 h).

31 L inline filtered ethanol is charged into the reactor, followed by 10 L purified water. The mixture is piped onto the filter, again applying suction at the back end. Filtration is completed overnight (21 h).

32 L inline filtered ethanol is charged into the reactor, followed by 10 L purified water. The mixture is piped onto the filter, again applying suction at the back end. Filtration is completed overnight (19 h).

30 L inline filtered ethanol is charged into the header, followed by 10 L purified water. The pH of the mixture is checked (pH: 6.50) and the solution charged into the reactor. The mixture is piped onto the filter, again applying suction at the back end. Filtration is completed overnight (23 h).

31 L inline filtered ethanol is charged into the header, followed by 10 L purified water. The solution is charged into the reactor and piped onto the filter, again applying suction at the back end. Filtration is completed overnight (21 h).

43 L inline filtered ethanol is charged into the reactor and piped onto the filter, again applying suction at the back end. Filtration is completed overnight (16 h).

The filter cake is dried at reduced pressure under nitrogen flow for 45 h.

As the material is transferable, it is taken out of the filter and placed into a 100 L flask for drying on a rotary evaporator at Tj: 55° C. at reduced pressure (p: 262-13 mbar). Drying is continued for 10 days, 22 hours.

A 1 mm sieve is fitted a sieve mill. 8.3 kg of orange solid with lumps is transferred to the sieve mill and the material milled within 31 minutes using 724-810 rpm paddle speed and 0.1 cm offset over the sieve. 8.2 kg of milled material is obtained and drying on the rotary evaporator is continued for 5 days, 8 hours. The vacuum is lowered to 13 mbar by deploying a more powerful pump and the bath temperature increased to Tj: 60° C. Drying is continued for 41 hours.

8.154 kg of trans sodium crocetinate is obtained, representing a yield of 84% relative to bis-ethyl crocetinate (C20). HPLC: 98.3% w/w. Karl-Fischer: 2.5% w/w. Na content by FAAS: 12.3%.

### Example 4

Trans sodium crocetinate is mixed with various solvents under various conditions as shown in Table 1 (refer to Example 1 for representative procedures).

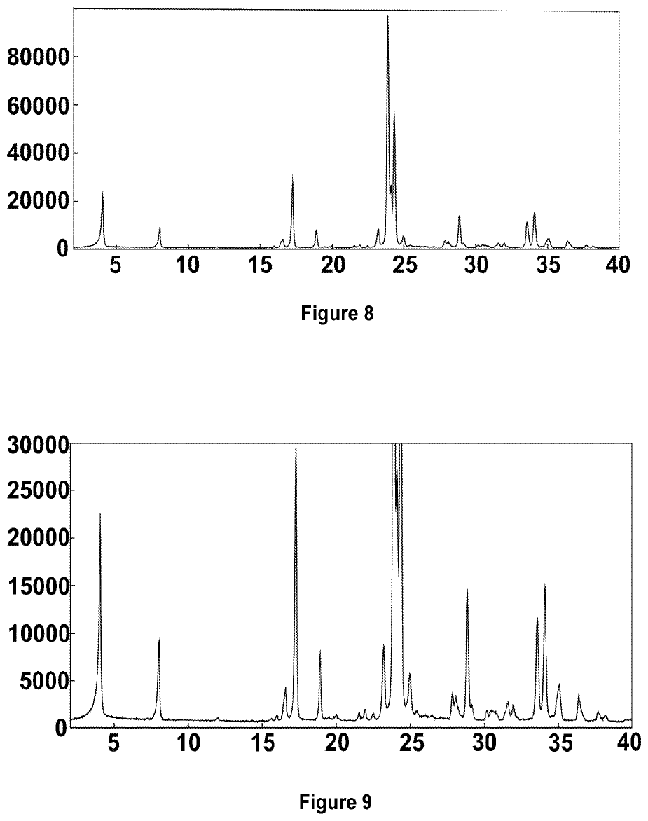

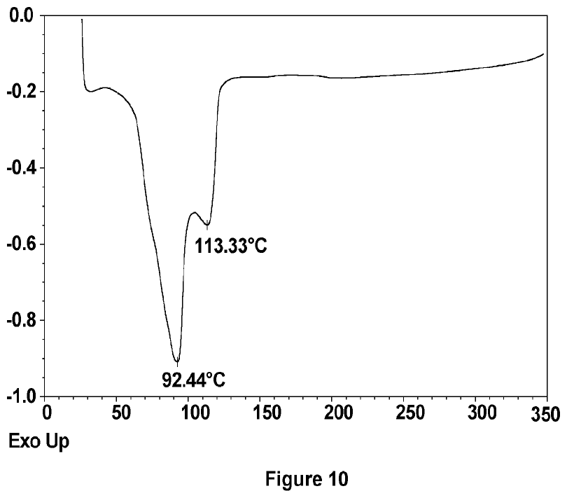

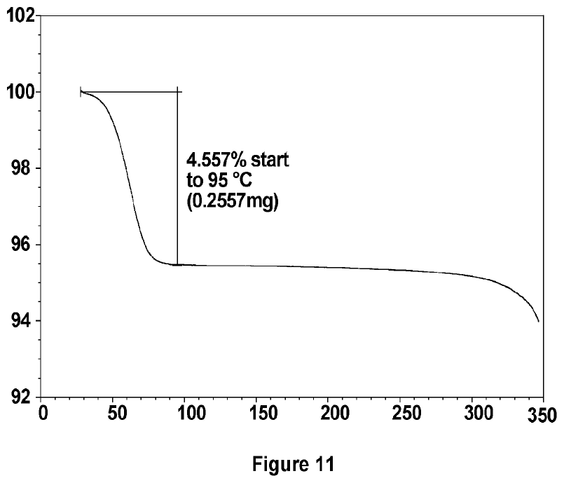

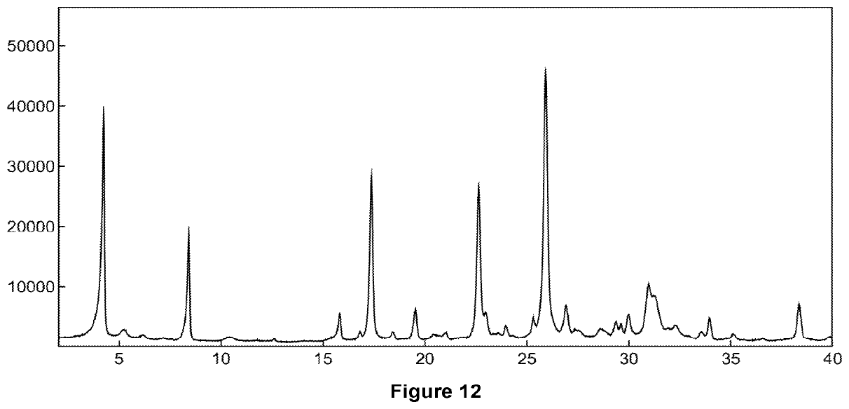

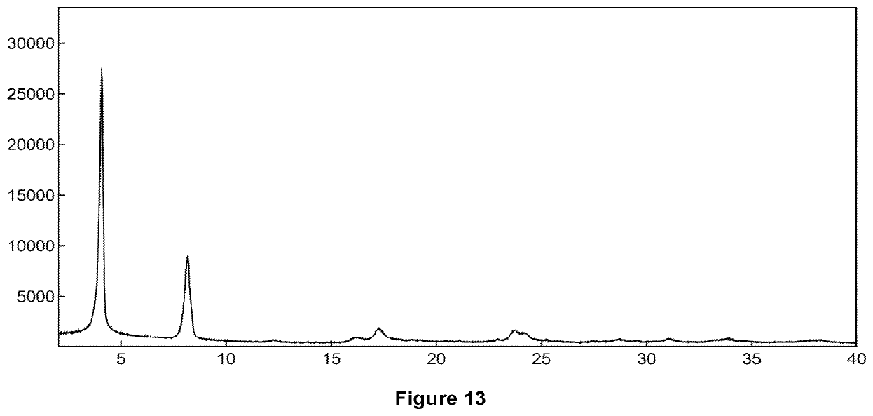

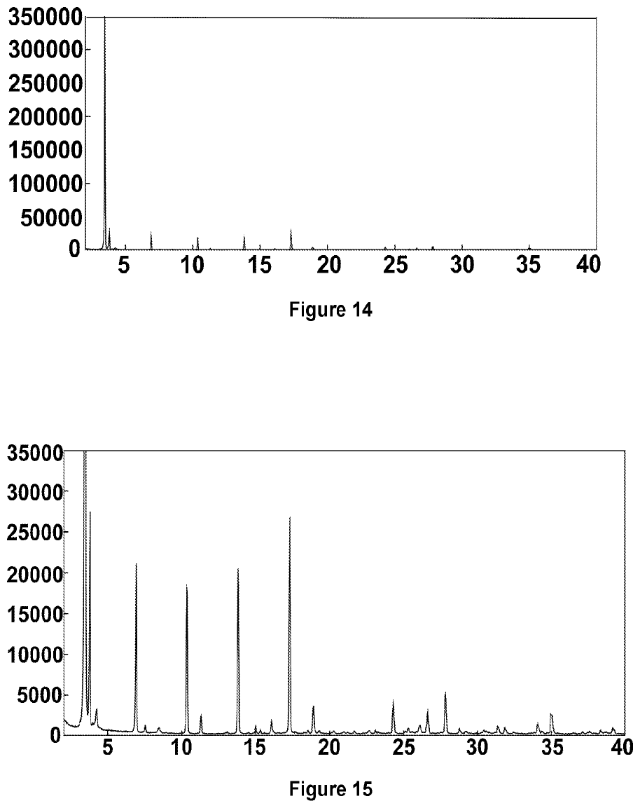

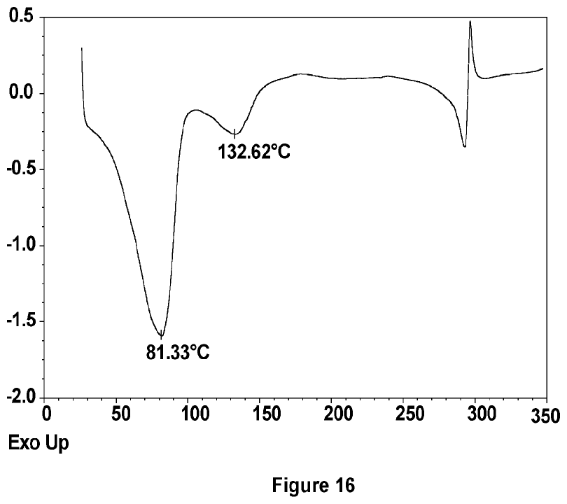

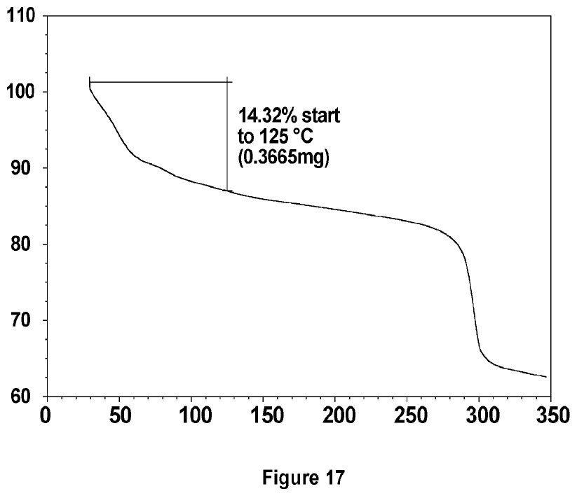

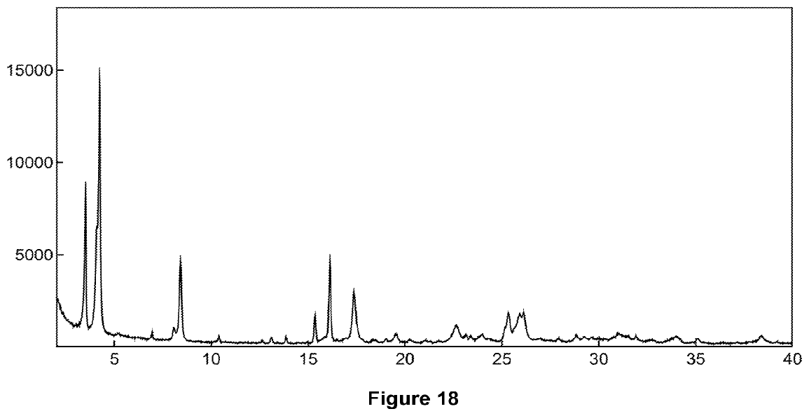

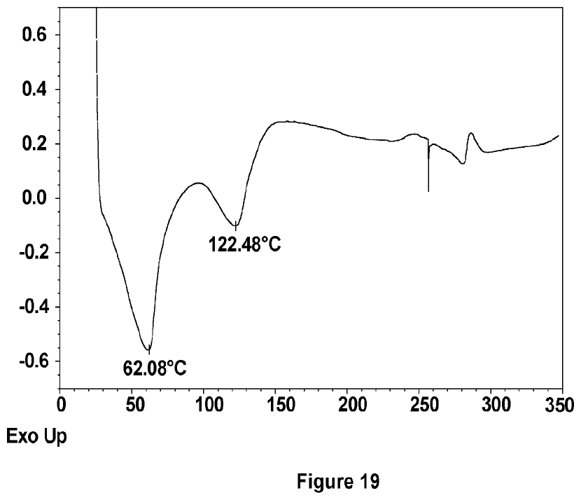

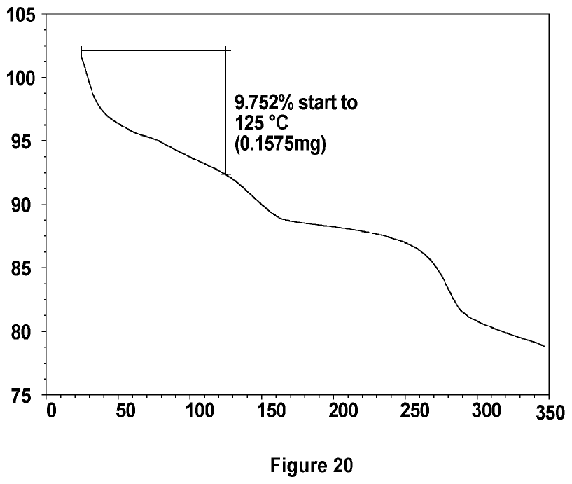

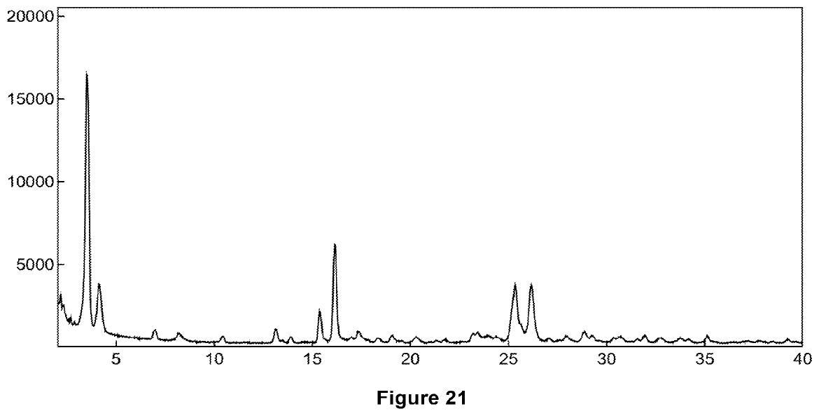

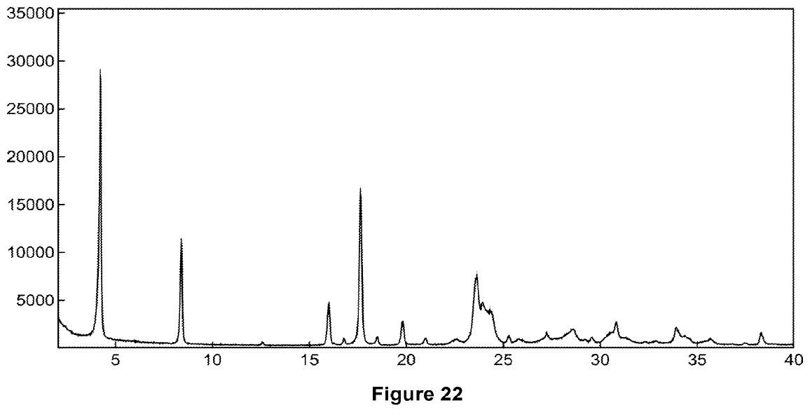

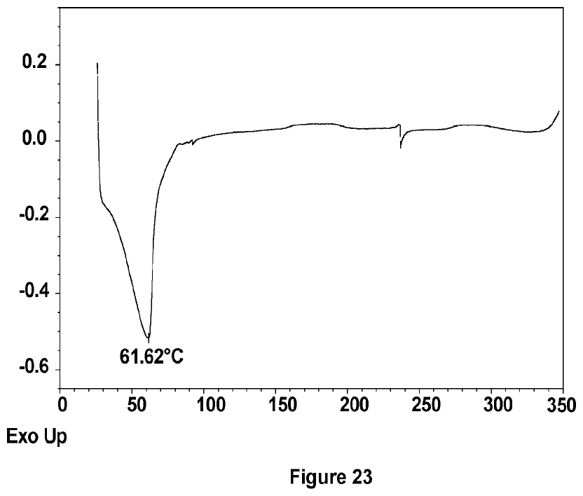

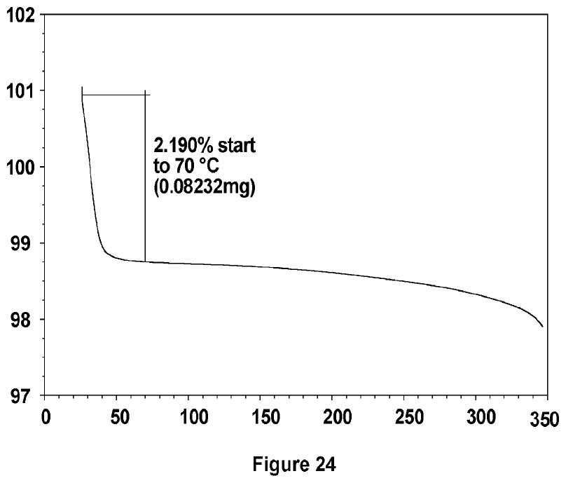

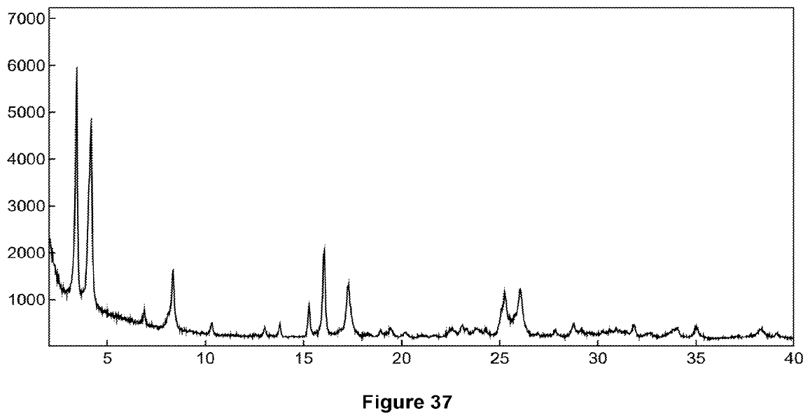

TABLE 1

| Method | Solvent | Conditions^{a} | Crystal Form^{b} |
|---|---|---|---|
| Cooling | acetone/water (20:80) | 60° C. to 2° C. | D (Example 7, XRPD |
| | | | is shown in FIG. 18, |
| | | | DSC is in FIG. 19, |
| | | | and TGA is in FIG. 20) |
| | | | D |
| | | | D |
| | acetone/water (50:50) | 60° C. to −15° C. | B |
| | | | B |
| | | | B |
| | | | B |
| | | | B (XRPD is shown in |
| | | | FIG. 32 and FIG. 33) |
| | | | B (XRPD is shown in |
| | | | FIG. 34 and FIG. 35) |
| | IPA/water (50:50) | 60° C. to 2° C. | B (starting material |
| | | | for Example 9 and |
| | | | Example 12, XRPD is |
| | | | shown in FIG. 7) |
| | MeOH/water (40:60) | 60° C. to 2° C. | B |
| | THF/water (50:50) | 60° C. to 2° C. | C (Example 1G and |
| | | | Example 6, XRPD is |
| | | | shown in FIG. 14, |
| | | | DSC is in FIG. 16, |
| | | | and TGA is in FIG. 17) |
| | | | C |
| | water | 60° C. to 2° C. | D (XRPD is shown in |
| | | | FIG. 37) |
| | | | D |
| | | | D |
| Slurry | acetone | 40° C., 6 days | A (br) |
| | ACN | 40° C., 6 days | A |
| | 1,4-dioxane | 40° C., 6 days | A (br) |
| | DMF | 40° C., 6 days | A (br) |
| | EtOH | 40° C., 6 days | A |
| | EtOAc | 40° C., 6 days | A |
| | Et₂O | 40° C., 6 days | A |
| | IPA | 40° C., 6 days | A |
| | MeOH | 40° C., 6 days | A |
| | MEK | 40° C., 6 days | A |
| | THF | 40° C., 6 days | A |
| | water | RT, 10 days | A |
| | acetone/water (20:80) | RT, 10 days | A |
| | acetone/water (20:80) | RT, 5 days | A |
| | acetone/water (25:75) | RT, 5 days | A |
| | acetone/water (30:70) | RT, 5 days | A + NC |
| | acetone/water (35:65) | RT, 5 days | A |
| | acetone/water (40:60) | RT, 5 days | A |
| | acetone/water (45:55) | RT, 5 days | A |
| | acetone/water (50:50) | RT, 10 days | B (Example 1H and |
| | | | Example 5, XRPD is |
| | | | shown in FIG. 8, |
| | | | DSC is in FIG. 10, |
| | | | and TGA is in FIG. 11) |
| | acetone/water (50:50) | RT, 5 days | A |
| | acetone/water (95:5) | 40° C., 6 days | A |
| | ACN/water (50:50) | RT, 10 days | A |
| | ACN/water (95:5) | 40° C., 6 days | A |
| | dioxane/water (60:40) | RT, 10 days | B + A (tr) |
| | dioxane/water (95:5) | 40° C., 6 days | A |
| | EtOH/water (95:5) | 40° C., 6 days | A |
| | IPA/water (50:50) | RT, 10 days | A |
| | IPA/water (95:5) | 40° C., 6 days | A |
| | MeOH/CHCl₃ (1:1) | 40° C., 6 days | A |
| | MeOH/water (40:60) | RT, 10 days | A |
| | MeOH/water (95:5) | 40° C., 6 days | A |
| | THF/water (95:5) | 40° C., 6 days | A |
| Grind | acetone/water | 100% powder, 30 min | A (Example 1I, XRPD |
| | | | is shown in FIG. 12) |
| | ACN/water | 100% powder, 30 min | A |
| | DCM | 100% powder, 30 min | A |
| | dioxane | 100% powder, 30 min | A |
| | EtOAc | 100% powder, 30 min | A |
| | EtOH/water | 100% powder, 30 min | A |
| | MeOH/water | 100% powder, 30 min | A |
| | IPA/water | 100% powder, 30 min | A |
| | MEK | 100% powder, 30 min | A |
| | THF | 100% powder, 30 min | A |
| Lyophilization | water | Stirred in acetone, | A (LC) |
| followed by | | RT, 5 days | |
| slurry of | | Stirred in ACN, RT, 5 | A (LC) |
| lyophile | | days | |
| | | Stirred in dioxane, | A (LC) |
| | | RT, 5 days | |
| | | Stirred in EtOAc, RT, | A (LC) |
| | | 5 days | |
| | | Stirred in EtOH, RT, | A (LC) |
| | | 5 days | |
| | | Stirred in IPA, RT, 5 | A (LC) |
| | | days | |
| | | Stirred in MEK, RT, 5 | A (LC) |
| | | days | |
| | | Stirred in MeOH, RT, | E (Example 1J and |
| | | 5 days | Example 8, XRPD is |
| | | | shown in FIG. 22 |
| | | | DSC is in FIG. 23, |
| | | | and TGA is in FIG. 24) |
| | | | E |
| | | | E |
| | | Stirred in MTBE, RT, | A (LC) |
| | | 5 days | |
| | | Stirred in THF, RT, 5 | A (LC) |
| | | days | |
| | | No post-lyophilization | A (LC) |
| | | slurry | A (LC) |
| | | | A (LC) |
| Antisolvent | acetone/water (20:80) | RT, acetone to 2° C. | B |
| Precipitation | | RT, acetone | B |
| | water | RT, ACN AS to 2° C. | C + D (tr) |
| | | RT, dioxane AS | B (LC) (Example 1K, |
| | | | XRPD is shown in |
| | | | FIG. 13) |
| | | RT, EtOH AS | B + peaks |
| | | RT, IPA AS | B (XRPD is shown in |
| | | | FIG. 30 and FIG. 31) |
| Evaporation | acetone/water (20:80) | RT | D (Example 1L, XRPD |
| | | | is shown in FIG. 21) |
| | acetone/water (50:50) | RT | D |
| | ACN/water (50:50) | RT | D |
| | dioxane/water (60:40) | RT | LC |
| | IPA/water (50:50) | RT | B + C |
| | MeOH/water (40:60) | RT | D |
| | THF/water (50:50) | RT | D |
| | water | RT | D |

^{a}AS— anti-solvent; RH = relative humidity, RT = room temperature
^{b}Br = broad XRPD peaks; LC = low crystallinity; NC = non-crystalline; pks = peaks from an unknown source; tr = trace

### Example 5—Trans Sodium Crocetinate (Form B)

Sample preparation is carried out in a nitrogen glove box. 26.1 mg of TSC from Example 2 is placed in a 1-dram glass vial with 0.45 mL of 50:50 acetone:water (v/v). The slurry is stirred magnetically at room temperature for 10 days. The sample is centrifuged and the mother liquor is decanted. The resulting solid is allowed to air dry in a nitrogen glove box at room temperature prior to XRPD analysis. An XRPD of the product is shown in FIG. 8 (refer to Example 1 for XRPD details). FIGS. 10 and 11 depict differential scanning calorimetry (DSC) analysis and thermogravimetric analysis (TGA), respectively, for the product (refer to Example 1 for experimental details).

### Example 6—Trans Sodium Crocetinate (Form C)

Sample preparation is carried out in a nitrogen glove box. 25.6 mg of TSC from Example 2 is placed in a 1-dram glass vial with 1.0 mL of 50:50 THF:water (v/v). The slurry is stirred magnetically on a 60° C. hot plate. Solid remains after approximately 2 hours, so an additional 1.5 mL of solvent is added. A clear orange solution results. The heat is turned off and the sample is allowed to cool to room temperature. No solid is observed after 1 day at room temperature, so the sample is moved to a refrigerator (˜2-5° C.). Solid is observed after 17 days in the refrigerator. The sample is centrifuged, the mother liquor is decanted, and the solid is allowed to air dry in a nitrogen glove box at room temperature prior to XRPD analysis. An XRPD of the product is shown in FIG. 14 (refer to Example 1 for XRPD details). FIGS. 16 and 17 depict differential scanning calorimetry (DSC) analysis and thermogravimetric analysis (TGA), respectively, for the product (refer to Example 1 for experimental details).

### Example 7—Trans Sodium Crocetinate (Form D)

Sample preparation is carried out in a nitrogen glove box. 26.0 mg of TSC from Example 2 is placed in a 1-dram glass vial with 1.0 mL of 20:80 acetone:water (v/v). The slurry is stirred magnetically on a 60° C. hot plate. Solid remains after approximately 2 hours, so an additional 2.5 mL of solvent is added. A clear orange solution results. The heat is turned off and the sample is allowed to cool to room temperature. A very small amount of solid is observed after 1 day at room temperature, so the sample is moved to a refrigerator (˜2-5° C.). After 1 day, the amount of solid has not increased, so the sample is placed in a freezer (˜−20° C.). Additional solid forms after 17 days. The sample is centrifuged, the mother liquor is decanted, and the solid is allowed to air dry in a nitrogen glove box at room temperature prior to XRPD analysis. An XRPD of the product is shown in FIG. 18 (refer to Example 1 for XRPD details). FIGS. 19 and 20 depict differential scanning calorimetry (DSC) analysis and thermogravimetric analysis (TGA), respectively, for the product (refer to Example 1 for experimental details).

### Example 8—Trans Sodium Crocetinate (Form E)

Sample preparation is carried out in a nitrogen glove box. 26.8 mg of TSC from Example 2 is placed in a glass vial with 8 mL of water. The vial is sonicated to dissolve the solid. The solution is transferred to a 50 mL round bottom flask which is subsequently rotated in a dry-ice bath to freeze the solution to the sides of the flask wall. The flask is then placed on a LabConco FreeZone 1 lyophilizer. Solid is present the next day. The solid is transferred to a 2-dram glass vial and 6 mL of methanol is added. The slurry is stirred magnetically at room temperature for 4 days. The sample is centrifuged and the mother liquor is decanted. The resulting solid is allowed to air dry in a nitrogen glove box at room temperature prior to XRPD analysis. An XRPD of the product is shown in FIG. 22 (refer to Example 1 for XRPD details). FIGS. 23 and 24 depict differential scanning calorimetry (DSC) analysis and thermogravimetric analysis (TGA), respectively, for the product (refer to Example 1 for experimental details).

### Example 9—Trans Sodium Crocetinate (Form F)

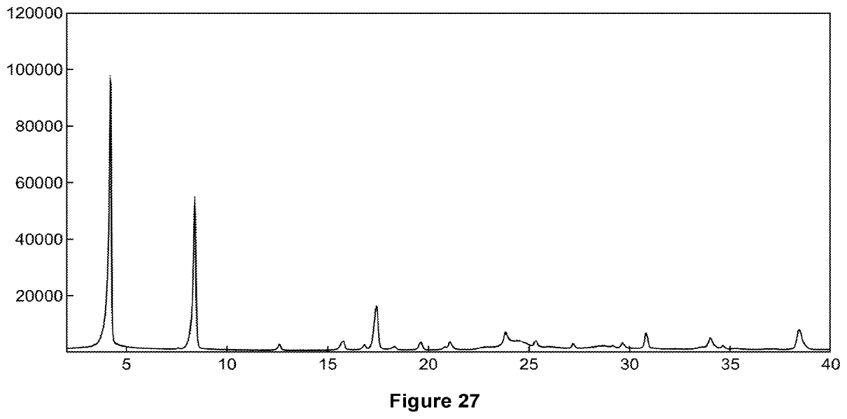

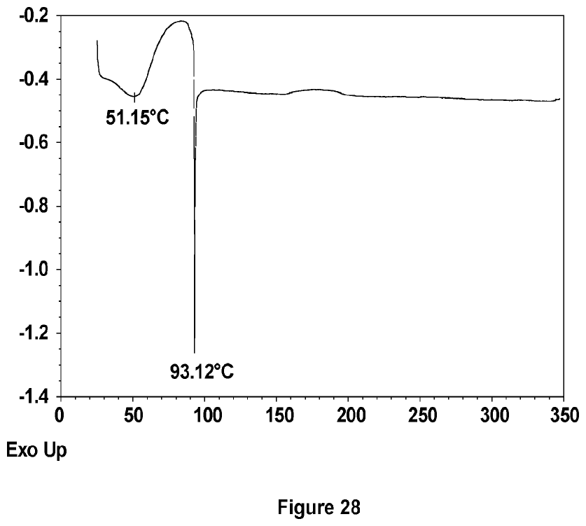

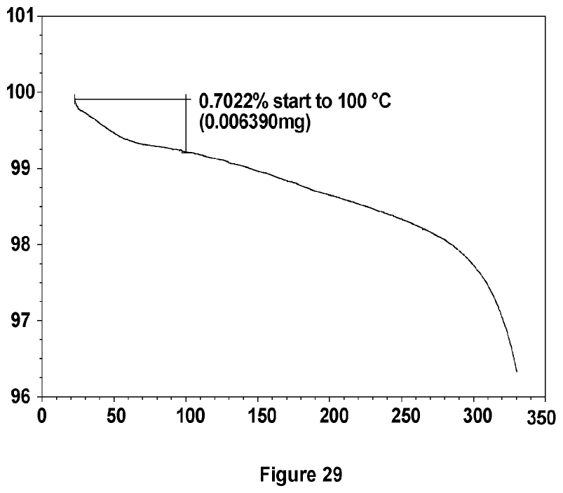

Into a platinum TGA pan is placed 6.8 mg of TSC Form B. The pan is placed on a TGA and heated to 100° C. and held for 1 minute. The resulting solid is analyzed by XRPD. An XRPD of the product is shown in FIG. 27 (refer to Example 1 for XRPD details).

### Example 10—Competitive Slurry Experiments

Competitive slurry experiments involving Crystalline Forms A through E are carried out at three different temperatures using three different solvent systems (Table 2). In the case of aqueous solvent systems, Crystalline Form B appears to be the most stable form. In the non-aqueous system, Crystalline Form A appears to be the most stable. At 50° C., the slurries in 1:1 acetone:water and 1:1 acetonitrile:water produce a mixture of Crystalline Forms A and B and Crystalline Form A, respectively. Although both solvent systems contain significant amounts of water, the slurries are carried out at a temperature at which Crystalline Form B is known to lose volatiles. The TG analysis of Crystalline Form B shows that volatile loss occurs between approximately 50 and 75° C. That is probably the reason that Crystalline Form A results from the higher-temperature slurries in water-containing solvents.

TABLE 2
Competitive Slurries

| Solvent | Temperature (° C.) | XRPD Pattern |
|---|---|---|
| MeOH | 2 | A |
| acetone:water (1:1) | | B |
| ACN:water (1:1) | | B |
| MeOH | 25 | A |
| acetone:water (1:1) | | B |
| ACN:water (1:1) | | B |
| MeOH | 50 | A |
| acetone:water (1:1) | | A + B |
| ACN:water (1:1) | | A |

### Example 11—Competitive Slurry Experiments

Additional competitive slurry experiments are carried out between Crystalline Forms A, B, and F (Table 3). The data are consistent with the results in Example 10. Crystalline Form A is the most stable form under nonaqueous conditions and Crystalline Form B is the most stable form under aqueous conditions. The elevated temperature aqueous slurry data is also consistent with Example 10. Crystalline Form A is observed in aqueous media at 50° C. In the case of the 1:1 ethanol:water slurry, the XRPD pattern of the resulting material matches that of Crystalline Form A but contains several unidentified peaks.

TABLE 3

| Solvent | Temperature (° C.) | XRPD Pattern |
|---|---|---|
| EtOH (absolute) | 2 | A |
| EtOH:MeOH (90:10) | | A |
| EtOH:toluene (90:10) | | A |
| EtOH:water (1:1) | | B |
| EtOH (absolute) | 25 | A |
| EtOH:MeOH (90:10) | | A |
| EtOH:toluene (90:10) | | A |
| EtOH:water (1:1) | | B |
| EtOH (absolute) | 55 | A |
| EtOH:MeOH (90:10) | | A |
| EtOH:toluene (90:10) | | A |
| EtOH:water (1:1) | | A + peaks |

### Example 12—Heating

Crystalline Forms A and B are heated to 100° C. in a TG furnace, held at that temperature for 1 minute, and immediately analyzed by XRPD. The data are summarized in Table 4. Crystalline Form A remains unchanged after heating, but Crystalline Form B converts to Crystalline Form F.

TABLE 4

| Starting Material | Conditions | XRPD pattern |
|---|---|---|
| Crystalline Form A from | Heat to 100° C., hold 1 | A |
| Example 2 | minute | |
| Crystalline Form B | | F |

### Example 13—Stability Studies

### Physical Stability

### Solid State

The solid-state stability of TSC Crystalline Forms A and F are evaluated at 25° C./60% relative humidity (RH) and 40° C./75% RH. The data are summarized in Table 5. The data show that both forms remain unchanged for up to 4 weeks under the testing conditions.

To a 1-dram glass vial is added approximately 20 mg of TSC Crystalline Form A or F. The open vials are placed in relative humidity (RH) jars. The RH in each jar is controlled by a saturated salt solution (sodium bromide for 60% RH and sodium chloride for 75% RH). The RH jars are purged with nitrogen and sealed. The sealed RH jars are placed in ovens set at the appropriate temperatures. The materials are analyzed at given time points by XRPD.

TABLE 5

| Starting Material | Conditions | Time (weeks) | XRPD Result |
|---|---|---|---|
| Crystalline | 25° C./60% RH | 1 | A |
| Form A from | | 2 | A |
| Example 2 | | 4 | A |
| | 40° C./75% RH | 1 | A |
| | | 2 | A |
| | | 4 | A |
| Crystalline | 25° C./60% RH | 1 | F |
| Form F | | 2 | F |
| | | 4 | F |
| | 40° C./75% RH | 1 | F |
| | | 2 | F |
| | | 4 | F |

### Slurry Conditions

The physical stability of Crystalline Form F is evaluated when it is slurried in three solvent systems (anhydrous ethanol and ethanol containing both 1 and 5% water by volume) at ambient temperature. The data are summarized in Table 6. The data show that Crystalline Form F remains unchanged for up to 4 days under the testing conditions.

To a 1-dram glass vial is added approximately 20 mg of TSC Crystalline Form F and 1 mL of a given solvent. The slurry is stirred at room temperature for a given amount of time. The vial is then centrifuged, the mother liquor decanted, and the wet solid is analyzed by XRPD.

TABLE 6

| Starting Material | Solvent | Time (days) | XRPD Result |
|---|---|---|---|
| Crystalline | Ethanol | 1 | F |
| Form F | (anhydrous) | 2 | F |
| | | 4 | F |
| | Ethanol | 1 | F |
| | (1% water v/v) | 2 | F |
| | | 4 | F |
| | Ethanol | 1 | F |
| | (5% water v/v) | 2 | F |
| | | 4 | F |

### Example 14—Trans Sodium Crocetinate (Form B)

Sample preparation is carried out in a nitrogen glove box. To a 5-dram glass vial is added 25.2 mg of TSC from Example 2 and 4.0 mL of 1:1 acetone:water (v/v). The sample is stirred on a 60° C. hot plate and the solid dissolves. The heat is turned off and the sample is allowed to cool to room temperature. A small amount of solid is observed in the sample after 1 day, and it is placed in a refrigerator (˜5° C.). After 1 day, the amount of solid has not increased, so the sample is placed in a freezer (˜−20° C.). Additional solid forms after 8 days, so the sample is centrifuged, the mother liquor decanted, and the solid allowed to dry in a nitrogen glove box. The resulting solid is identified as Form B by XRPD analysis.

### Example 15—Trans Sodium Crocetinate (Form C)

Sample preparation is carried out in a nitrogen glove box. To a 1-dram glass vial is added 25.0 mg of TSC from Example 2 and 2.0 mL of 1:1 tetrahydrofuran:water (v/v). The sample is stirred on a 60° C. hot plate and the solid dissolves. The heat is turned off and the sample is allowed to cool to room temperature. A small amount of solid is observed in the sample after 1 day, and it is placed in a refrigerator (˜5° C.). After 1 day, the amount of solid has not increased, so the sample is placed in a freezer (˜−20° C.). Additional solid forms after 8 days, so the sample is centrifuged, the mother liquor decanted, and the solid allowed to dry in a nitrogen glove box. The resulting solid is identified as Form C by XRPD analysis.

### Example 16—Trans Sodium Crocetinate (Form D)

Sample preparation is carried out in a nitrogen glove box. To a 5-dram glass vial is added 26.7 mg of TSC from Example 2 and 3.5 mL of 1:4 acetone:water (v/v). The sample is sonicated and the solid dissolves. The vial is left uncapped and the solvent is allowed to evaporate at room temperature in a nitrogen glove box. The resulting solid is identified as Form D by XRPD analysis.

### Example 17—Trans Sodium Crocetinate (Form E)

Sample preparation is carried out in a nitrogen glove box. A solution of 25.2 mg of TSC from Example 2 in 8.0 mL of water is placed in a 50-mL round bottom flask. The flask is rotated in a dry ice acetone flask to freeze the solution to the sides. The flask is placed on a Labconco FreeZone 1 lyophilizer overnight. The dried lyophile is transferred to a 5-dram vial. To the vial is added 6 mL of methanol and the resulting slurry is stirred at room temperature for 5 days. The sample is centrifuged, the mother liquor decanted, and the solid allowed to dry in a nitrogen glove box. The resulting solid is identified as Form E by XRPD analysis.

### Example 18—Trans Sodium Crocetinate (Form F)

39.8 g of trans sodium crocetinate is dissolved in 8 V n-BuOH, 2 V EtOH, 38.7 V water, and 0.3 V NaOH 30%. Initially, this gives a biphasic mixture. 200 mL 25% EtOH is added portion wise until the mixture is a single phase. After filtration, the solution is concentrated to 11 L/kg (using 3×100 mL EtOH (total of 7.5V) to control foaming) and then diluted with 30 L/kg EtOH. The product suspension is filtered, washed with 4 L/kg 50% EtOH, and dry washed with 10 L/kg EtOH to give 32.5 g of solid.

### Example 19—Trans Sodium Crocetinate (Form F)

40 g of trans sodium crocetinate is dissolved in 8 V n-BuOH, 2 V EtOH, 38.7 V water, and 0.3 V NaOH 30%. Initially, this gives a biphasic mixture. After filtration, the solution is concentrated to 11 L/kg at 50° C. under vacuum (250 mL (8.25 V). EtOH is added in portions to counter foaming, giving a suspension, which is diluted (50° C.) with 30 L/kg EtOH. The product suspension is filtered, washed with 4 L/kg 50% EtOH, and dry washed with 10 L/kg EtOH to give 34.6 g of solid.

### Example 20—Trans Sodium Crocetinate (Form F+Trace A)

50 g of C20 (see Examples 2 and 3 for structure), 500 mL 1-BuOH (10 V), 500 mL EtOH (10 V), and 50 mL NaOH 30% w/w (1 V, 4 equivalents) are stirred in a 3 L round bottom flask at Ta: 50° C. and conversion of C20 is monitored. After C20 conversion, the mixture is concentrated on a rotary evaporator to 10 V (480 g) to give a thick suspension. 1200 mL water (24 V) is added leading to almost complete dissolution and a small organic phase. Mixture is stirred overnight at Ta: 50° C. Mixture is filtered. The filtrate is concentrated to 11 V and diluted slowly with 350 mL EtOH over 1 hour to give a suspension (1727 g). Mixture is stirred slowly overnight, then concentrated at 50° C., 130 mbar, resulting in 549 g suspension. Mixture is diluted with 1500 mL EtOH and allowed to cool over 2.5 hours. Mixture is filtered and then washed with 2×500 mL EtOH. After drying, 42.6 g is obtained. Karl-Fischer: 0.4% w/w.

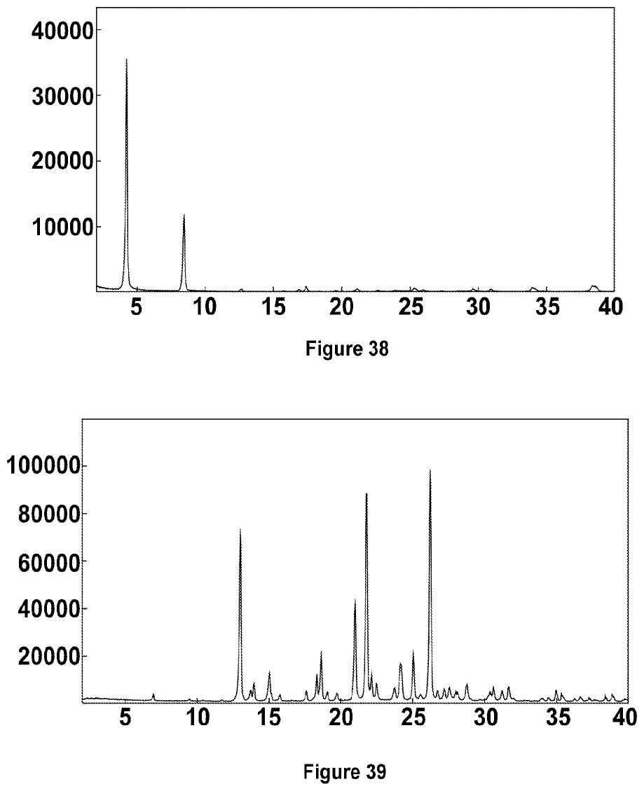

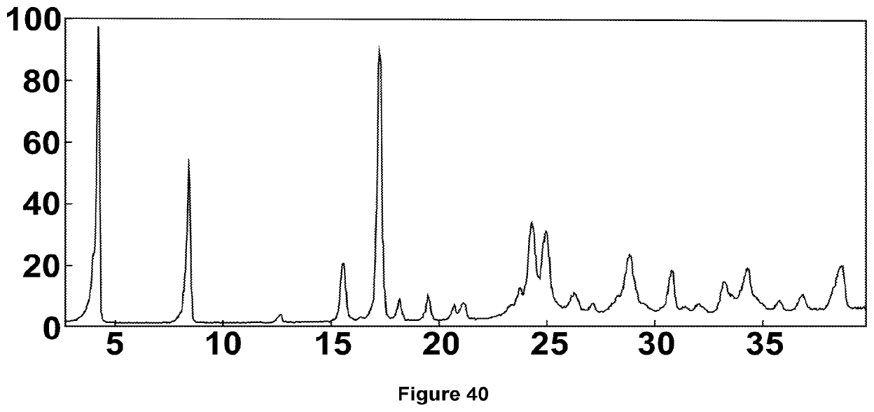

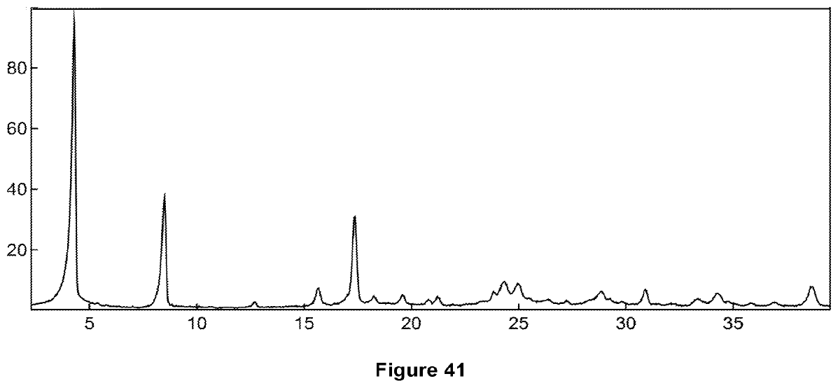

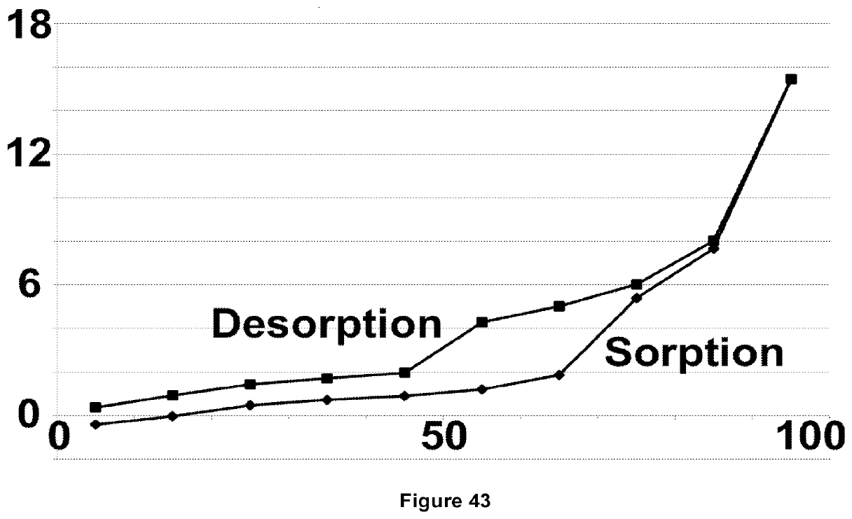

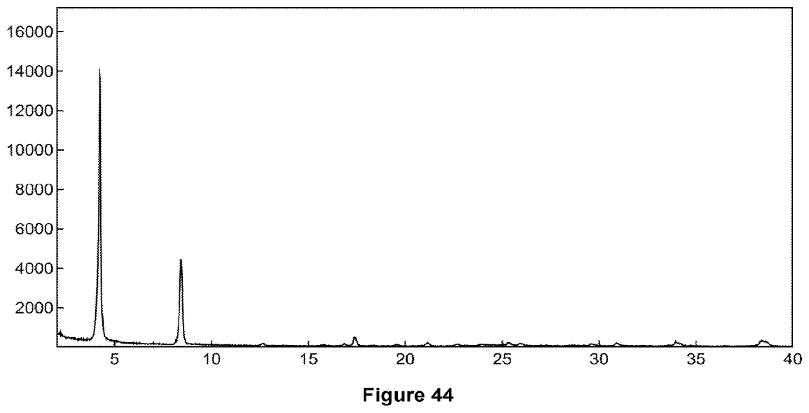

40.7 of the solid is dissolved in 325 mL n-BuOH (8 V), 81.4 mL EtOH (2 V), 1575 mL water (38.7 V), and 12.2 mL (16.2 g) NaOH 30% (0.3 V). After filtration, the solution is concentrated to 11 L/kg at 50° C. under vacuum (2×250 mL EtOH is added in portions over 20 minutes to counter foaming), and diluted at 50° C. with 30 L/kg EtOH. The product suspension is filtered, washed with 4 L/kg 50% EtOH, and dry washed with 10 L/kg EtOH to give 35.5 g of solid. An XRPD of the product is shown in FIG. 38 (refer to Example 1 for XPRD details). FIG. 43 depicts a dynamic vapor sorption isotherm of the product (refer to Example 1 for experimental details). An XRPD of the material after a dynamic vapor sorption analysis (refer to Example 1 for details) is in FIG. 44. The material after the DVS experiment remains unchanged by XRPD.

### Example 21—Trans Sodium Crocetinate (Form F+B)

200 g of trans sodium crocetinate, 8 V BuOH, 2 V EtOH, 38.7 V water, and 0.3 V NaOH 30% at 50° C. are concentrated over 2 days in a 10 L double jacketed reactor. 5 V EtOH is added in portions of 250 mL as entrainer to reach a final volume of 11 V. Mixture is warmed to 50° C. and 30 V EtOH is added over 90 minutes. Mixture is cooled to 20° C. over 4 h and filtered. Reactor is rinsed using 4 V 50% EtOH. Product is dried for 2 days on filter under N₂. 178.2 g obtained; dry content: 99.0% w/w.

200 g of trans sodium crocetinate, 8 V BuOH, 2 V EtOH, 38.7 V water, and 0.3 V NaOH 30% are at 50° C. and form a suspension. 600 mL additional EtOH gives a clear solution. Mixture is concentrated over 2 days in a 10 L double jacketed reactor with the jacket at 50° C., 50-70 mbar. 11 V EtOH is added in portions of 250-500 mL as entrainer to reach a final volume of 11 V. Mixture is warmed to 50° C. and 30 V EtOH is added over 90 minutes. Mixture is cooled to 20° C. overnight (18 h) and filtered. Reactor is rinsed using 4 V 50% EtOH. Product is dried for 3 days (w/e) on filter under N₂. 178.5 g obtained.

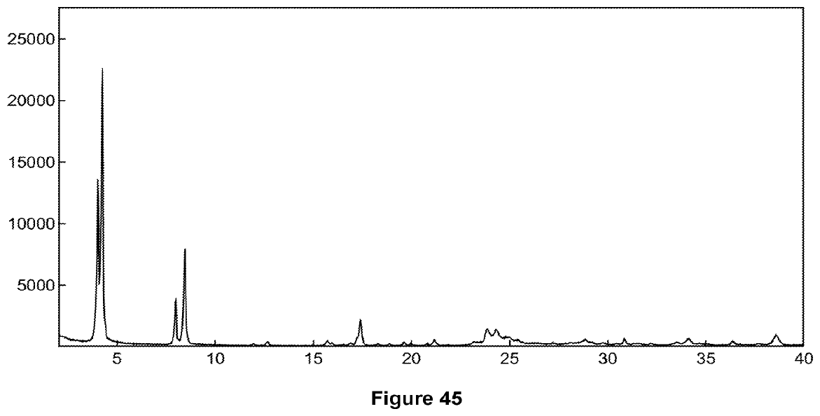

Product is used with product from 178.2 g batch above in slurry wash with 3.2 L (8 V with 2×about 200 g of trans sodium crocetinate) 50% EtOH at 20° C. Slurry once more and finally slurry in 4.0 L EtOH at 20° C., filter, wash with 1.6 V EtOH, and dry on filter under N₂ for 4 days to give 320.3 g; 80% yield. HPLC assay (as is): 96.5% w/w. Karl-Fischer: 1.5% w/w. An XRPD of the product is shown in FIG. 45 (refer to Example 1 for XPRD details).

### Example 22—Trans Sodium Crocetinate (Form J)

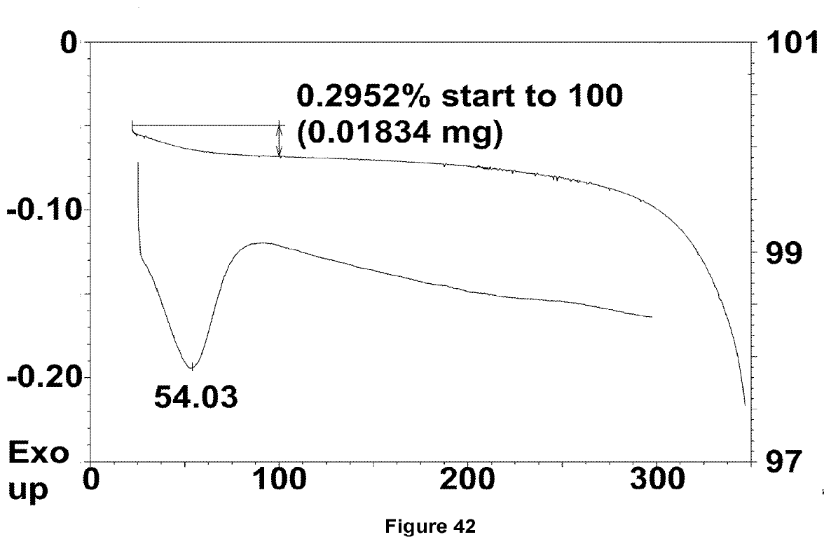

Into a platinum TGA pan is placed 6.8 mg of TSC Form F+Form B from Example 21. The pan is placed on a TGA and heated to 100° C. and held for 5 minutes. The resulting solid is analyzed by XRPD. An XRPD of the product is shown in FIG. 39 (refer to Example 1 for XRPD details). FIG. 42 depicts a differential scanning calorimetry (DSC) and thermogravimetric analysis (TGA) overlay for the product.

### Example 23—Equilibrium Solubility

Solubility is evaluated using shake-flask method at room temperature. Individual samples are prepared as follows: Approximately 20 mg of TSC Forms A, F, and J are placed in a 1-dram glass vial. To each vial is added 1.5 mL of media. The vials are then placed in an incubation chamber at room temperature with shaking at 150 rpm. After 24 hours the vials are removed and each slurry is vacuum filtered. The mother liquor is diluted 1:10 with the respective media for that sample and analyzed by HPLC. Water used is HPLC grade. Ethanol used in absolute, HPLC grade.

TABLE 7

| Starting Material | Media | XRPD Result |
|---|---|---|
| Crystalline Form A | Water | A |
| | EtOH | A |
| Crystalline Form F | Water | F |
| (from Example 20) | EtOH | F |
| Crystalline Form J | Water | Similar to A |
| | EtOH | Similar to F |

After 24-hour slurry experiments Crystalline Forms A and F remain unchanged in water and ethanol, while Crystalline Form J exhibits form conversion. In experiments with water, HPLC chromatograms show impurities.

### Example 24—Trans Sodium Crocetinate (Forms F+B)

### Step 1

Amounts are L/kg related to 2,7-dimethyl-2,4,6-octatrien-1,8-dial (C10) (3 kg). Charge reactor with 6 equivalents of standard grade NaOAc (9 kg), 2,7-dimethyl-2,4,6-octatrien-1,8-dial (C10), and C5 Wittigester bromide (C5) (20.4 kg, 2.38 equivalents). See Examples 2 and 3 for structures of C5 and C10. Slurry in 8 L/kg acetonitrile (24 L). Add deionized water (0.033 kg, 0.1 equivalent). Heat mixture to Ti: 80° C. Stir mixture at Ta: 85° C. for 15-72 hours.

For work-up, add 30 L deionized water (10 L/kg). Stir mixture at mixture at Ti: 80° C. for at least 24 hours. Cool to 60° C. over at least 20 minutes. Separate phases (organic phase remains, aqueous phase is removed). Add ethanol (19 kg, 8 L/kg) and deionized water (12 L, 4 L/kg). Stir at Ti: 80° C. for at least 48 hours. Cool to Ti: 60° C. over at least 20 minutes and then filter suspension. Charge deionized water (6 L) and EtOH (6 L). Stir for at least 5 minutes at Ta: 60° C. Filter suspension and dry filter cake until dry content ≥50% w/w and transferable. Transfer the wet cake to the reactor. Slurry in ethanol:deionized water 1:1 (15 L each) and stir for at least 10 minutes at Ti: 20° C. Filter suspension. Rinse the reactor with deionized water and EtOH (6 L each) and stir for at least 5 minutes at Ta: 20° C. Filter suspension. Dry filter cake at Ta: 40-50° C., p≤0.2 bar for >5 h and until dry content ≥90% w/w. Transfer cake into rotary flask and dry with rotovap at Ta: 60° C., p: 200-0 mbar for >1 hour and until dry content ≥98% w/w. Yield: 2.8 kg (41%).

Mother liquor is filtered and rinsed with deionized water and EtOH (6 L each) and dried (2.2 kg). Transfer solid to reactor. Charge deionized water and EtOH (15 L each) and stir for at least 10 minutes at Ti: 20° C. Filter suspension. Charge deionized water and EtOH (6 L each) and stir for at least 5 minutes at Ta: 20° C. Filter suspension. Dry filter cake until dry content ≥90% w/w. Transfer cake into rotary flask and dry with rotovap at Ta: 60° C., p: 200-0 mbar for >1 h and until dry content ≥98% w/w. Yield: 30%. Total yield: 71% (41%+30%).

### Step 2 (can Take Place in Portions)

Amounts are L/kg related to bis-ethyl crocetinate (C20). Charge bis-ethyl crocetinate (C20) (2.5 kg) made in Step 1 is charged into the reactor. 1-Butanol (10 L/kg, 25 L), 30% NaOH (1 L/kg, 2.5 L, 3.3 kg), and EtOH (10 L/kg, 25 L) are charged into the reactor. Mixture is stirred at Ti: 50° C. for 24-72 hours. Distillate is removed at Ta: 50° C., p: 70-200 mbar (distilled volume 25 L). Purified water is added (30 L/kg, 74 L). Mixture is stirred at Ta: 50° C. for at least 12 hours. Polish filter into stir tank. Clean reactor. Return solution from stir tank. Concentrate to 11 L/kg at Ta: 50° C., p: 70-200 mbar (distillate target: 74 L). Add EtOH when needed to control foaming or as entrainer (additional EtOH is 11 L, target distillate including added EtOH is 85 L). Slowly add EtOH (30 L/kg, 74 L) at batch temperature 50° C. over a period of at least 60 minutes. Cool to Ti: 20° C. over at least 30 minutes. Transfer suspension to filter and remove mother liquor (optionally use mother liquor to rinse reactor). Charge purified water and EtOH (5 L each) and stir mixture at Ti: 20° C. for at least 1 minute. Transfer suspension to filter and remove liquor. Dry on filter for at least 5 h at p: 200-20 mbar until dry content: ≥50% w/w and transferable.

Second portion is similar to above.

Charge available products to reactor. Charge purified 1:1 water (20 L) and EtOH (15.5 kg). Stir mixture at Ti: 20° C. for at least 15 minutes. Transfer suspension to filter and remove mother liquor; use mother liquor to rinse reactor. Dry on filter for at least 5 h, p: ≤200 mbar until dry content: ≥50% w/w and transferable.

Unload material and transfer to reactor. Charge purified 1:1 water (20 L) and EtOH (20 L). Stir mixture at Ti: 20° C. for at least 15 minutes. Transfer suspension to filter and remove mother liquor; use mother liquor to rinse reactor. Dry on filter for at least 5 h, p: ≤200 mbar until dry content: ≥50% w/w and transferable.

Unload material and transfer to reactor. Charge EtOH (48 L). Stir mixture for at least 10 minutes at Ta: 20° C. Transfer suspension to filter and remove mother liquor. Charge EtOH (10 L). Stir mixture for at least 1 minute at Ta: 20° C. Transfer suspension to filter and remove liquor. Dry on filter for at least 5 h at p: 200-0 mbar until dry content: ≥98% w/w. Transfer into rotary evaporator flask and dry in the rotary evaporator for at least 5 h at Ta: 40-50° C., p: 200-0 mbar until EtOH≤0.4% w/w by NMR. Yield: 3.8 kg (81%). HPLC: 99.6% w/w. Karl-Fischer: 1.7% w/w. Na content by FAAS: 13.8%. XRPD shows product is Forms F+B.

### Example 25—Trans Sodium Crocetinate (Form H and Form H+I)

U.S. Pat. No. 8,030,350 describes a synthesis of trans sodium crocetinate in which the final step is drying the filter cake on a rotary evaporator for 5 h at 50° C. 2.186 kg TSC was obtained as an orange solid. 2.184 kg crude 1 #1 was shaken in a blender for 4 days at RT. The remaining lumps were easily ground down with a pestle. 2.183 kg crude 2 #1 (corresponded to AA-013329-Batch-01-2004) orange solid was obtained. U.S. Pat. No. 8,030,350, column 34, lines 35-49.

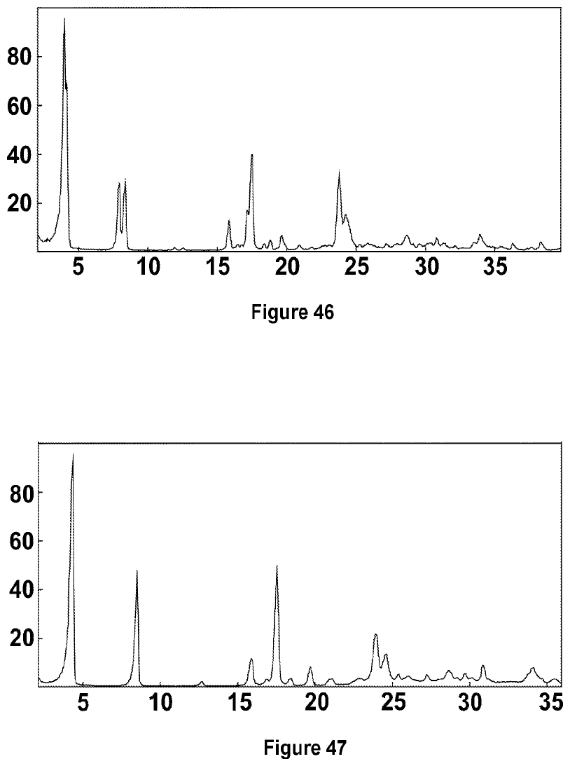

XRPDs for “AA-013329-Batch-01-2004 dried” and “AA-013329-Batch-01-2004 milled” are shown in FIG. 46 (milled) and FIG. 47 (dried). U.S. Pat. No. 8,030,350 does not show XRPDs or specify any particular form of the trans sodium crocetinate made therein. However, upon analysis of the XRPDs and using nomenclature consistent with the nomenclature used for different crystalline forms herein, FIG. 46 corresponds to a mixture of trans sodium crocetinate Form H+Form I and FIG. 47 corresponds to Form H.

XRPD measurements are made on a PANalytical powder diffractomer PW1710 (G.16.SYS.S007) using CuKα radiation. Measuring conditions: tube power 45 kV/45 mA, scan range 2°-50° (2θ), step size 0.02°, time per step 2.4 seconds. The samples are measured unground in a 0.1 mm deep sample holder.

### Example 26—Trans Sodium Crocetinate (Form E)

Form H from Example 25 is measured under different relative humidities. The humidity measurements are made on a powder diffractometer X'Pert, PANalytical (G.16.SYS.S006) using CuKα radiation. Measuring conditions: tube power 40 kV/40 mA, scan range 1°-50° (2θ), step size 0.02°, time per step 2.4 seconds. The sample is measured in a 0.4 mm deep sample holder in a closed chamber under a stream of nitrogen of variable relative humidity (RH). The humidity of the nitrogen stream is controlled by varying the relative amount of humid and dry nitrogen gas. The sample is first conditioned one hour with a dry nitrogen stream and then a first XPRD is taken. The humidity of the gas stream is then changed to 50% RH and the sample is conditioned three hours to the new RH prior to a second XRPD measurement. After 10 more hours with 50% RH, a third XRPD is taken.

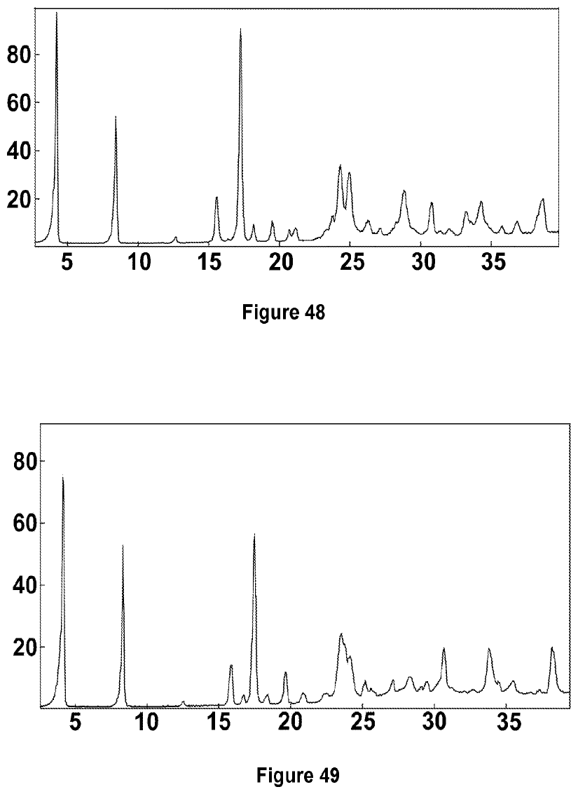

The first XRPD (Form H exposed to 0% RH) is shown in FIG. 48 and is Form G. The second XRPD (Form H exposed to 50% RH) is shown in FIG. 49 and is Form E. There is no difference between the second and third XRPDs, showing that uptake of water is complete after three hours. Exposure of Form H to different relative humidities is not discussed in U.S. Pat. No. 8,030,350.

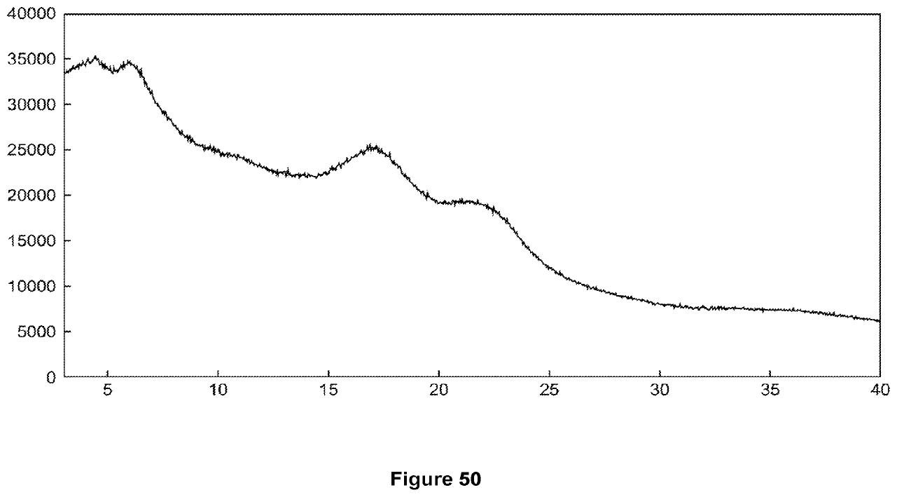

### Example 27—XRPD for FIG. 50

The Rigaku Smart-Lab X-ray diffraction system is configured for transmission Debye-Scherrer geometry using a line source X-ray beam. The x-ray source is a Cu Long Fine Focus tube operated at 40 kV and 44 mA. The Debye-Scherrer convergent beam geometry uses an X-ray mirror to focus the incident X-ray beam through the sample and onto the detector plane. This sample and incident X-ray beam are locked together during the measurement to give a constantly-illuminated sample cross section. Beam conditioning slits are used on the line X-ray source to ensure that the maximum beam width is 10 mm along the line. The inherent resolution of Debye-Scherrer geometry is governed in part by the diffractometer radius and the focusing ability of the X-ray mirror. Typically, the Rigaku Smart-Lab is operated to give peak widths of 0.1 °2θ or less. The incident slit is set at 1.00 mm, the limiting slit is set at 18.0 mm, the receiving slits are set at 20.00 mm, and the Soller slits are set at 5°. The convergent beam X-ray optics are combined with the linear D'teX detector system to give increased sensitivity to minor components and reduced measurement time. The transmission samples are prepared by loading the material between Etnom® films and the films clamped into an aluminum transmission holder to give a small environmental isolation cell. Measurements are made of the transmission profile through the isolation cell to give absolute intensity measurements for the sample. Each sample is analyzed from 3 to 40 °2θ using a continuous scan of 3 °2θ per minute with an effective step size of 0.04 °2θ. The samples are spun at 75 rpm during acquisition to improve sampling statistics.

1. A crystalline form of trans sodium crocetinate, wherein the crystalline form exhibits at least one of:
a. an XRPD pattern comprising 2-theta (°) values of 4.2, 8.4, 15.8, and 17.3, wherein the XRPD is obtained using an incident beam of Cu radiation;
b. an XRPD pattern comprising d-spacing (Å) values of 20.9, 10.5, 5.6, and 5.1.
c. an XRPD pattern comprising 2-theta (°) values of 4.1, 8.0, and 16.6, wherein the XRPD is obtained using an incident beam of Cu radiation;
d. an XRPD pattern comprising d-spacing (Å) values of 21.7, 11.0, and 5.3.
e. an XRPD pattern comprising 2-theta (°) values of 4.2, 8.4, 16.8, and 17.5, wherein the XRPD is obtained using an incident beam of Cu radiation; or
f. an XRPD pattern comprising d-spacing (Å) values of 20.9, 10.5, 5.3, and 5.1.

2. The crystalline form of trans sodium crocetinate according to claim 1, wherein the crystalline form exhibits at least one of:
a. an XRPD pattern comprising 2-theta (°) values of 4.2, 8.4, 15.8, 17.3, and 19.5, wherein the XRPD is obtained using an incident beam of Cu radiation; or
b. an XRPD pattern comprising d-spacing (Å) values of 20.9, 10.5, 5.6, 5.1, and 4.6.

3. The crystalline form of trans sodium crocetinate according to claim 2, wherein the crystalline form exhibits at least one of:
a. an XRPD pattern comprising 2-theta (°) values of 4.2, 8.4, 15.8, 16.8, 17.3, 18.4, 19.5, 21.0, 22.6, 23.9, 25.2, 25.9, 26.9, 28.6, 29.3, 29.9, 30.9, 33.5, 33.9, 35.1, and 38.3; or
b. an XRPD pattern comprising d-spacing (Å) values of 20.9, 10.5, 5.6, 5.3, 5.1, 4.8, 4.6, 4.2, 3.9, 3.7, 3.5, 3.4, 3.3, 3.1, 3.0, 2.9, 2.7, 2.6, and 2.3.

4. The crystalline form of trans sodium crocetinate according to claim 1, wherein the crystalline form exhibits at least one of:
a. an XRPD pattern comprising 2-theta (°) values of 4.1, 8.0, 16.6, 17.3, and 18.9, wherein the XRPD is obtained using an incident beam of Cu radiation; or
b. an XRPD pattern comprising d-spacing (Å) values of 21.7, 11.0, 5.3, 5.1, and 4.7.

5. The crystalline form of trans sodium crocetinate according to claim 4, wherein the crystalline form exhibits at least one of:
a. an XRPD pattern comprising 2-theta (°) values of 4.1, 8.0, 16.6, 17.3, 18.9, 23.2, 23.9, 24.1, 24.3, 25.0, 27.8, 28.0, 28.8, 31.6, 31.9, 33.6, 34.1, 35.1, and 36.4; or
b. an XPRD pattern comprising d-spacing (Å) values of 21.7, 11.0, 5.3, 5.1, 4.7, 3.8, 3.7, 3.6, 3.2, 3.1, 2.8, 2.7, 2.6, and 2.5.

6. The crystalline form of trans sodium crocetinate according to claim 1, wherein the crystalline form exhibits at least one of:
a. an XRPD pattern comprising 2-theta (°) values of 4.2, 8.4, 12.6, 16.8, 17.5, and 21.1, wherein the XRPD is obtained using an incident beam of Cu radiation; or
b. an XRPD pattern comprising d-spacing (Å) values of 20.9, 10.5, 7.0, 5.3, 5.1, and 4.2.

7. The crystalline form of trans sodium crocetinate according to claim 6, wherein the crystalline form exhibits at least one of:
a. an XRPD pattern comprising 2-theta (°) values of 4.2, 8.4, 12.6, 16.8, 17.5, 21.1, 23.9, 24.4, 25.4, 29.7, 30.8, 34.0, and 38.4, wherein the XRPD is obtained using an incident beam of Cu radiation; or
b. an XRPD pattern comprising d-spacing (Å) values of 20.9, 10.5, 7.0, 5.3, 5.1, 4.2, 3.7, 3.6, 3.5, 3.0, 2.9, 2.6, and 2.3.

8. A crystalline form of trans sodium crocetinate, wherein the crystalline form exhibits an XRPD corresponding to at least one of FIG. 1, FIG. 2, FIG. 7, FIG. 8, FIG. 12, FIG. 25, FIG. 27, FIG. 30, FIG. 32, and/or FIG. 34, wherein the XRPD is obtained using an incident beam of Cu radiation.

9. A pharmaceutical composition, wherein the pharmaceutical composition comprises a crystalline form of trans sodium crocetinate according to claim 1.

10. A process for preparing a pharmaceutical composition comprising trans sodium crocetinate, wherein the process comprises isolating and formulating a crystalline form of trans sodium crocetinate according to claim 1.

11. A pharmaceutical composition, wherein the pharmaceutical composition comprises the crystalline form of trans sodium crocetinate according to claim 2.

12. A process for preparing a pharmaceutical composition comprising trans sodium crocetinate, wherein the process comprises isolating and formulating the crystalline form of trans sodium crocetinate according to claim 2.

13. A pharmaceutical composition, wherein the pharmaceutical composition comprises the crystalline form of trans sodium crocetinate according to claim 4.

14. A process for preparing a pharmaceutical composition comprising trans sodium crocetinate, wherein the process comprises isolating and formulating the crystalline form of trans sodium crocetinate according to claim 4.

15. A pharmaceutical composition, wherein the pharmaceutical composition comprises the crystalline form of trans sodium crocetinate according to claim 6.

16. A process for preparing a pharmaceutical composition comprising trans sodium crocetinate, wherein the process comprises isolating and formulating the crystalline form of trans sodium crocetinate according to claim 6.

17. A pharmaceutical composition, wherein the pharmaceutical composition comprises the crystalline form of trans sodium crocetinate according to claim 8.

18. A process for preparing a pharmaceutical composition comprising trans sodium crocetinate, wherein the process comprises isolating and formulating the crystalline form of trans sodium crocetinate according to claim 8.
